SEQUENCE LISTING (104 sequences)

SEQ ID NO: 1 - protein, 5 aa, synthetic construct
  source 1..5: note = PD1_VH_CDR1
HYAMN

SEQ ID NO: 2 - protein, 17 aa, synthetic construct
  source 1..17: note = PD1_VH_CDR2
WINTNTGEPTYAQGFTG

SEQ ID NO: 3 - protein, 8 aa, synthetic construct
  source 1..8: note = PD1_VH_CDR3
EREPGMDN

SEQ ID NO: 4 - protein, 16 aa, synthetic construct
  source 1..16: note = PD1_VL_CDR1
RSSQSLVHANTNTYLE

SEQ ID NO: 5 - protein, 7 aa, synthetic construct
  source 1..7: note = PD1_VL_CDR2
KVSNRFS

SEQ ID NO: 6 - protein, 9 aa, synthetic construct
  source 1..9: note = PD1_VL_CDR3
FQGTHVPNT

SEQ ID NO: 7 - protein, 30 aa, synthetic construct
  source 1..30: note = PD1_HFR1
QIQLVQSGSELKKPGASVKVSCKASGYTFT

SEQ ID NO: 8 - protein, 14 aa, synthetic construct
  source 1..14: note = PD1_HFR2
WVRQAPGQGLEWMG

SEQ ID NO: 9 - protein, 32 aa, synthetic construct
  source 1..32: note = PD1_HFR3
RFVFSLDTSVSTAYLQISSLKAEDTAVYYCAR

SEQ ID NO: 10 - protein, 11 aa, synthetic construct
  source 1..11: note = PD1_HFR4
WGQGTLVTVSS

SEQ ID NO: 11 - protein, 23 aa, synthetic construct
  source 1..23: note = PD1_LFR1
DVVMTQSPLSLPVTLGQPASISC

SEQ ID NO: 12 - protein, 15 aa, synthetic construct
  source 1..15: note = PD1_LFR2
WYQQRPGQSPRLLIY

SEQ ID NO: 13 - protein, 32 aa, synthetic construct
  source 1..32: note = PD1_LFR3
GVPDRFSGSGSGTDFTLKISRVEAEDVGVYYC

SEQ ID NO: 14 - protein, 10 aa, synthetic construct
  source 1..10: note = PD1_LFR4
FGQGTKLEIK

SEQ ID NO: 15 - protein, 117 aa, synthetic construct
  source 1..117: note = PD1_VH
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 16 - protein, 112 aa, synthetic construct
  source 1..112: note = PD1_VL
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 17 - protein, 103 aa, synthetic construct
  source 1..103: note = PD1_CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 107 aa, synthetic construct [sequence omitted]

SEQ ID NO: 19 - protein, 220 aa, synthetic construct
  source 1..220: note = PD1_VH+CH1
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 219 aa, synthetic construct
  source 1..219: note = PD1_VL+CL
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 224 aa, synthetic construct
  source 1..224: note = PD1_VH+CL
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 215 aa, synthetic construct
  source 1..215: note = PD1_VL+CH1
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 327 aa, synthetic construct
  source 1..327: note = CH1+CH2+CH3 IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 24 - protein, 330 aa, synthetic construct
  source 1..330: note = CH1CH2CH3 IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 218 aa, synthetic construct
  source 1..218: note = PD1_KIH-Fc (Knob)
PAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 218 aa, synthetic construct
  source 1..218: note = PD1_KIH-Fc (Hole)
PAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 447 aa, synthetic construct
  source 1..447: note = VH_PD1-CH1-KIH-FcG1m (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 453 aa, synthetic construct
  source 1..453: note = VH_PD1-CL-KIH-FcG1m (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 447 aa, synthetic construct
  source 1..447: note = VH_PD1-CH1-KIH-FcG1m (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 5 aa, synthetic construct
  source 1..5: note = CD28_VH_CDR1
SYGVY

SEQ ID NO: 31 - protein, 16 aa, synthetic construct
  source 1..16: note = CD28_VH_CDR2
VIWAGGSTNYNSALMS

SEQ ID NO: 32 - protein, 14 aa, synthetic construct
  source 1..14: note = CD28_VH_CDR3
DHYGSYGDYYAMDY

SEQ ID NO: 33 - protein, 15 aa, synthetic construct
  source 1..15: note = CD28_VL_CDR1
RASESVEYYDTSLMQ

SEQ ID NO: 34 - protein, 7 aa, synthetic construct
  source 1..7: note = CD28_VL_CDR2
AASSVES

SEQ ID NO: 35 - protein, 9 aa, synthetic construct
  source 1..9: note = CD28_VL_CDR3
QQSRKVPWT

SEQ ID NO: 36 - protein, 30 aa, synthetic construct
  source 1..30: note = CD28_HFR1
QVKLQESGPGLVAPSQSLSITCTVSGFSLT

SEQ ID NO: 37 - protein, 14 aa, synthetic construct
  source 1..14: note = CD28_HFR2
WVRQPPGKGLEWLG

SEQ ID NO: 38 - protein, 32 aa, synthetic construct
  source 1..32: note = CD28_HFR3
RLSISKDNSKSQVFLKMNSLQTDDTAMYYCAR

SEQ ID NO: 39 - protein, 11 aa, synthetic construct
  source 1..11: note = CD28_HFR4
WGQGTTVTVSS

SEQ ID NO: 40 - protein, 23 aa, synthetic construct
  source 1..23: note = CD28_LFR1
DIVLTQSPASLAVSLGQRATISC

SEQ ID NO: 41 - protein, 15 aa, synthetic construct
  source 1..15: note = CD28_LFR2
WYQQKPGQPPKLLIY

SEQ ID NO: 42 - protein, 32 aa, synthetic construct
  source 1..32: note = CD28_LFR3
GVPARFSGSGSGTDFSLNIHPVEEDDIAMYFC

SEQ ID NO: 43 - protein, 10 aa, synthetic construct
  source 1..10: note = CD28_LFR4
FGGGTKLEIK

SEQ ID NO: 44 - protein, 122 aa, synthetic construct
  source 1..122: note = CD28_VH
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSS

SEQ ID NO: 45 - protein, 111 aa, synthetic construct
  source 1..111: note = CD28_VL
DIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIK

SEQ ID NO: 46 - protein, 105 aa, synthetic construct
  source 1..105: note = CD28_CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 47 - protein, 107 aa, synthetic construct [sequence omitted]

SEQ ID NO: 48 - protein, 227 aa, synthetic construct
  source 1..227: note = CD28_VH+CH1
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 49 - protein, 219 aa, synthetic construct
  source 1..219: note = CD28_VL-CL
DIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 229 aa, synthetic construct
  source 1..229: note = CD28_VH-CL
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 216 aa, synthetic construct
  source 1..216: note = CD28_VL-CH1
DIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 52 - protein, 451 aa, synthetic construct
  source 1..451: note = VH_CD28-CH1-FcG4m
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 53 - protein, 455 aa, synthetic construct
  source 1..455: note = VH_CD28-CL-KIH-FcG1m (Hole)
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 218 aa, synthetic construct; identical to SEQ ID NO: 26
  source 1..218: note = CD28_KIH-Fc (Hole)

SEQ ID NO: 55 - protein, 478 aa, synthetic construct
  source 1..478: note = scFv-VH-VL-CD28-KIH-FcG1m (Hole)
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKLLEDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 479 aa, synthetic construct
  source 1..479: note = scFv-VL-VH-CD28-KIH-FcG1m (Hole)
DIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKGGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSRTLEDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 15 aa, synthetic construct
  source 1..15: note = linker
GGGGSGGGGSGGGGS

SEQ ID NO: 58 - protein, 9 aa, synthetic construct
  source 1..9: note = hinge
DKTHTCPPC

SEQ ID NO: 59 - protein, 478 aa, synthetic construct
  source 1..478: note = scFvCD28VHVL-VL-CH1-PD1
QVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKGGGGSGGGGSGGGGSDVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - protein, 478 aa, synthetic construct
  source 1..478: note = VLCH1PD1-scFvCD28VHVL
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIK

SEQ ID NO: 61 - protein, 710 aa, synthetic construct
  source 1..710: note = VHPD1-CH1-KIH-FcG1m-(G4S)3-scFvCD28VHVL (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIK

SEQ ID NO: 62 - protein, 709 aa, synthetic construct
  source 1..709: note = VHPD1-CH1-KIH-FcG1m-(G4S)3-scFvCD28VLVH (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSS

SEQ ID NO: 63 - protein, 478 aa, synthetic construct
  source 1..478: note = scFvCD28VLVH-VLCH1PD1
DIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKGGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSSGGGGSGGGGSGGGGSDVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 478 aa, synthetic construct
  source 1..478: note = VLCH1_PD1-scFvCD28_VLVH
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSDIVLTQSPASLAVSLGQRATISCRASESVEYYDTSLMQWYQQKPGQPPKLLIYAASSVESGVPARFSGSGSGTDFSLNIHPVEEDDIAMYFCQQSRKVPWTFGGGTKLEIKGGGGSGGGGSGGGGSQVKLQESGPGLVAPSQSLSITCTVSGFSLTSYGVYWVRQPPGKGLEWLGVIWAGGSTNYNSALMSRLSISKDNSKSQVFLKMNSLQTDDTAMYYCARDHYGSYGDYYAMDYWGQGTTVTVSS

SEQ ID NO: 65 - protein, 5 aa, synthetic construct
NSGMH

SEQ ID NO: 66 - protein, 17 aa, synthetic construct
VIWYDGSKRYYADSVKG

SEQ ID NO: 67 - protein, 4 aa, synthetic construct
NDDY

SEQ ID NO: 68 - protein, 11 aa, synthetic construct
RASQSVSSYLA

SEQ ID NO: 69 - protein, 7 aa, synthetic construct
DASNRAT

SEQ ID NO: 70 - protein, 9 aa, synthetic construct
QQSSNWPRT

SEQ ID NO: 71 - protein, 113 aa, synthetic construct [sequence omitted]

SEQ ID NO: 72 - protein, 107 aa, synthetic construct [sequence omitted]

SEQ ID NO: 73 - protein, 5 aa, synthetic construct
NYYMY

SEQ ID NO: 74 - protein, 16 aa, synthetic construct
GINPSNGGTNFNEKFK

SEQ ID NO: 75 - protein, 11 aa, synthetic construct
RDYRFDMGFDY

SEQ ID NO: 76 - protein, 15 aa, synthetic construct
RASKGVSTSGYSYLH

SEQ ID NO: 77 - protein, 7 aa, synthetic construct
LASYLES

SEQ ID NO: 78 - protein, 9 aa, synthetic construct
QHSRDLPLT

SEQ ID NO: 79 - protein, 120 aa, synthetic construct [sequence omitted]

SEQ ID NO: 80 - protein, 111 aa, synthetic construct [sequence omitted]

SEQ ID NO: 81 - protein, 246 aa, synthetic construct
  source 1..246: note = scFv antiCD28 clone 2 VL-G4S3-VH-FcG1B-KIH
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKKLGGGGSGGGGSGGGGSRTQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 82 - protein, 245 aa, synthetic construct
  source 1..245: note = scFv antiCD28 clone 3 VL-G4S3-VH-FcG1B-KIH
DIQMTQSPSSLSASVGDRVTITCHASQNIYVYLNWYQQKPGKAPKLLIYKASNLHTGVPSRFSGSGSGTDFTLTISSLQPEDFATYYCQQGQTYPYTFGGGTKVEIKLGGGGSGGGGSGGGGSRTQVQLVQSGAEVKKPGASVKVSCKASGYTFTSYYIHWVRQAPGQGLEWIGSIYPGNVQTNYNEKFKDRATLTVDTSISTAYMELSRLRSDDTAVYFCTRSHYGLDFNFDVWGQGTTVTVSS

SEQ ID NO: 83 - protein, 122 aa, synthetic construct
  source 1..122: note = Clone 1 mutVH_FRs1
QVTLKESGPGLVKPTETLTITCTVSGFSLTSYGVYWVRQPPGKALEWLGVIWAGGSTNYNSALMSRLTISKDNSKSQVFLTMTSMDPVDTAMYYCARDHYGSYGDYYAMDYWGQGTTVTVSS

SEQ ID NO: 84 - protein, 122 aa, synthetic construct
  source 1..122: note = Clone 1 mutVH_FRs2
QVQLVESGGGVVQPGRSLRLSCAVSGFSLTSYGVYWVRQAPGKGLEWLGVIWAGGSTNYNSALMSRLTISKDNSKSTVFLQMTSLRAEDTAMYYCARDHYGSYGDYYAMDYWGQGTLVTVSS

SEQ ID NO: 85 - protein, 122 aa, synthetic construct
  source 1..122: note = Clone 1 mutVH_FRs3
QVQLQESGPGLVKPSETLSITCTVSGFSLTSYGVYWVRQPPGKGLEWLGVIWAGGSTNYNSALMSRLTISKDNSKSQVFLKMSSVTAADTAMYYCARDHYGSYGDYYAMDYWGQGTLVTVSS

SEQ ID NO: 86 - protein, 111 aa, synthetic construct
  source 1..111: note = Clone 1 mutVL_FRs1
DIQLTQSPSSLSASVGDRVTISCRASESVEYYDTSLMQWYQQKPGKAPKLLIYAASSVESGVPSRFSGSGSGTDFSLTISSLQPEDIAMYFCQQSRKVPWTFGGGTKVEIK

SEQ ID NO: 87 - protein, 111 aa, synthetic construct
  source 1..111: note = Clone 1 mutVL_FRs2
DIVLTQSPDSLAVSLGERATISCRASESVEYYDTSLMQWYQQKPGQPPKLLIYAASSVESGVPDRFSGSGSGTDFSLTISSVQAEDVAMYFCQQSRKVPWTFGGGTKVEIK

SEQ ID NO: 88 - protein, 122 aa, synthetic construct
  source 1..122: note = Clone 1 mutVH_CDRs1
QVKLQESGPGLVAPSQSLSITCTVSGFSLSSYGVYWVRQPPGKGLEWLGVIWAGGSTNYNSALMSRLSISKDNSKSQVFLKMNSLQTDDTAMYYCARDHYGSYGDYYAMDYWGQGTTVTVSS

SEQ ID NO: 89 - protein, 111 aa, synthetic construct
  source 1..111: note = Clone 1 mutVL_CDRs1
DIVLTQSPASLAVSLGQRATISCRASQSVEYYDTSLMQWYQQKPGQPPKLLIYAASSVESGVPARFSGSGSGTDFSLNIHPVEEDDIAMYFCQQSRKVPWTFGGGTKLEIK

SEQ ID NO: 90 - protein, 111 aa, synthetic construct
  source 1..111: note = Clone 1 mutVL_CDRs2
DIVLTQSPASLAVSLGQRATISCRASESVEYYDTSLMQWYQQKPGQPPKLLIYAASSRESGVPARFSGSGSGTDFSLNIHPVEEDDIAMYFCQQSRKVPWTFGGGTKLEIK

SEQ ID NO: 91 - protein, 8 aa, synthetic construct
GFSLTSYG

SEQ ID NO: 92 - protein, 7 aa, synthetic construct
IWAGGST

SEQ ID NO: 93 - protein, 16 aa, synthetic construct
ARDHYGSYGDYYAMDY

SEQ ID NO: 94 - protein, 10 aa, synthetic construct
ESVEYYDTSL

SEQ ID NO: 95 - skipped

SEQ ID NO: 96 - protein, 9 aa, synthetic construct
QQSRKVPWT

SEQ ID NO: 97 - protein, 25 aa, synthetic construct
QVKLQESGPGLVAPSQSLSITCTVS

SEQ ID NO: 98 - protein, 17 aa, synthetic construct
VYWVRQPPGKGLEWLGV

SEQ ID NO: 99 - protein, 38 aa, synthetic construct
NYNSALMSRLSISKDNSKSQVFLKMNSLQTDDTAMYYC

SEQ ID NO: 100 - protein, 11 aa, synthetic construct
WGQGTTVTVSS

SEQ ID NO: 101 - protein, 26 aa, synthetic construct
DIVLTQSPASLAVSLGQRATISCRAS

SEQ ID NO: 102 - protein, 17 aa, synthetic construct
MQWYQQKPGQPPKLLIY

SEQ ID NO: 103 - protein, 37 aa, synthetic construct
SSVESGVPARFSGSGSGTDFSLNIHPVEEDDIAMYFC

SEQ ID NO: 104 - protein, 10 aa, synthetic construct
FGGGTKLEIK

(19) United States
(12) Patent Application Publication
(10) Pub. No.: US 2026/0049141 A1
(43) Pub. Date: Feb. 19, 2026

(54) MULTIFUNCTIONAL MOLECULE DIRECTED AGAINST CD28

(71) Applicant: OSE IMMUNOTHERAPEUTICS, NANTES (FR)

(72) Inventors: NICOLAS POIRIER, GRANDCHAMPS DES FONTAINES (FR); CAROLINE MARY, SAINTE-PAZANNE (FR); AURORE MORELLO, SAINT SEBASTIEN SUR LOIRE (FR); MARION COLONELLO, REZE (FR)

(21) Appl. No.: 19/100,208

(22) PCT Filed: Aug. 2, 2023
(86) PCT No.: PCT/EP2023/071400

§ 371 Date: Jan. 31, 2025

(30) Foreign Application Priority Data
Aug. 2, 2022 (EP) 22306169.8

(51) Int. Cl. C07K 16/28; A61K 38/00; A61P 35/00; C07K 16/46

(52) U.S. Cl. CPC C07K 16/2818; A61K 38/00; A61P 35/00; C07K 16/468; C07K 2317/31; C07K 2317/55; C07K 2317/565; C07K 2317/622; C07K 2317/75

(57) ABSTRACT

The present invention provides a multifunctional molecule comprising an agonistic anti-CD28 binding domain covalently linked to another binding moiety that binds to a target specifically expressed on T cells surface, and uses thereof.

### FIELD OF THE INVENTION

The invention pertains to the field of medicine, especially immunotherapy. The present invention provides a multifunctional molecule comprising a single anti-CD28 binding domain covalently linked to a binding moiety that binds to a target specifically expressed on immune cells surface.

### BACKGROUND OF THE INVENTION

The ability of T-cells to recognize and kill their cellular targets depends on a coordinated set of interactions. The prime example of these instructions is the recognition and binding of the target cell by the T-cell Receptor (TCR) complex. T-cell activation can be further promoted by additional interactions, such as CD28. T-cells present CD28 on their surface, which can provide a co-stimulatory signal to augment the activation via the TCR complex. When a T-cell recognizes its target cell via its TCR complex, and then also engages the co-stimulatory signal via CD28 binding to its the target cell, T-cell activation is enhanced.

Agonistic anti-CD28 mAbs can be applied in sustained ex vivo expansion of cultured T-cells. However, the use of antibodies against CD28 has been discouraged as a result of a series of acute and serious adverse events in a phase I clinical trial where super agonist anti-CD28 mAb was tested systemically (HGnig, Nature Reviews Immunology. 2012; 12:317-318). Localized or targeted use of anti-CD28 mAb can be used with less risk for promotion of antitumor immunity (Jung et al., Int J Cancer. 2001 Jan. 15; 91 (2):225-30).

Thus, there is a need to develop improved agents for safe immunotherapy targeting human CD28, notably for the treatment of cancer.

An axis of therapy investigated in the prior art is to reduce non-specific targeting, more specifically by preparing bispecific antibodies directed against both CD28 and a tumor specific antigen such as PD-1. It allows to achieve direct targeting of the tumor in combination with the costimulatory effect of CD28 and blocking the tumor targeted checkpoint inhibition effect of PD-1/PD-L1. Examples of such antibodies can be found in WO2022/094299 and WO2022/081886.

### SUMMARY OF THE INVENTION

The inventors provided for the first time a multifunctional molecule which is capable of binding to CD28 on one side and to another target specifically expressed on human T cells on the other side, especially PD-1. Surprisingly, this multifunctional molecule does not cause the expected problem despite the combination of an agonist of CD28 and of another T cell specific antigen binding domain, even if this combination would be considered as deleterious at first sight given the results of preceding trials with anti-CD28 super agonist.

Furthermore, this multifunctional molecule exhibits a cis-targeting and a cis-activating ability. Indeed, it binds through the two binding domains to a single T cell, thereby causing an efficient but controlled stimulation of T cells, without risking any form of overstimulation.

Additionally, despite its lack of tumor-specific binding ability, it is able to target T cells located in or in close vicinity of the tumor, again avoiding a systemic activation of T cells without any specificity for tumors.

The molecules of the present invention are particularly suitable for the prevention and/or treatments of several diseases, in particular of cancers.

Accordingly, the inventors provide a multifunctional molecule comprising a single anti-CD28 binding domain covalently linked to a binding moiety that binds to a target specifically expressed on T cells surface for numerous therapeutic applications, in particular for the treatment of cancer.

The multifunctional molecule of the invention is capable of cis-binding to a T cell. This cis-targeting ability specifically activates T cells which express both CD28 and another T specific antigen, such as PD-1, and does not activate naïve non-specific T cells, thus avoiding non-specific activation and limiting the toxicity of the molecule of the invention.

Additionally, the multifunctional molecule of the invention does not exhibit trans-activity, i.e., co-targeting tumor cells and T cells, circumventing potential unwanted side effects.

The multifunctional molecule of the invention therefore fulfills the goal of providing a multifunctional molecule efficient in the treatment of several conditions, including cancer, but with few or no side effect.

In a first aspect, the invention concerns a multifunctional molecule comprising or consisting essentially of:
(a) a first binding moiety, said first binding moiety binding to a target specifically expressed on T cells surface selected from the group consisting of PD-1, VISTA, CTLA-4, BTLA, TIGIT, CD160, LAG3 and TIM3; and
(b) a second binding moiety, said second binding moiety having an agonistic effect on CD28 and being an anti-CD28 antigen binding domain comprising a heavy chain variable domain (VH) and a light chain variable domain (VL) and optionally a heavy chain constant domain (CH1) and a light chain constant domain (CL), wherein:
(i) the heavy chain variable domain (VH) comprises complementary determining regions (CDRs) HCDR1, HCDR2 and HCDR3, and
(ii) the light chain variable domain (VL) comprises complementary determining regions (CDRs) LCDR1, LCDR2 and LCDR3,
   wherein:
the heavy chain CDR1 (HCDR1) comprises or consists of an amino acid sequence of SEQ ID NO: 30, optionally with one, two or three modification(s) selected from substitution(s), addition(s), deletion(s) and any combination thereof;
the heavy chain CDR2 (HCDR2) comprises or consists of an amino acid sequence of SEQ ID NO: 31, optionally with one, two or three modification(s) selected from substitution(s), addition(s), deletion(s) and any combination thereof;
the heavy chain CDR3 (HCDR3) comprises or consists of an amino acid sequence of SEQ ID NO: 32;
optionally with one, two or three modification(s) selected from substitution(s), addition(s), deletion(s) and any combination thereof;
the light chain CDR1 (LCDR1) comprises or consists of an amino acid sequence of SEQ ID NO: 33, optionally with one, two or three modification(s) selected from substitution(s), addition(s), deletion(s) and any combination thereof;
the light chain CDR2 (LCDR2) comprises or consists of an amino acid sequence of SEQ ID NO: 34, optionally with one, two or three modification(s) selected from substitution(s), addition(s), deletion(s) and any combination thereof; and
the light chain CDR3 (LCDR3) comprises or consists of an amino acid sequence of SEQ ID NO: 35, optionally with one, two or three modification(s) selected from substitution(s), addition(s), deletion(s) and any combination thereof;
   wherein said multifunctional molecule comprises a single anti-CD28 antigen binding domain.

Preferably, the target specifically expressed on T cells surface is PD-1.

In some embodiments, the first binding moiety is a first antigen binding domain comprising a VH and a VL and optionally a light chain constant domain (CL) and a heavy chain constant domain (CH1), and wherein said first antigen binding domain is linked to the N-terminal end of a first Fc chain and the molecule further comprises a second Fc chain forming with the first Fc chain a Fc domain.

Preferably, the anti-CD28 antigen binding comprises a VH and a VL and optionally a CL and a CH1, preferably comprising:
(i) a heavy chain variable domain (VH) comprising HCDR1, HCDR2 and HCDR3, and
(ii) a light chain variable domain (VL) comprising LCDR1, LCDR2 and LCDR3,
   wherein:
the heavy chain CDR1 (HCDR1) comprises or consists of an amino acid sequence of SEQ ID NO: 30;
the heavy chain CDR2 (HCDR2) comprises or consists of an amino acid sequence of SEQ ID NO: 31;
the heavy chain CDR3 (HCDR3) comprises or consists of an amino acid sequence of SEQ ID NO: 32;
the light chain CDR1 (LCDR1) comprises or consists of an amino acid sequence of SEQ ID NO: 33;
the light chain CDR2 (LCDR2) comprises or consists of an amino acid sequence of SEQ ID NO: 34,
the light chain CDR3 (LCDR3) comprises or consists of an amino acid sequence of SEQ ID NO:35;
   even more preferably comprising (a) a VH comprising or consisting of an amino acid sequence of SEQ ID NO: 44, optionally with one, two or three modification(s) selected from substitution(s), addition(s), deletion(s) and any combination thereof; and (b) a VL comprising or consisting of an amino acid sequence of SEQ ID NO: 45, optionally with one, two or three modification(s) selected from substitution(s), addition(s), deletion(s) and any combination thereof.

Particularly, the anti-CD28 antigen binding domain is covalently linked to: (i) the C-terminal end of a CL of the first binding moiety, (ii) the C-terminal end of a CH1 of the first binding moiety, (iii) the N-terminal end of a VH of the first binding moiety, or (iii) the N-terminal end of a VL of the first binding moiety, optionally through a peptide linker.

In some embodiments, the molecule comprises a first Fc chain and a second Fc chain forming together a Fc domain and the anti-CD28 antigen binding domain is covalently linked to the C-terminal end of one of the Fc chains, preferably of the first Fc chain covalently linked to at its N-terminal end to the first antigen binding domain, optionally through a peptide linker.

In some embodiments, the molecule comprises a first Fc chain and a second Fc chain forming together a Fc domain, the anti-CD28 antigen binding domain is covalently linked by its C-terminal end to N-terminal end of the second Fc chain and the first antigen binding domain is covalently linked by its C-terminal end to N-terminal end of the first Fc chain, optionally through a peptide linker.

Preferably, the anti-CD28 antigen binding domain is a Fab, a CrossMAb or a scFv, preferably a scFv.

In particular, in the multifunctional according to the invention:
the anti-CD28 antigen binding domain is a scFv with, from the N-terminal end to the C-terminal end, a VH linked to a VL, preferably by a peptide linker; or,
the anti-CD28 antigen binding domain is a scFv with, from the N-terminal end to the C-terminal end, a VL linked to a VH, preferably by a peptide linker.

In some embodiments, the anti-CD28 antigen binding domain is a Fab or a CrossMAb.

Particularly, in the anti-CD28 antigen binding domain (i) the heavy chain variable domain (VH) further comprises heavy chain variable region framework regions (HFR) HFR1, HFR2, HFR3 and HFR4 comprising or consisting of an amino acid sequence of SEQ ID Nos: 36, 37, 38 and 39, respectively, optionally with one, two or three modification(s) selected from substitution(s), addition(s), deletion(s) and any combination thereof, and
(ii) the light chain variable domain (VL) further comprises region framework regions (LFR) LFR1, LFR2, LFR3 and LFR4, comprising or consisting of an amino acid sequence of SEQ ID NOs: 40, 41, 42 and 43, respectively, optionally with one, two or three modification(s) selected from substitution(s), addition(s), deletion(s) and any combination thereof.

More particularly, the anti-CD28 antigen binding domain comprises: (a) a heavy chain variable region (VH) comprising or consisting of an amino acid sequence selected from the group consisting of SEQ ID NO: 44, 83, 84, 85 and 88, optionally with one, two or three amino acid modification(s) selected from substitution(s), addition(s), deletion(s) and any combination thereof, preferably substitution(s); and (b) a light chain variable region (VL) comprising or consisting of an amino acid sequence selected from the group consisting of SEQ ID NO: 45, 86, 87, 89 and 90, optionally with one, two or three amino acid modification(s) selected from substitution(s), addition(s), deletion(s) and any combination thereof, preferably substitution(s).

Preferably, the anti-CD28 antigen binding domain comprises: (a) a VH comprising or consisting of an amino acid sequence of SEQ ID NO: 44, optionally with one, two or three modification(s) selected from substitution(s), addition(s), deletion(s) and any combination thereof, preferably in the framework regions; and (b) a VL comprising or consisting of an amino acid sequence of SEQ ID NO: 45, optionally with one, two or three modification(s) selected from substitution(s), addition(s), deletion(s) and any combination thereof, preferably in the framework regions.

In some embodiment, in the multifunctional molecule of the invention, the first binding moiety is or is from an anti-PD-1 antibody selected from the group consisting of Pembrolizumab, Nivolumab, Pidilizumab, Cemiplimab, Camrelizumab, AUNP12, AMP-224, AGEN-2034, Tisleizumab, PDR001, MK-3477, PF-06801591, JNJ-63723283, genolimzumab, LZM-009, BCD-100, SHR-1201, BAT-1306, AK-103, MEDI-0680, JS001, BI-754091, CBT-501, INCSHR1210, TSR-042, GLS-010, AM-0001, STI-1110, MGA012, or IBI308, preferably Pembrolizumab or Nivolumab.

In some embodiments, the first binding moiety is an anti-PD-1 antigen binding domain, preferably comprising:
(i) a VH comprising HCDR1, HCDR2 and HCDR3, and (ii) a VL comprising LCDR1, LCDR2 and LCDR3, wherein:
   the heavy chain CDR1 (HCDR1) comprises or consists of an amino acid sequence of SEQ ID NO: 1;
   the heavy chain CDR2 (HCDR2) comprises or consists of an amino acid sequence of SEQ ID NO: 2;
   the heavy chain CDR3 (HCDR3) comprises or consists of an amino acid sequence of SEQ ID NO: 3;
   the light chain CDR1 (LCDR1) comprises or consists of an amino acid sequence of SEQ ID NO: 4;
   the light chain CDR2 (LCDR2) comprises or consists of an amino acid sequence of SEQ ID NO: 5, and
   the light chain CDR3 (LCDR3) comprises or consists of an amino acid sequence of SEQ ID NO:6,
      preferably wherein said the anti-PD-1 antigen binding domain is a F(ab′)2, a Fab or a CrossMAb.

Preferably, the first binding moiety is an anti-PD-1 antigen binding domain, preferably comprising: (a) a VH comprising or consisting of an amino acid sequence of SEQ ID NO: 15, optionally with one, two or three modification(s) selected from substitution(s), addition(s), deletion(s) and any combination thereof; preferably in the framework regions; and (b) a VL comprising or consisting of an amino acid sequence of SEQ ID NO: 16, optionally with one, two or three modification(s) selected from substitution(s), addition(s), deletion(s) and any combination thereof, preferably in the framework regions.

Preferably, the anti-PD-1 antigen binding domain is a F(ab′)2, a Fab or a CrossMAb.

In some embodiments, the first binding moiety is an anti-PD-1 antigen binding domain comprising:
a) (i) a VH comprising HCDR1, HCDR2 and HCDR3, and (ii) a VL comprising LCDR1, LCDR2 and LCDR3, wherein: the heavy chain CDR1 (HCDR1) comprises or consists of an amino acid sequence of SEQ ID NO: 65; the heavy chain CDR2 (HCDR2) comprises or consists of an amino acid sequence of SEQ ID NO: 66; the heavy chain CDR3 (HCDR3) comprises or consists of an amino acid sequence of SEQ ID NO: 67; the light chain CDR1 (LCDR1) comprises or consists of an amino acid sequence of SEQ ID NO: 68; the light chain CDR2 (LCDR2) comprises or consists of an amino acid sequence of SEQ ID NO: 69, and the light chain CDR3 (LCDR3) comprises or consists of an amino acid sequence of SEQ ID NO: 70; or
b) i) a VH comprising or consisting of an amino acid sequence of SEQ ID NO: 71; and ii) a VL comprising or consisting of an amino acid sequence of SEQ ID NO: 72; or
c) (i) a VH comprising HCDR1, HCDR2 and HCDR3, and (ii) a VL comprising LCDR1, LCDR2 and LCDR3, wherein: the heavy chain CDR1 (HCDR1) comprises or consists of an amino acid sequence of SEQ ID NO: 73; the heavy chain CDR2 (HCDR2) comprises or consists of an amino acid sequence of SEQ ID NO: 74; the heavy chain CDR3 (HCDR3) comprises or consists of an amino acid sequence of SEQ ID NO: 75; the light chain CDR1 (LCDR1) comprises or consists of an amino acid sequence of SEQ ID NO: 76; the light chain CDR2 (LCDR2) comprises or consists of an amino acid sequence of SEQ ID NO: 77, and the light chain CDR3 (LCDR3) comprises or consists of an amino acid sequence of SEQ ID NO: 78; or
d) i) a VH comprising or consisting of an amino acid sequence of SEQ ID NO: 79; and ii) a VL comprising or consisting of an amino acid sequence of SEQ ID NO: 80.

In a second aspect, the invention concerns an isolated nucleic acid sequence or a group of isolated nucleic acid sequences encoding the multifunctional molecule according to the invention, or a vector comprising said isolated nucleic acid sequence or group of isolated nucleic acid sequences.

In a third aspect, the invention concerns a host cell, comprising the vector or the isolated nucleic acid or group of isolated nucleic acid molecules of the invention.

In a fourth aspect, the invention relates to a method for producing the multifunctional molecule according to the invention, comprising a step of culturing a host cell according to the invention and a step of isolating the multifunctional molecule.

In a fifth aspect, the invention concerns a pharmaceutical composition comprising the multifunctional molecule according to the invention, the nucleic acid or group of nucleic acid molecules or vector according to the invention or the host cell according to the invention and a pharmaceutically acceptable carrier.

In particular, the pharmaceutical composition, multifunctional molecule, nucleic acid, group of nucleic acid molecules, vector, host cell according to the invention are for use as a medicament.

Particularly, the pharmaceutical composition, multifunctional molecule, nucleic acid, group of nucleic acid molecules, vector, host cell according to the invention, are for use in the treatment of cancer or in the treatment of an infectious disease.

Preferably, the disease is a cancer selected from the group consisting of brain cancer, carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers.

Alternatively, the disease is an infectious disease caused by a virus selected from the group consisting of Human immunodeficiency virus, hepatitis virus, herpes virus, adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, Human T-lymphotropic virus, dengue virus, papillomavirus, molluscum virus, poliovirus, rabies virus, JC virus and arboviral encephalitis virus.

In some embodiments, the pharmaceutical composition, multifunctional molecule, nucleic acid, group of nucleic acid molecules, vector, host cell according to the invention, for use in combination with radiotherapy or with an additional therapeutic agent, in particular a chemotherapeutic agent or an anti-infectious agent.

Particularly, the additional therapeutic agent is selected from the group consisting of chemotherapeutic agent, anti-infectious agent, alkylating agents, angiogenesis inhibitors, antibodies, antimetabolites, antimitotic, antiproliferative, antivirals, aurora kinase inhibitors, apoptosis promoters, activators of death receptor pathway, Bcr-Abl kinase inhibitors, Bi-Specific T cell Engager antibody drug conjugates, Bruton's tyrosine kinase (BTK) inhibitors, cyclin-dependent kinase inhibitors, cell cycle inhibitors, cyclooxygenase-2 inhibitors, leukemia viral oncogene homolog receptor inhibitors, growth factor inhibitors, heat shock protein-90 inhibitors, histone deacetylase inhibitors, hormonal therapies, inhibitors of apoptosis proteins, antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs, poly ADP-ribose polymerase inhibitors, platinum chemotherapeutics, polo-like kinase inhibitors, phosphoinositide-3 kinase inhibitors, proteasome inhibitors, purine analogs, pyrimidine analogs, receptor tyrosine kinase inhibitors, retinoids, small inhibitory ribonucleic acids (siRNAs), topoisomerase inhibitors, ubiquitin ligase inhibitors, hypomethylating agents, checkpoints inhibitors, peptide vaccine, cancer vaccine, epitopes or neoepitopes from tumor antigens, as well as combinations of one or more of these agents.

Finally, the invention concerns a method for treating a disease such as a cancer or an infectious disease in a subject in need thereof, comprising the administration of a multifunctional molecule or pharmaceutical composition of the invention, preferably in an effective amount.

The invention also concerns the use of a multifunctional molecule or pharmaceutical composition according to the invention for the manufacture of a medicament, for the treatment of a disease such as a cancer or an infectious disease.

### DETAILED DESCRIPTION OF THE INVENTION

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” such as Fab, Fab′, F(ab′)2, Fv, single chain (scFv), CrossMAb, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody. In terms of structure, an antibody may have heavy (H) chains and light (L) chains interconnected by disulfide bonds. There are two types of light chain, lambda (λ) and kappa (κ). Each heavy and light chain contains a constant region and a variable region (or “domain”). Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, and U.S. Department of Health and Human Services, 1991, which is hereby incorporated by reference). Preferably, the CDRs are defined according to Kabat or IGMT method, preferably by the Kabat method. The framework regions act to form a scaffold that provides, for positioning the CDRs in correct orientation by inter-chain, non-covalent interactions. The CDRs are primarily responsible for binding to an epitope of an antigen. The CDRs of each chain are typically referred to as “Complementarity Determining Region 1” or “CDR1”, “CDR2”, and “CDR3”, numbered sequentially starting from the N-terminus. The VL and VH domain of the antibody according to the invention may comprise four framework regions or “FR's”, which are referred to in the art and herein as “Framework region 1” or “FR1”, “FR2”, “FR3”, and “FR4”, respectively. These framework regions and complementary determining regions are preferably operably linked in the following order: FR1-CDR1-FR2-CDR2-FR3-CDR3-FR4 (from amino terminus to carboxy terminus).

The “CrossMab technology” is based on a domain crossover between heavy and light chains thereby creating different domain arrangements for heavy chains and light chains of different specificity. WO 2009/080251, WO 2009/080252, WO 2009/080253, WO 2009/080254 and Schaefer, W. et al, PNAS, 108 (201 1) 11187-1191 (the disclosure thereof being incorporated herin by reference) relate to bivalent, bispecific IgG antibodies with a domain crossover. In CrossMab, (i) the constant domains CL and CH1 are replaced by each other, and/or (ii) the constant domains VL and VH are replaced by each other. Accordingly, the antigen binding domain comprises a chain with VH and CL and another chain with VL and CH1. As used herein, the term “CrossMAb” refers to antigen binding domains with an inversion of CL and CH1 domains, in particular in one binding arm of antibodies. Thus, such binding domain comprises a VH domain linked to a CL domain and a VL domain linked to a CH1 domain. Such format reduces the byproduct formation caused by a mismatch of a light chain of a first binding domain that specifically binds to a first antigen with the wrong heavy chain of a second binding domain that specifically binds to a second antigen (when compared to approaches without such CL-CH1 domain exchanges). CrossMAb typically has a Fab conformation but with the above CL and CH1 inversion. They are for example described in WO 2009/080253 and Schaefer, W. et al, PNAS, 108 (2011) 11187-1191, the disclosure of which being incorporated herein by reference.

An “antibody heavy chain” as used herein, refers to the larger of the two types of polypeptide chains present in antibody conformations. An heavy chain is comprised of a heavy chain variable region (abbreviated herein as VH) and a heavy chain constant region. The heavy chain constant region is comprised of three domains, CH1, CH2 and CH3. The VH region can be further subdivided into regions of hypervariability, termed complementarity determining regions (CDR), interspersed with regions that are more conserved, termed framework regions (FR). The VH is particularly composed of three CDRs and four FRs, arranged from amino-terminus to carboxy-terminus in the following order: FR1, CDR1, FR2, CDR2, FR3, CDR3, FR4. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; κ and λ light chains refer to the two major antibody light chain isotypes. A light chain is comprised of a light chain variable region (abbreviated herein as VL) and a light chain constant region (CL). The light chain constant region is comprised of one domain, CL. The VL region can be further subdivided into regions of hypervariability, termed complementarity determining regions (CDR), interspersed with regions that are more conserved, termed framework regions (FR). A VL is particularly composed of three CDRs and four FRs, arranged from amino-terminus to carboxy-terminus in the following order: FR1, CDR1, FR2, CDR2, FR3, CDR3, FR4. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

As used herein, an “antigen-binding fragment” or “antigen-binding domain” of an antibody means a part of an antibody, i.e. a molecule corresponding to a portion of the structure of the antibody of the invention, that exhibits antigen-binding capacity for a particular antigen, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof. Such antigen-binding domain could be for instance Fab, scFv or CrossMab.

As used herein, the term “humanized antibody” is intended to refer to antibodies in which CDR sequences derived from the germline of another mammalian species, such as a mouse, have been grafted onto human framework sequences (e.g., chimeric antibodies that contain minimal sequence derived from a non-human antibody). A “humanized form” of an antibody, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. Additional framework region modifications may be made within the human framework sequences. Preferably humanized antibody has a T20 humanness score greater than 80%, 85% or 90%. “Humanness” of an antibody can for example be measured using the T20 score analyzer to quantify the humanness of the variable region of antibodies as described in Gao S H, Huang K, Tu H, Adler A S. BMC Biotechnology. 2013: 13:55 or via a web-based tool to calculate the T20 score of antibody sequences using the T20 Cutoff Human Databases: http://abAnalyzer.lakepharma.com.

By “chimeric antibody” is meant an antibody made by combining genetic material from a nonhuman source, preferably such as a mouse, with genetic material from a human being. Such antibody derives from both human and non-human antibodies linked by a chimeric region. Chimeric antibodies generally comprise constant domains from human and variable domains from another mammalian species, reducing the risk of a reaction to foreign antibodies from a non-human animal when they are used in therapeutic treatments.

As used herein, the terms “fragment crystallizable region” “Fc region” or “Fc domain” are interchangeable and refers to the tail region of an antibody that interacts with cell surface receptors called Fc receptors. The Fc region or domain is typically composed of two domains, optionally identical, derived from the second and third constant domains of the antibody's two heavy chains (i.e., CH2 and CH3 domains). Portion of the Fc domain refers to the CH2 or the CH3 domain. Optionally, the Fc region or domain may optionally comprise all or a portion of the hinge region between CH1 and CH2. Accordingly, the Fc domain may comprise the hinge, the CH2 domain and the CH3 domain. Optionally, the Fc domain is that from IgG1, IgG2, IgG3 or IgG4, optionally with IgG1 hinge-CH2-CH3 and IgG4 hinge-CH2-CH3.

With regard to the “binding” capacity of the binding moieties described herein, the terms “bind” or “binding” refer to antibodies including antibody fragments and derivatives that recognize and contact another peptide, polypeptide, protein or molecule. The terms “specific binding”, “specifically binds to,” “specific for,” “selectively binds” and “selective for” a particular target mean that the antigen binding domain recognizes and binds a specific target, but does not substantially recognize or bind other molecules in a sample. For example, an antibody that specifically (or preferentially) binds to an antigen is an antibody that binds the antigen for example with greater affinity, avidity, more readily, and/or with greater duration than it binds to other molecules. Preferably, the term “specific binding” means the contact between an antibody (or antigen binding domain) and an antigen with a binding affinity equal or lower than 10⁻⁷ M. In certain aspects, antibodies bind with affinities equal or lower than 10⁻⁸ M, 10⁻⁹ M or 10⁻¹⁰ M.

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e., starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn formation | A, C, D, E, G, H, K, N, Q, R, S, P, and T |
| Flexible residues | E, Q, T, K, S, G, P, D, E, and R |

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. Said alignment of sequences can be carried out by well-known methods in the art, for example, using the algorithm for global alignment of Needleman-Wunsch. Protein analysis software matches similar sequences using measures of similarity assigned to various substitutions, deletions, and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on:

www.ebi.ac.uk/Tools/services/web/toolform.ebi?tool=emboss_needle&context=protein, for example using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

Alternatively, Sequence identity can also be typically determined using sequence analysis software Clustal Omega using the HHalign algorithm and its default settings as its core alignment engine. The algorithm is described in Söding, J. (2005) ‘Protein homology detection by HMM-HMM comparison’. Bioinformatics 21, 951-960, with the default settings.

“Eu numbering” (also known as Eu index) refers to the antibody numbering system (Kabat et al., 1991, Sequences of Proteins of Immunological Interest, 5th Ed., United States Public Health Service, National Institutes of Health, Bethesda), which is based on the sequential numbering of the first human IgG1 sequenced (the Eu antibody; Edelman, et al., 1969, Proc Natl Acad Sci USA 63: 78-85).

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds.

As used herein, a “pharmaceutical composition” refers to a preparation of one or more of the active agents, such as comprising a multifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one aspect, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g., the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain aspects, such a term refers to the improvement or eradication of a disease, a disorder, an infection or symptoms associated with it. In other aspects, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g., an illness that disrupts normal physical or mental functions. More preferably, the term disorder refers to immune and/or inflammatory diseases that affect animals and/or humans, such as cancer.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4+ T cells, CD8+ T cells, T helper 1 type T cells, T helper 2 type T cells, T helper 17 type T cells and inhibitory T cells.

As used herein, the term “T effector cell”, “T eff” or “effector cell” describes a group of immune cells that includes several T cell types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, cytotoxic T cells and helper T cells (Th1 and Th2).

As used herein, the term “regulatory T cell”, Treg cells” or “T reg” refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

The term “exhausted T cell” refers to a population of T cell in a state of dysfunction (i.e., “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “effector memory stem like T cell” refers to a subset of tumor-reactive intra-tumoral T cells bearing hallmarks of exhausted cells and central memory cells, including expression of the checkpoint protein PD-1 and the transcription factor Tcf1. These cells can be called Tcf1⁺PD-1⁺CD8⁺ T cells. These cells reside in the tumor microenvironment and are critical for immune control of cancer promoted by immunotherapy. They are critical for maintaining the T cell response during chronic viral infection and cancer, and provide the proliferative burst seen after PD-1 immunotherapy. These cells undergo a slow self-renewal and also give rise to the more terminally differentiated exhausted CD8 T cells. These cells and their characteristics are further defined in the following articles, the disclosure thereof being incorporated herein by reference: Siddiqui et al, 2019, Immunity, 50, 195-211; and Jadhav et al, 2019, PNAS, 116, 14113-14118).

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues.

The term “antagonist” as used herein, refers to a molecule that blocks or reduces the biological activity or functionality of another molecule. Particularly, this term refers to an antibody or a fragment thereof that binds to a cellular receptor (e.g., PD-1) as a reference substance (e.g., PD-L1 and/or PD-L2), preventing it from producing all or part of its usual biological effects (e.g., the creation of an immune suppressive microenvironment). The antagonist activity of a humanized antibody according to the invention may be assessed by competitive ELISA.

As used herein the term “agonist” refers to a molecule that induces and/or increases the biological activity, function and/or expression of another molecule. According to specific embodiments, the agonist induces and/or increases the co-stimulatory effect on an immune cell (e.g. T cells). According to specific embodiments, the agonist induces and/or increases signaling to an immune cell (e.g. T cell).

Particularly, this term refers to an antibody or fragment thereof that binds to CD28 and stimulates/induces co-stimulatory signals required for T cell action. The agonist activity of a humanized antibody according to the invention may be assessed by measurement of IL-2 secretion in T cell bioassay.

As used herein, the terms “pharmacokinetics” and “PK” are used interchangeably and refer to the fate of compounds, substances or drugs administered to a living organism. Pharmacokinetics particularly comprise the ADME or LADME scheme, which stands for Liberation (i.e., the release of a substance from a composition), Absorption (i.e., the entrance of the substance in blood circulation), Distribution (i.e., dispersion or dissemination of the substance through the body) Metabolism (i.e., transformation or degradation of the substance) and Excretion (i.e., the removal or clearance of the substance from the organism). The two phases of metabolism and excretion can also be grouped together under the title elimination. Different pharmacokinetics parameters can be monitored by the man skilled in the art, such as elimination half-life, elimination constant rate, clearance (i.e., the volume of plasma cleared of the drug per unit time), Cmax (Maximum serum concentration), and Drug exposure (determined by Area under the curve, see Scheff et al, Pharm Res. 2011 May; 28(5):1081-9) among others.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1.5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e., “about 1, 2 and 3” refers to about 1, about 2 and about 3). Further, when a listing of values is described herein (e.g., about 50%, 60%, 70%, 80%, 85% or 86%) the listing includes all intermediate and fractional values thereof (e.g., 54%, 85.4%).

The term “essentially” as used herein in connection with any given biological sequence means said biological sequence varies from the reference sequence contained in the sequence listing by up to 10% of the biological sequence length. In particular, by “consists essentially of” is intended that the biological sequence consists of that sequence, but it may also include 1, 2, 3, 4, 5, 6, 7, 8, 9 or 10 substitutions, additions, deletions or a mixture thereof, preferably 1, 2, 3, 4, or 5 substitutions, additions, deletions or a mixture thereof, with the proviso that said biological sequence varies from the reference sequence contained in the sequence listing by up to 10% of the biological sequence length.

### Multifunctional Molecule Structure

In a first aspect, the invention concerns a multifunctional molecule comprising:
(a) a first binding moiety, said first binding moiety binding to a target specifically expressed on T cells; and
(a) a second binding moiety, said second binding moiety being an agonistic anti-CD28 antigen binding domain;
said multifunctional molecule comprising a single anti-CD28 antigen binding domain.

The first and second binding moieties are directed to two different targets, so that the molecule according to the invention is “multifunctional”, i.e., is able to bind to at least two different targets (e.g., CD28 and a target specifically expressed on T cells surface such as PD-1). The first and second binding moieties are directed to two different targets, so that the molecule according to the invention is particularly “bifunctional”, i.e., is able to bind to two different targets (e.g., CD28 and a target specifically expressed on T cells surface such as PD-1).

The multifunctional molecule may particularly comprise two, three or four binding moieties, but only one binding moiety against CD28. For example, the multifunctional molecule may comprise:
one binding moiety that binds to a target specifically expressed on T cells surface and one binding moiety that binds to CD28; or
two binding moieties that bind to a target specifically expressed on T cells surface, said two binding moieties binding to the same or to different target(s); and one binding moiety that binds to CD28; or
three binding moieties that bind to a target specifically expressed on T cells surface, said three binding moieties binding to the same or to different target(s); and one binding moiety that binds to CD28.

The first and second binding moiety are preferably antigen binding domains, in particular derived from Fab, Fab′, F(ab′)2, Fv, single chain (scFv), CrossMAb or nanobody (VHH), preferably a F(ab′)2, a Fab, a CrossMAb or a scFV. In the multifunctional molecule, the first and second binding moiety may have the same or different formats. For example, the first and second binding moiety can be both Fab or scFV. Alternatively, the first and second binding moieties are not both scFv. In a preferred aspect, the anti-CD28 antigen binding moiety is a scFv and the binding moiety binding to a target specifically expressed on T cells surface is not an scFv. For example, the first binding moiety may be a F(ab′)2, a CrossMAb or a Fab, and the second binding moiety may be a scFv, a Fab or a CrossMAb.

In particular, the first binding moiety is a F(ab′)2, a CrossMAb or a Fab, and the second binding moiety is a scFv.

The multifunctional molecule preferably comprises: i) a first and a second binding moiety, i) optionally a CH1, ii) optionally a hinge, iii) optionally a CH2 domain and a CH3 domain.

In the context of IgG antibodies, the IgG isotypes each have three heavy chain constant regions (CH). Accordingly, “CH” domains in the context of IgG are as follows: “CH1” refers to the first constant domain (most N-terminal) of three constant domains of the heavy chain. It particularly refers to positions 118-215 according to the EU index as in Kabat. “Hinge” refers to positions 216-230 according to the EU index as in Kabat. “CH2” refers to the second constant domain and in particular to positions 231-340 according to the EU index as in Kabat, and “CH3” refers to the third constant domain (most C-terminal) and in particular to positions 341-447 according to the EU index as in Kabat. The CH domains may be naturally occurring CH domains, or naturally occurring CH domains in which one or more amino acids have been substituted.

The term “hinge region” refers to the flexible polypeptide comprising the amino acids between the CH1 and CH2 domains of an antibody. The hinge is defined structurally for the purposes of the present invention. The IgG1 “hinge region” as used herein comprises residues 216-230 according to the according to the EU index as in Kabat.

The first and/or second binding moiety may comprise a CL domain.

In the context of IgG antibodies, the IgG isotypes each have a light chain constant domain (CL). “CL domain” refers to the light chain immunoglobulin constant domain that is located C-terminally to the VL domain. It spans about EU index Kabat positions 107-216. A CL domain may be a naturally occurring CL domain, or a naturally occurring CL domain in which one or more amino acids have been substituted.

Such multifunctional molecule preferably comprises a first Fc chain and a second Fc chain that are complementary and that together form a Fc domain. The first and second binding moieties can be on the same or different Fc chain, thereby forming a homodimer Fc domain or a heterodimer Fc domain, respectively.

Then, the multifunctional molecule may thus comprise two binding moieties, preferably two antigen binding domains and a Fc domain. Preferably, the multifunctional molecule of the invention comprises a first and a second antigen binding domains and a Fc domain. In addition, the multifunctional molecule may also comprise a hinge domain.

In addition, the multifunctional molecule may also comprise one or several peptide linkers, that allow linkage between different part of the multifunctional molecule (e.g., between a Fc chain and a binding moiety).

In an embodiment, the first and second binding moieties are Fab, so that the multifunctional molecule preferably comprises or consists of:
(i) a first light chain comprising a variable domain (VL) and a constant domain (CL),
(ii) a first heavy chain comprising a variable domain (VH) and a constant domain (CH1), said first light and heavy chains forming the first binding moiety that binds to a target specifically expressed on T cells surface;
(iii) a second light chain comprising a variable domain (VL) and a constant domain (CL),
(iv) a second heavy chain comprising a variable domain (VH) and a constant domain (CH1), said second light and heavy chains forming the second binding moiety that binds to CD28.

Such a molecule may further comprise a Fc domain so that the multifunctional molecule preferably comprises two different Fab domains and a Fc domain. In particular, the multifunctional comprises or consists of:
(i) a first light chain comprising a variable domain (VL) and a constant domain (CL),
(ii) a first heavy chain comprising a variable domain (VH), a first constant domain (CH1), optionally a hinge, a second constant domain (CH2) and a third constant domain (CH3), said CH2 and CH3 domains forming the first Fc chain,
wherein said first light and heavy chains form the first binding moiety that binds to a target specifically expressed on T cells surface, and
(iii) a second light chain comprising a variable domain (VL) and a constant domain (CL),
(iv) a second heavy chain comprising a variable domain (VH) a first constant domain (CH1), optionally a hinge, a second constant domain (CH2) and a third constant domain (CH3), said CH2 and CH3 domains forming the second Fc chain,
wherein said second light and heavy chains form the second binding moiety that binds to CD28, and
   wherein the first and second Fc chains are complementary and form a Fc domain.

In an embodiment, the first binding moiety is a Fab and the second binding moiety is a CrossMAb, so that the multifunctional molecule preferably comprises or consists of:
(i) a first chain that is a light chain comprising a variable domain (VL) and a constant domain (CL),
(ii) a second chain that is a heavy chain comprising a variable domain (VH) and a constant domain (CH1),
said light and heavy chains forming the first binding moiety that binds to a target specifically expressed on T cells surface;
(iii) a third chain comprising a variable domain (VH) and a constant domain (CL),
(iv) a fourth chain comprising a variable domain (VL) and a constant domain (CH1),
   said first and second chains forming the second binding moiety that binds to CD28.

Such molecule may further comprise a Fc domain so that the multifunctional molecule preferably comprises two different Fab domains and a Fc domain. In particular, the multifunctional comprises or consists of:
(i) a first chain that is light chain comprising a variable domain (VL) and a constant domain (CL),
(ii) a second chain that is a heavy chain comprising a variable domain (VH), a first constant domain (CH1), optionally a hinge, a second constant domain (CH2) and a third constant domain (CH3); said CH2 and CH3 domains forming the first Fc chain,
wherein said first and second chains form the first binding moiety that binds to a target specifically expressed on immune cells surface,
(iii) a third chain comprising a variable domain (VH), a first constant domain (CL), optionally a hinge, a second constant domain (CH2) and a third constant domain (CH3); said CH2 and CH3 domains forming the second Fc chain; and
(iv) a fourth chain comprising a variable domain (VL) and a constant domain (CH1),
wherein said third and fourth chains form the second binding moiety that binds to CD28, and
   wherein the first and second Fc chains are complementary and form a Fc domain.

In another embodiment, the first binding moiety is a Fab and the second binding moiety is a scFv so that the multifunctional molecule preferably comprises or consists of:
(i) a first light chain comprising a variable domain (VL) and a constant domain (CL),
(ii) a first heavy chain comprising a variable domain (VH) and a constant domain (CH1), wherein said first light and heavy chains form the first binding moiety that binds to a target specifically expressed on T cells surface and form a Fab,
(iii) a second light chain comprising a variable domain (VL), and
(iv) a second heavy chain comprising a variable domain (VH),
wherein said second light and heavy chains are linked by a peptide linker and form a scFv that binds to CD28.

Such molecule may further comprise a Fc domain so that the multifunctional molecule comprises a Fab, a scFv and a Fc domain. In particular, the multifunctional comprises or consists of:
(i) a first light chain comprising a variable domain (VL) and a constant domain (CL),
(ii) a first heavy chain comprising a variable domain (VH), a first constant domain (CH1), optionally a hinge, a second constant domain (CH2) and a third constant domain (CH3), said CH2 and CH3 domains forming the first Fc chain,
wherein said first light and heavy chains form the first binding moiety that binds to a target specifically expressed on T cells surface and form a Fab,
(iii) a second light chain comprising a variable domain (VL),
(iv) a second heavy chain comprising a variable domain (VH),
wherein said second light and heavy chain are linked by a peptide linker and form a scFv that binds to CD28, and
(v) a second Fc chain, complementary to the first Fc chain to form a Fc domain.

In particular, the multifunctional molecule of the invention comprises or consists of:
(i) a first chain comprising or consisting essentially of a light chain variable domain (VL) and a light chain constant domain (CL),
(ii) a second chain comprising or consisting essentially of a heavy chain variable domain (VH), a first heavy chain constant domain (CH1), optionally a hinge, a second heavy chain constant domain (CH2) and a third heavy chain constant domain (CH3), said CH2 and CH3 domains forming a first Fc chain,
wherein said first and second chains form the first binding moiety that binds to a target specifically expressed on T cells surface and form a Fab, such as PD-1;
(iii) a third chain comprising or consisting essentially of a) a variable domain (VL) linked to a variable domain (VH), preferably by a peptide linker, forming a second binding moiety that is a scFV that binds to CD28, and b) optionally a hinge and a second Fc chain, complementary to the first Fc chain to form a Fc domain.
wherein third chain form a second binding moiety that binds to CD28.

In an embodiment, the first binding moiety is a CrossMAb and the second binding moiety is a ScFV, so that the multifunctional molecule preferably comprises or consists of:
(i) a first chain comprising a light chain variable domain (VL) and a heavy chain constant domain (CH1),
(ii) a second chain comprising a heavy chain variable domain (VH) and a light chain constant domain (CL),
said first and second chains forming the CrossMAb that binds to a target specifically expressed on T cells surface, preferably to PD-1;
(iii) a third chain comprising a variable domain (VH) and a variable domain (VL), preferably linked by a peptide linker, said third chain forming a scFv that binds to CD28.

Such molecule may further comprise a Fc domain. In particular, the multifunctional molecule comprises or consists of:
(i) a first chain comprising a heavy chain variable domain (VH) and a light chain constant domain (CL),
(ii) a second chain comprising a light chain variable domain (VL) and a heavy chain constant domain (CH1), a second constant domain (CH2) and a third constant domain (CH3); said CH2 and CH3 domains forming the first Fc chain,
wherein the first and second chains form a first binding moiety that binds to a target specifically expressed on immune cells surface, such as PD-1;
(iii) a third chain comprising a) a variable domain (VL) linked to a variable domain (VH), preferably by a peptide linker, forming a second binding moiety that is a scFV that binds to CD28, and b) a second Fc chain, complementary to the first Fc chain to form a Fc domain.

In particular, the scFv may be linked either at the N-terminal end of the second Fc chain, at the C-terminal end of the first or second Fc chain, or at the C-terminal end of the CL of the first binding moiety, preferably at the N-terminal end of the second Fc chain or the C-terminal end of the first Fc chain, even more preferably at the N-terminal end of the second Fc chain.

In a particular embodiment, the second Fc chain is devoid of any binding moiety, immunotherapeutic agent or other protein.

Alternatively, the second Fc chain can be linked at its C-terminal end to another binding moiety or immunotherapeutic agent or protein, that is not a moiety binding to CD28.

In another embodiment, the first binding moiety is a F(ab′)2 and the second binding moiety is a scFv.

In particular, that the multifunctional molecule comprises or consists of:
(i) a first light chain comprising a variable domain (VL) and a constant domain (CL),
(ii) a first heavy chain comprising a variable domain (VH) and a constant domain (CH1),
(iii) a second light chain comprising a variable domain (VL) and a constant domain (CL), and
(iv) a second heavy chain comprising a variable domain (VH) and a constant domain (CH1),
wherein said first and second light chains and said second light and heavy chains form the first binding moiety F(ab′)2 that binds to a target specifically expressed on T cells surface,
(v) a third light chain comprising a variable domain (VL),
(vi) a third heavy chain comprising a variable domain (VH),
wherein said third light and heavy chains are linked by a peptide linker and form the second binding moiety (scFv) that binds to CD28.

In a particular embodiment, the F(ab′)2 comprises a Fab and a CrossMab, so that the multifunctional molecule comprises or consists of:
(i) a first light chain comprising a variable domain (VL) and a constant domain (CH1),
(ii) a first heavy chain comprising a variable domain (VH) and a constant domain (CL),
(iii) a second light chain comprising a variable domain (VL) and a constant domain (CL),
(iv) a second heavy chain comprising a variable domain (VH) and a constant domain (CH1),
wherein said first and second light chains and said first and second heavy chains form the first binding moiety that binds to a target specifically expressed on T cells surface,
(v) a third light chain comprising a variable domain (VL),
(vi) a third heavy chain comprising a variable domain (VH),
wherein said third light and heavy chains are linked by a peptide linker and form the second binding moiety (scFv) that binds to CD28.

In particular, the multifunctional molecule comprises or consists of:
(i) a first chain comprising or consisting essentially of a light chain variable domain (VL) and a light chain constant domain (CL),
(ii) a second chain comprising or consisting essentially of a heavy chain variable domain (VH) and a heavy chain constant domain (CH1),
(iii) a third chain comprising or consisting essentially of a light chain variable domain (VL) and a light chain constant domain (CL),
(iv) a fourth chain comprising or consisting essentially of a heavy chain variable domain (VH) and a heavy chain constant domain (CH1),
wherein said first, second, third and fourth chains form a first F(ab′)2 binding moiety that binds to a target specifically expressed on T cells surface, such as PD-1; and
(v) a fifth chain comprising or consisting essentially of a variable domain (VL) linked to a variable domain (VH), preferably by a peptide linker, forming a second binding moiety that is a scFV that binds to CD28.

Such molecule may further comprise a Fc domain so that the multifunctional molecule comprises a F(ab′)2, a scFv and a Fc domain. In particular, the multifunctional comprises or consists of:
(i) a first light chain comprising a variable domain (VL) and a constant domain (CL),
(ii) a first heavy chain comprising a variable domain (VH) and a constant domain (CH1) optionally a hinge, a second constant domain (CH2) and a third constant domain (CH3); said CH2 and CH3 domains forming the first Fc chain,
(iii) a second light chain comprising a variable domain (VL) and a constant domain (CL),
(iv) a second heavy chain comprising a variable domain (VH) and a constant domain (CH1), optionally a hinge, a second constant domain (CH2) and a third constant domain (CH3); said CH2 and CH3 domains forming the second Fc chain,
wherein the first and second Fc chains are complementary and form a Fc domain, and wherein said first and second light chains and said first and second heavy chains form the first binding moiety (antibody) that binds to a target specifically expressed on T cells surface,
(v) a third light chain comprising a variable domain (VL),
(vi) a third heavy chain comprising a variable domain (VH),
wherein said third light and heavy chains are linked by a peptide linker and form the second binding moiety (scFv) that binds to CD28.

In particular, the multifunctional comprises or consists of:
(i) a first chain comprising or consisting essentially of a light chain variable domain (VL) and a light chain constant domain (CL),
(ii) a second chain comprising or consisting essentially of a heavy chain variable domain (VH) and a heavy chain constant domain (CH1) optionally a hinge, a second heavy chain constant domain (CH2) and a third heavy chain constant domain (CH3); said CH2 and CH3 domains forming a first Fc chain,
(iii) a third chain comprising or consisting essentially of a light chain variable domain (VL) and a light chain constant domain (CL),
(iv) a fourth chain comprising or consisting essentially of a heavy chain variable domain (VH) and a first heavy chain constant domain (CH1), optionally a hinge, a second heavy chain constant domain (CH2) and a third heavy chain constant domain (CH3); said CH2 and CH3 domains forming a second Fc chain,
wherein the first and second Fc chains are complementary and form a Fc domain, and wherein said first, second, third, fourth chains form a first binding moiety that is an antibody that binds to a target specifically expressed on T cells surface, such as PD-1; and
(vi) a fifth chain comprising or consisting essentially of a variable domain (VL) linked to a variable domain (VH), preferably by a peptide linker, forming a second binding moiety that is a scFV that binds to CD28.

In an embodiment, the molecule comprise a F(ab′)2 comprising a Fab and a CrossMAb, a scFv and a Fc domain. In particular, the multifunctional comprises or consists of:
(i) a first light chain comprising a variable domain (VL) and a constant domain (CH1),
(ii) a first heavy chain comprising a variable domain (VH) and a constant domain (CL), optionally a hinge, a second constant domain (CH2) and a third constant domain (CH3); said CH2 and CH3 domains forming the first Fc chain
(iii) a second light chain comprising a variable domain (VL) and a constant domain (CL),
(iv) a second heavy chain comprising a variable domain (VH) and a constant domain (CH1), optionally a hinge, a second constant domain (CH2) and a third constant domain (CH3) said CH2 and CH3 domains forming the first Fc chain;
wherein said first and second light chains and said first and second heavy chains form the first binding moiety (antibody) that binds to a target specifically expressed on T cells surface
(v) a third light chain comprising a variable domain (VL),
(vi) a third heavy chain comprising a variable domain (VH),
wherein said third light and heavy chains are linked by a peptide linker and form the second binding moiety (scFv) that binds to CD28.

In particular, the scFv may be linked either at the C-terminal end of the first or second Fc chain, at the C-terminal end of the CL of the first or second light chain of the first binding moiety or at the N-terminal end of the VH or VL of the first or second light chain of the first binding moiety.

In an embodiment, the first and second binding moieties are both scFv, so that the multifunctional molecule preferably comprises or consists of:
(i) a first light chain comprising a variable domain (VL), and
(ii) a first heavy chain comprising a variable domain (VH),
said first light and heavy chains being linked by a peptide linker and forming the first binding moiety that binds to a target specifically expressed on T cells surface;
(iii) a second light chain comprising a variable domain (VL),
(iv) a second heavy chain comprising a variable domain (VH),
said second light and heavy chains being linked by a peptide linker and forming the second binding moiety that binds to CD28.

Such molecule may further comprise a Fc domain so that the multifunctional molecule comprises or consists of:
(i) a first light chain comprising a variable domain (VL),
(ii) a first heavy chain comprising a variable domain (VH), optionally a hinge, CH2 and CH3 domains; said CH2 and CH3 domains forming the first Fc chain,
wherein said first light and heavy chains are linked by a peptide linker and form the first binding moiety that binds to a target specifically expressed on T cells surface; and,
(iii) a second light chain comprising a variable domain (VL) and a constant domain (CL),
(iv) a second heavy chain comprising a variable domain (VH) a first constant domain (CH1), optionally a hinge, a second constant domain (CH2) and a third constant domain (CH3); said CH2 and CH3 domains forming the second Fc chain,
wherein said second light and heavy chains are linked by a peptide linker and form the second binding moiety that binds to CD28 and,
wherein the first and second Fc chains are complementary to form a Fc domain.

The different elements composing the multifunctional molecule of the invention are more particularly described below.

### Binding Moieties

According to the invention, the multifunctional molecule comprises:
(a) a first binding moiety, said first binding moiety binding to a target specifically expressed on T cells surface, in particular T cells; and
(a) a second binding moiety, said second binding moiety being an agonist of CD28 and being an anti-CD28 antigen binding domain;
said multifunctional molecule comprising a single anti-CD28 antigen binding domain.

As mentioned above, the multifunctional molecule may particularly comprise two, three or four binding moieties binding to a target specifically expressed on T cells surface, but only one binding moiety binding CD28. For example, multifunctional molecule may comprise one, two or three binding moiety that binds to a target specifically expressed on T cells surface and one binding moiety that binds to CD28.

The binding moieties are more particularly described here below. In the section “First binding moieties” are examples of binding moieties that can serve as the “one, two or three binding moiety that binds to a target specifically expressed on T cells surface” that may be comprised in the multifunctional molecule according to the invention. In the section “Second binding moieties” are examples of binding moieties against CD28.

The first and second binding moiety are preferably antigen binding domains, in particular derived from Fab, Fab′, F(ab′)2, Fv, single chain (scFv), CrossMAb or nanobody (VHH), preferably a F(ab′)2, a Fab, a CrossMAb or a scFV. In the multifunctional molecule, the first and second binding moiety may have the same or different formats. In a particular aspect, the first and second binding moieties are not both scFv. In a preferred aspect, the anti-CD28 antigen binding moiety is a scFv and the binding moiety binding to a target specifically expressed on immune cells surface, in particular T cells is not an scFv. In one embodiment, the first or second binding moiety is a CrossMab.

Preferably, the anti-CD28 antigen binding moiety is a scFv and the binding moiety binding to a target specifically expressed on immune cells surface comprises or consists of a F(ab′)2, a Fab or a CrossMAb.

### First Binding Moiety

The multifunctional molecule according to the invention comprises a first binding moiety, said first binding moiety binding to a target specifically expressed on T cells, in particular T cells that are present in a tumoral environment (e.g., that are recruited on tumoral site), for example PD-1 positive T cells.

The target specifically expressed on T cells surface is selected from the group consisting of PD-1, CTLA-4, BTLA, TIGIT, CD160, CD40L, ICOS, CD27, OX40, 4-1BB, GITR, HVEM, Tim-1, LFA-1, TIM3, CD39, CD30, NKG2D, NKG2A, LAG3, 2B4, DR3, CD101, CD44, CD38, CXCR3, CXCR5, CD4, CD8, CD25, CRTAM, CD96, CD226, CD112R, CD103, CEACAM and CD122. Preferably, the target specifically expressed on T cells surface is selected from the group consisting of PD-1, VISTA, CTLA-4, BTLA, TIGIT, CD160, LAG3 and TIM3, and preferably is PD-1. These markers are, in particular, not antigens of the TCR pathway (interaction between antigen presenting cells and T cells).

Preferably, the target specifically expressed on T cells surface is a target that allows the interaction between a T cell and a tumor cell (for example: PD-1 on T cells and PD-L1 on tumoral cells). In particular, the target specifically expressed on T cells surface is not a target that allows the interaction between a T cell and an antigen presenting cell (APC) via the TCR pathway.

As used herein, the term “target” of the binding moiety refers to a carbohydrate, lipid, peptide, polypeptide, protein, antigen or epitope that is specifically recognized or targeted by the first binding moiety according to the invention and expressed on the external surface of T cells. With regards to the expression of a target on the surface of T cells, the term “expressed” refers to a target, such as carbohydrates, lipids, peptides, polypeptides, proteins, antigens or epitopes that are present or presented at the outer surface of a cell.

In one aspect, the target is specifically expressed by T cells in a healthy subject or in a subject suffering from a disease, in particular such as a cancer. In one aspect, the target is specifically expressed by T cells in a subject suffering from a disease such as a cancer. Preferably, the target has a higher expression level in T cells in a subject suffering from a disease such as a cancer than in an healthy subject. This means that the target has a higher expression level in T cells than in other cells or that the ratio of T cells expressing the target by the total immune cells is higher than the ratio of other cells expressing the target by the total other cells. Preferably the expression level or ratio is higher by a factor 2, 5, 10, 20, 50 or 100. More specifically, it can be determined for T cells, more specifically CD4+ T cells, CD8+ T cells, effector T cells or exhausted T cells, or in a particular context, for instance a subject suffering of a disease such as a cancer or an infection.

“T cell” or “T lymphocytes” as used herein includes for example CD4+ T cells, CD8+ T cells, T helper 1 type T cells, T helper 2 type T cells, T regulator, T helper 17 type T cells and inhibitory T cells. In a very particular aspect, the T cell is an exhausted T cell or an effector memory stem like T cell.

The target can be a receptor expressed at the surface of T cells. The receptor can be an inhibitor receptor. Alternatively, the receptor can be an activating receptor.

Advantageously, the first binding moiety does not bind to CD28 or to any variant or mutant thereof.

Alternatively or additionally, the first binding moiety does not bind to CD28 ligand(s), i.e., B7.1 and B7.2 or to any variant or mutant thereof.

Such targets expressed on T cells are more particularly described in the Table D below.

TABLE D
Examples of target of interest.

| Name | Official name | UniProt reference |
|---|---|---|
| 2B4 | Natural killer cell receptor 2B4 (NK cell type I receptor protein 2B4, | Q07763 |
| | NKR2B4) (Non-MHC restricted killing associated) (SLAM family | |
| | member 4, SLAMF4) (Signaling lymphocytic activation molecule 4) | |
| | (CD antigen CD244) | |
| 4-1BB | Tumor necrosis factor receptor superfamily member 9 (4-1BB ligand | Q07011 |
| | receptor, CD137) | |
| BTLA | B- and T-lymphocyte attenuator (B- and T-lymphocyte-associated | Q7Z6A9 |
| | protein) (CD antigen CD272) | |
| CD101 | Immunoglobulin superfamily member 2, IgSF2 (Cell surface | Q93033 |
| | glycoprotein V7) (Glu-Trp-Ile EWI motif-containing protein 101, EWI- | |
| | 101) (CD antigen CD101) | |
| CD160 | CD160 antigen (Natural killer cell receptor BY55) | 095971 |
| CD25 | IL-2 receptor subunit alpha; IL-2-RA; IL-2R subunit alpha; IL2-RA | P01589 |
| CD96 | T-cell surface protein tactile, Cell surface antigen CD96, T | P40200 |
| | cell-activated increased late expression protein | |
| CD4 | T-cell surface glycoprotein CD4 or T-cell surface antigen T4/Leu-3 | P01730 |
| CD8a | T-cell surface glycoprotein CD8 alpha chain or T-lymphocyte | P01732 |
| | differentiation antigen T8/Leu-2 | |
| CD8b | T-cell surface glycoprotein CD8 beta chain | P10966 |
| CD30 | Tumor necrosis factor ligand superfamily member 8 (CD30 ligand, | P32971 |
| | CD30-L) (CD antigen CD153) | |
| CD38 | ADP-ribosyl cyclase/cyclic ADP-ribose hydrolase 1 (ADPRC 1, cADPr | P28907 |
| | hydrolase 1) | |
| CD39 | Ectonucleoside triphosphate diphosphohydrolase-1 (NTPDase 1, | P49961 |
| | Ecto-apyrase, ATPDase 1, or Lymphoid cell activation antigen) | |
| CD40L | CD40 ligand (T-cell antigen Gp39, TNF-related activation protein, | P29965 |
| | Tumor necrosis factor ligand superfamily member 5, CD154) | |
| CD44 | CD44 antigen (Epican, Extracellular matrix receptor III, GP90 | P16070 |
| | lymphocyte homing/adhesion receptor, HUTCH-I, Heparan sulfate | |
| | proteoglycan, Hermes antigen, Hyaluronate receptor, Phagocytic | |
| | glycoprotein 1, Phagocytic glycoprotein I) | |
| CTLA-4 | Cytotoxic T-lymphocyte protein 4 (Cytotoxic T-lymphocyte-associated | P16410 |
| | antigen 4, CTLA-4) (CD antigen CD152) | |
| DR3 | Death receptor 3 (Tumor necrosis factor receptor superfamily | Q93038 |
| | member 25, WSL, Apo-3, LARD) | |
| GITR | Tumor necrosis factor receptor superfamily member 18 (Activation- | Q9Y5U5 |
| | inducible TNFR family receptor, Glucocorticoid-induced TNFR-related | |
| | protein, CD357) | |
| HVEM | Tumor necrosis factor receptor superfamily member 14 (Herpes virus | Q92956 |
| | entry mediator A, Herpesvirus entry mediator A, HveA) (Tumor | |
| | necrosis factor receptor-like 2, TR2) (CD antigen CD270) | |
| ICOS | Inducible T-cell costimulator (Activation-inducible lymphocyte | Q9Y6W8 |
| | immunomediatory molecule, CD278) | |
| LAG3 | Lymphocyte activation gene 3 protein, LAG-3 (Protein FDC) (CD | P18627 |
| | antigen CD223) | |
| LFA-1 | Leukocyte adhesion glycoprotein LFA-1 alpha chain (Integrin alpha-L, | P20701 |
| | CD11 antigen-like family member A) | |
| NKG2D | NKG2-D type Il integral membrane protein (Killer cell lectin-like | P26718 |
| | receptor subfamily K member 1, NK cell receptor D, NKG2-D- | |
| | activating NK receptor, CD314) | |
| OX40 | Tumor necrosis factor receptor superfamily member 4 (ACT35 | P43489 |
| | antigen, AX transcriptionally-activated glycoprotein 1 receptor) | |
| PD-1 | Programmed cell death protein 1 (CD279) | Q15116 |
| TIGIT | T-cell immunoreceptor with Ig and ITIM domains (V-set and | Q495A1 |
| | immunoglobulin domain-containing protein 9) (V-set and | |
| | transmembrane domain-containing protein 3) | |
| Tim-1 | Hepatitis A virus cellular receptor 1 (T-cell immunoglobulin and | Q96D42 |
| | mucin domain-containing protein 1, Kidney injury molecule 1, KIM-1, | |
| | T-cell immunoglobulin mucin receptor 1, T-cell membrane protein 1, | |
| | CD365) | |
| TIM3 | Hepatitis A virus cellular receptor 2, HAVcr-2 (T-cell immunoglobulin | Q8TDQ0 |
| | and mucin domain-containing protein 3, TIMD-3) (T-cell | |
| | immunoglobulin mucin receptor 3, TIM-3) (T-cell membrane protein | |
| | 3) | |
| CXCR3 | C-X-C chemokine receptor type 3 or CXC-R3 or CXCR-3 or CKR-L2 or G | P49682 |
| | protein-coupled receptor 9 or Interferon-inducible protein 10 | |
| | receptor (IP-10 receptor) or CD183 | |
| CXCR5 | C-X-C chemokine receptor type 5 or Burkitt lymphoma receptor 1 or | P32302 |
| | Monocyte-derived receptor 15 (MDR-15) or CD185 | |
| CRTAM | Cytotoxic and regulatory T-cell molecule, Class-I MHC-restricted T- | 095727 |
| | cell-associated molecule, CD355 | |
| CD226 | CD226 antigen, DNAX accessory molecule 1 (DNAM-1) | Q15762 |
| CD112R | Nectin-2, Nectin cell adhesion molecule 2 | Q92692 |
| CD27 | T-cell activation antigen CD27, T14, | P26842 |
| | Tumor necrosis factor receptor superfamily member 7 | |
| CD122 | Interleukin-2 receptor subunit beta, High affinity IL-2 receptor | P14784 |
| | subunit beta | |
| NGK2A | Natural killer cells antigen CD94, KP43, Killer cell lectin-like receptor | Q13241 |
| | subfamily D member 1, NK cell receptor | |
| CD103 | Integrin alpha-E, HML-1 antigen, Integrin alpha-IEL, Mucosal | P38570 |
| | lymphocyte 1 antigen | |
| CEACAM | Carcinoembryonic antigen-related cell adhesion molecule 1, Biliary | P13688 |
| | glycoprotein 1, CD66a | |

In a particular aspect, the T cell is an exhausted T cell or an effector memory stem like T cell and the target is a factor expressed on the surface of exhausted T cells or effector memory stem like T cells. T cell exhaustion is a state of T cell progressive loss of function, proliferation capacity and cytotoxic potential, eventually leading to their deletion. T cell exhaustion can be triggered by several factors such as persistent antigen exposure or inhibitory receptors including PD-1, TIM3, CTLA-4, LAG-3, BTLA, TIGIT and CD160. Preferably, such factor, in particular such exhaustion factor, is selected from the group consisting of PD-1, TIM3, CTLA-4, LAG3, BTLA, TIGIT and CD160.

In a preferred aspect, the first binding moiety is an antigen binding domain that has an antagonist activity on the target, in particular for checkpoint inhibitors between tumor cells and T cells (for example PD-1).

The term “antagonist” as used herein, refers to a substance that block or reduces the activity or functionality of another substance. Particularly, this term refers to a binding domain that binds to a cellular receptor (e.g., PD-1) as a reference substance (e.g., PD-L1 and/or PD-L2), preventing it from producing all or part of its usual biological effects (e.g., the creation of an immune suppressive microenvironment). The antagonist activity may be assessed by competitive ELISA.

In an embodiment, the first binding moiety is an antigen binding domain, in particular derived from an antibody. This means in particular that when it comes to antibody fragments such as a F(ab′)2, a Fab, a Fab′ or a scFV, such fragments, in particular VL and VH chains are thus from an antibody, in particular an antibody that has already been described in the art.

In a preferred aspect, the first binding moiety of the multifunctional molecule is an antibody, a fragment or a derivative thereof that is specific to an immune checkpoint inhibitor.

Even more preferably, the multifunctional molecule of the invention comprises a first binding moiety, said first binding moiety binding to a target specifically expressed on T cells surface selected from the group consisting of PD-1, VISTA, CTLA-4, BTLA, TIGIT, CD160, LAG3 and TIM3.

In a preferred aspect, the first binding moiety of the multifunctional molecule is an antibody, a fragment or a derivative thereof such as a F(ab′)2, a Fab, a Fab′ or a single-chain variable fragment (scFV) that is specific to PD-1, VISTA, CTLA-4, BTLA, TIGIT, CD160, LAG3 and TIM3.

Preferably, the first binding moiety of the multifunctional molecule is a F(ab′)2, a Fab, a Fab′ or a CrossMAb that is specific to PD-1, VISTA, CTLA-4, BTLA, TIGIT, CD160, LAG3 and TIM3, preferably to PD-1. Even more preferably, the first binding moiety of the multifunctional molecule is a Fab or a CrossMAb that is specific to PD-1, VISTA, CTLA-4, BTLA, TIGIT, CD160, LAG3 and TIM3, preferably to PD-1.

In a preferred aspect, the first binding moiety of the multifunctional molecule is from or is derived from an antibody, a fragment or a derivative thereof such as a F(ab′)2, a Fab, a Fab′ or a single-chain variable fragment (scFV) that is specific to PD-1, VISTA, CTLA-4, BTLA, TIGIT, CD160, LAG3 and TIM3.

This means that the first binding moiety comprises at least the antigen binding domain (i.e., the CDRs or the VH and VL domains of said antibody).

Numerous antibodies directed against PD-1, TIM3, VISTA, CTLA-4, LAG-3, BTLA, TIGIT and CD160 have already been described in the art.

As used herein, the terms “Programmed Death 1”, “Programmed Cell Death 1”, “PD1”, “PD-1”, “PDCD1”, “PD-1 antigen”, “human PD-1”, “hPD-1” and “hPD1” are used interchangeably and refer to the Programmed Death-1 receptor, also known as CD279, and include variants and isoforms of human PD-1, and analogs having at least one common epitope with PD-1. PD-1 is a key regulator of the threshold of immune response and peripheral immune tolerance. It is expressed on activated T cells, B cells, monocytes, and dendritic cells and binds to its ligands PD-L1 and PD-L2. Human PD-1 is encoded by the PDCD1 gene. As an example, the amino acid sequence of a human PD-1 is disclosed under GenBank accession number NP_005009. PD1 has four splice variants expressed on human Peripheral blood mononuclear cells (PBMC). Accordingly, PD-1 proteins include full-length PD-1, as well as alternative splice variants of PD-1, such as PD-1Aex2, PD-1Aex3, PD-1Aex2,3 and PD-1Aex2,3,4. Unless specified otherwise, the terms include any variant and, isoform of human PD-1 that are naturally expressed by PBMC, or that are expressed by cells transfected with a PD-1 gene.

Several anti-PD-1 are already clinically approved, and others are still in clinical developments. For instance, the anti-PD1 antibody can be selected from the group consisting of Pembrolizumab (also known as Keytruda, lambrolizumab, MK-3475, SCH-900475), Nivolumab (OPDIVO, MDX-1106, BMS-936558, ONO-4538), Pidilizumab (CT-011), Cemiplimab (Libtayo), Camrelizumab, AUNP12, AMP-224, AGEN-2034 (Balstilimab), BGB-A317 (Tisleizumab), PDR001 (spartalizumab), MK-3477, PF-06801591 (Sasanlimab), JNJ-63723283 (Cetrelimab), genolimzumab (CBT-501), LZM-009 (Lipustobart), BCD-100 (Prolgolimab), SHR-1201, BAT-1306, AK-103 (HX-008), MEDI-0680 (also known as AMP-514), JS001 (see Si-Yang Liu et al., J. Hematol. Oncol. 10:136 (2017)), BI-754091, CBT-501, INCSHR1210 (also known as SHR-1210), TSR-042 (also known as ANB011), GLS-010 (also known as WBP3055), AM-0001 (Armo), STI-1110 (see WO 2014/194302), MGA012 (see WO 2017/19846), or IBI308 (see WO 2017/024465, WO 2017/025016, WO 2017/132825, and WO 2017/133540, the disclosure thereof being incorporated herein by reference), monoclonal antibodies 5C4, 17D8, 2D3, 4H1, 4A11, 7D3, and 5F4, described in WO 2006/121168, the disclosure thereof being incorporated herein by reference. Multifunctional molecules targeting PD-1 are also known such as RG7769 (Roche), XmAb20717 (Xencor), MED15752 (AstraZeneca), FS118 (F-star), SL-279252 (Takeda) and XmAb23104 (Xencor).

In some aspects, the target is PD-1 and the first binding moiety is specific to PD-1. Preferably, the first binding moiety is an antagonist of PD-1. Even more preferably, the anti-PD-1 antibody is Pembrolizumab, Nivolumab or OSE-279 (such as described in WO2020/127366, the disclosure thereof being incorporated herein by reference).

In a particular aspect, the anti-PD1 antibody can be Pembrolizumab (also known as Keytruda lambrolizumab, MK-3475) or Nivolumab (Opdivo, MDX-1106, BMS-936558, ONO-4538).

Antibodies directed against TIM3 and multifunctional molecules targeting TIM3 are also known such as Sym023, TSR-022, MBG453, LY3321367, INCAGNO2390, BGTB-A425, LY3321367, RG7769 (Roche).

In some aspects, a TFM-3 antibody is as disclosed in International Patent Application Publication Nos. WO2013006490, WO2016/161270, WO 2018/085469, or WO 2018/129553, WO 2011/155607, U.S. Pat. No. 8,552,156, EP 2581113 and U.S 2014/044728, the disclosure thereof being incorporated herein by reference.

Antibodies directed against CTLA-4 and multifunctional molecules targeting CTLA-4 are also known such as ipilimumab, tremelimumab, MK-1308, AGEN-1884, XmAb20717 (Xencor), MED15752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WOO9100140 and WO07008463, the disclosure thereof being incorporated herein by reference.

Antibodies directed against LAG3 and multifunctional molecules targeting LAG-3 are also known such as BMS-986016, IMP701, MGDO12 or MGDO13 (bispecific PD-1 and LAG-3 antibody). Anti-LAG-3 antibodies are also disclosed in WO2008132601, EP2320940, WO19152574, the disclosure thereof being incorporated herein by reference.

Antibodies directed against BTLA are also known in the art such as hu Mab8D5, hu Mab8A3, hu Mab21H6, hu Mab19A7, or hu Mab4C7. The antibody TABOO4 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3), the disclosure thereof being incorporated herein by reference.

Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484. Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728, the disclosure thereof being incorporated herein by reference.

Antibodies directed against CD160 are also known in the art, such as CL1-R2 CNCM I-3204 as disclosed in WO06015886, or others as disclosed in WO10006071, WO10084158, WO18077926, the disclosure thereof being incorporated herein by reference.

In another particular aspect, the target is PD-1 and the first binding moiety of the multifunctional molecule is the antigen binding domain, in particular an antibody, a fragment or a derivative thereof such as a Fab or a scFv or an antibody mimic, that is specific to PD-1. Then, in a particular aspect, the antigen binding domain comprised in the multifunctional molecule according to the invention is an anti-PD1 antibody or antigen binding fragment thereof, preferably a human, humanized or chimeric anti-PD1 antibody or antigen binding fragment thereof.

In an embodiment, the target is PD-1 and the first binding moiety a F(ab′)2, a Fab or a scFv that is specific to PD-1, preferably a human, humanized or chimeric anti-PD1 Fab or scFv.

Preferably, the antigen binding domain is an antagonist of PD-1.

In a very specific aspect of the present disclosure, the first binding moiety is an antigen binding domain that targets PD-1 and is derived from the antibody disclosed in WO2020/127366, the disclosure thereof being incorporated herein by reference.

Then, in an embodiment, the first binding moiety comprises an anti-PD-1 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 1, optionally with one, two or three modification(s) selected from substitution(s), addition(s), deletion(s) and any combination thereof, in particular at any position but position 3 of SEQ ID NO: 1;
   the heavy chain CDR2 (HCDR2) comprises or consists of an amino acid sequence of SEQ ID NO: 2, optionally with one, two or three modification(s) selected from substitution(s), addition(s), deletion(s) and any combination thereof, in particular at any position but positions 13, 14 and 16 of SEQ ID NO: 2;
   the heavy chain CDR3 (HCDR3) comprises or consists of an amino acid sequence of SEQ ID NO: 3;
   optionally with one, two or three modification(s) selected from substitution(s), addition(s), deletion(s) and any combination thereof, in particular at any position but positions 2, 3, 7 and 8 of SEQ ID NO: 3;
   the light chain CDR1 (LCDR1) comprises or consists of an amino acid sequence of SEQ ID NO: 4, optionally with one, two or three modification(s) selected from substitution(s), addition(s), deletion(s) and any combination thereof, in particular at any position but positions 5, 6, 10, 11 and 16 of SEQ ID NO: 4;
   the light chain CDR2 (LCDR2) comprises or consists of an amino acid sequence of SEQ ID NO: 5, optionally with one, two or three modification(s) selected from substitution(s), addition(s), deletion(s) and any combination thereof; and
   the light chain CDR3 (LCDR3) comprises or consists of an amino acid sequence of SEQ ID NO: 6, optionally with one, two or three modification(s) selected from substitution(s), addition(s), deletion(s) and any combination thereof at any position but positions 1, 4 and 6 of SEQ ID NO: 6.

In another aspect, the anti-PD-1 antigen-binding domain comprises or consists essentially of: (i) a heavy chain variable region (VH) comprising a CDR1 of SEQ ID NO: 1, a CDR2 of SEQ ID NO: 2 and a CDR3 of SEQ ID NO: 3; and (ii) a light chain variable region (VL) comprising a CDR1 of SEQ ID NO: 4, a CDR2 of SEQ ID NO: 5 and a CDR3 of SEQ ID NO: 6.

Preferably, the CDRs of such anti-PD1 binding domain have been determined by the Kabat method.

In one aspect, the an anti-PD-1 antigen-binding domain comprises framework regions, in particular heavy chain variable region framework regions (HFR) HFR1, HFR2, HFR3 and HFR4 and light chain variable region framework regions (LFR) LFR1, LFR2, LFR3 and LFR4, especially HFR1, HFR2, HFR3 and HFR4, optionally with one, two or three modification(s) selected from substitution(s), addition(s), deletion(s) and any combination thereof, in particular at any position but positions 27, 29 and 32 of HFR3, i.e., of SEQ ID NO: 9. Preferably, the anti-PD-1 antigen-binding domain comprises HFR1 of SEQ ID NO: 7, HFR2 of SEQ ID NO: 8, HFR3 of SEQ ID NO: 9 and HFR4 of SEQ ID NO: 10. In addition, the anti-PD-1 antigen-binding domain may comprise light chain variable region framework regions (LFR) LFR1, LFR2, LFR3 and LFR4, optionally with one, two or three modification(s) selected from substitution(s), addition(s), deletion(s) and any combination thereof. Preferably, the anti-PD-1 antigen-binding domain comprises LFR1 of SEQ ID NO: 11, LFR2 of SEQ ID NO: 12, LFR3 of SEQ ID NO: 13 and LFR4 of SEQ ID NO: 14.

In an aspect, the anti-PD1 antigen-binding domain comprises or consists essentially of:
(a) a heavy chain variable region (VH) comprising or consisting of an amino acid sequence of SEQ ID NO: 15, optionally with one, two or three modification(s) selected from substitution(s), addition(s), deletion(s) and any combination thereof, in particular at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 15;
(b) a light chain variable region (VL) comprising or consisting of an amino acid sequence of SEQ ID NO: 16, optionally with one, two or three modification(s) selected from substitution(s), addition(s), deletion(s) and any combination thereof, in particular at any position but positions 3, 4, 7, 14, 17, 18, 28, 29, 33, 34, 39, 42, 44, 50, 81, 88, 94, 97, 99 and 105 of SEQ ID NO: 16.

Preferably, the one, two or three modification(s) are outside of the CDRs (i.e., are in the framework regions).

In an aspect, the anti-PD1 antigen-binding domain comprises or consists essentially of:
(a) a heavy chain variable region (VH) comprising or consisting of an amino acid sequence as set forth in SEQ ID NO: 15 or a sequence having 80%, 85%, 90%, 95%, 97% or 99% sequence identity thereto; and
(b) a light chain variable region (VL) comprising or consisting of an amino acid sequence as set forth in SEQ ID NO: 16 or a sequence having 80%, 85%, 90%, 95%, 97% or 99% sequence identity thereto.

In another aspect, the anti-PD1 antigen-binding domain comprises or consists essentially of:
(a) a heavy chain variable region (VH) comprising or consisting of an amino acid sequence of SEQ ID NO: 15; and (b) a light chain variable region (VL) comprising or consisting of an amino acid sequence of SEQ ID NO: 16.

In another embodiment, the first binding moiety comprises an anti-PD-1 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 65;
   the heavy chain CDR2 (HCDR2) comprises or consists of an amino acid sequence of SEQ ID NO: 66;
   the heavy chain CDR3 (HCDR3) comprises or consists of an amino acid sequence of SEQ ID NO: 67;
   the light chain CDR1 (LCDR1) comprises or consists of an amino acid sequence of SEQ ID NO: 68;
   the light chain CDR2 (LCDR2) comprises or consists of an amino acid sequence of SEQ ID NO: 69, and
   the light chain CDR3 (LCDR3) comprises or consists of an amino acid sequence of SEQ ID NO: 70.

Preferably, said anti-PD-1 antigen-binding domain comprises i) a VH comprising or consisting of an amino acid sequence of SEQ ID NO: 71; and ii) a VL comprising or consisting of an amino acid sequence of SEQ ID NO: 72.

In another embodiment, the first binding moiety comprises an anti-PD-1 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 73;
   the heavy chain CDR2 (HCDR2) comprises or consists of an amino acid sequence of SEQ ID NO: 74;
   the heavy chain CDR3 (HCDR3) comprises or consists of an amino acid sequence of SEQ ID NO: 75;
   the light chain CDR1 (LCDR1) comprises or consists of an amino acid sequence of SEQ ID NO: 76;
   the light chain CDR2 (LCDR2) comprises or consists of an amino acid sequence of SEQ ID NO: 77, and
   the light chain CDR3 (LCDR3) comprises or consists of an amino acid sequence of SEQ ID NO: 78.

Preferably, said anti-PD-1 antigen-binding domain comprises i) a VH comprising or consisting of an amino acid sequence of SEQ ID NO: 79; and ii) a VL comprising or consisting of an amino acid sequence of SEQ ID NO: 80.

In an embodiment, the anti-PD-1 antigen-binding domain comprises VH, VL, CH1 and CL domains, so that the antigen binding domain is a Fab.

In such embodiment, the heavy chain constant domain (CH1) comprises or consists essentially of SEQ ID NO: 17, optionally with one, two or three modification(s) selected from substitution(s), addition(s), deletion(s) and any combination thereof.

Preferably, the light chain constant domain (CL) comprises or consists essentially of SEQ ID NO: 18 optionally with one, two or three modification(s) selected from substitution(s), addition(s), deletion(s) and any combination thereof.

In an embodiment, the anti-PD-1 antigen-binding domain is a Fab or a Fab′, a Fab or a F(ab′)2 and comprises i) a VH domain and a CH1 domain, said VH and CH1 domains having the amino acid sequence as set forth in SEQ ID NOs: 15 and 16, respectively, optionally with one, two or three modification(s) selected from substitution(s), addition(s), deletion(s) and any combination thereof; and ii) a VL domain and a CL domain, id domains having the amino acid sequence as set forth in SEQ ID NOs: 16 and 18, respectively, optionally with one, two or three modification(s) selected from substitution(s), addition(s), deletion(s) and any combination thereof.

Preferably, the first binding moiety is an anti-PD-1 Fab or F(ab′)2, comprising or consisting of i) a chain comprising or consisting of a VH domain and a CH1 domain, said VH and CH1 domains having the amino acid sequence as set forth in SEQ ID NOs: 15 and 16 respectively and ii) a chain comprising or consisting of VL and CL domains, said domains having the amino acid sequence as set forth in SEQ ID NOs: 16 and 18, respectively.

In an embodiment, the first binding moiety is an anti-PD-1 Fab or F(ab′)2, comprising or consisting of of i) a chain comprising or consisting of an amino acid sequence as set forth in SEQ ID NO: 19 optionally with one, two or three modification(s) selected from substitution(s), addition(s), deletion(s) and any combination thereof and of ii) a chain comprising or consisting of an amino acid sequence as set forth in SEQ ID NO: 20 optionally with one, two or three modification(s) selected from substitution(s), addition(s), deletion(s) and any combination thereof.

In an embodiment, the first binding moiety comprises an anti-PD1 CrossMAb comprising or consisting of i) a chain comprising or consisting of VH and CL domains, said domains having the amino acid sequence as set forth in SEQ ID NOs: 15 and 18, respectively, optionally with one, two or three modification(s) selected from substitution(s), addition(s), deletion(s) and any combination thereof; ii) a chain comprising or consisting of VL and CH1 domains, said domains having the amino acid sequence as set forth in SEQ ID NOs: 16 and 17, respectively, optionally with one, two or three modification(s) selected from substitution(s), addition(s), deletion(s) and any combination thereof.

Preferably, the first binding moiety comprises an anti-PD1 CrossMAb, comprising or consisting of i) a chain comprising or consisting of a VH domain and a CL domain, said domains having the amino acid sequence as set forth in SEQ ID NOs: 15 and 18, respectively, and ii) a chain comprising or consisting of VL and CH1 domains, said domains having the amino acid sequence as set forth in SEQ ID NOs: 16 and 17, respectively.

In an embodiment, the first binding moiety is an anti-PD-1 CrossMAb, comprising or consisting of i) a chain comprising or consisting of an amino acid sequence as set forth in SEQ ID NO: 21 optionally with one, two or three modification(s) selected from substitution(s), addition(s), deletion(s) and any combination thereof and of ii) a chain comprising or consisting of an amino acid sequence as set forth in SEQ ID NO: 22 optionally with one, two or three modification(s) selected from substitution(s), addition(s), deletion(s) and any combination thereof.

Preferably, the one, two or three modification(s) in the VH and/or VL or CH and/or CL are outside of the CDRs (i.e., are in the framework regions) for any one the VH, VL, CH and/or CL described here above.

### Second Binding Moiety

The multifunctional molecule according to the invention comprise a second binding moiety, said second binding moiety being an agonist of CD28, and being an anti-CD28 antigen binding domain. The multifunctional molecule comprises a single second binding moiety so that the molecule comprises only one antigen binding domain that binds to CD28. In particular, the multifunctional molecule is monovalent for the targeting of CD28 (has only on valency against CD28).

As used herein, “CD28” relates to a co-stimulatory molecule expressed on the surface of several immune cells such as T cells. According to a specific embodiment, the CD28 protein refers to the human protein, such as provided in the following GenBank Numbers NP_001230006, NP_001230007, NP_006130. Human-CD28 cDNA is freely available from Dr. A. Aruffo and Dr. B. Seed, who published the sequence and also the following part of literature: Aruffo, A., and Seed, B., 1987, “Molecular cloning of a CD28 cDNA by a high efficiency COS cell expression system”, Proc. Natl. Acad. Sci. USA, 84:8573. Therefore, the production of the human CD28 cDNA can be seen in detail from this literature. Several ligands for CD28 have been identified, B7.1 (also known as CD80) and B7.2 (also known as CD86). The B7.1 protein particularly refers to the human protein, such as provided in the following GenBank Number NP_005182. According to a specific embodiment, the B7.2 protein refers to the human protein, such as provided in the following GenBank Number NP_001193853.

The interaction of CD28 with its ligand triggers a co-stimulatory signal that synergizes with the TCR signal to promote T-cell activation, proliferation and function. CD28 signaling was shown to regulate the threshold for T-cell activation and decrease the number of TCR engagements needed for T-cell activation.

As used herein, the term “CD28 agonist” refers to an agonistic agent that induces and/or increases the biological function and/or expression of CD28. According to specific embodiments, the CD28 agonist induces and/or increases signaling to an immune cell (e.g., T cells) by CD28; thereby induces and/or increases CD28 immune co-stimulatory activity. In particular, the CD28 agonist promotes immune response of an effector T cell following TCR activating signal.

According to specific embodiments, the CD28 agonist binds directly to CD28 and activates the CD28 receptor.

In certain embodiments, the CD28 agonist exhibits one or more desirable functional properties, such as high affinity binding to CD28, e.g., binding to human CD28 with a KD of 10⁷ M, 10⁸ M, 10⁹ M, 10¹⁰ M or less; lack of significant cross-reactivity to other immune-check point proteins, e.g., CTLA-4 and ICOS; the ability to stimulate T cell proliferation; the ability to increase IFN-γ and/or IL-2 secretion; the ability to stimulate antigen-specific memory responses; the ability to stimulate antibody responses and/or the ability to inhibit growth of tumor cells.

In some embodiments, the CD28 agonist is a Fab, a Fab′, a single-chain variable fragment (scFV) or a single domain antibody (sdAb), preferably a Fab or a scFV that derives from an antibody, in particular a super agonistic anti-CD28 antibody or a conventional anti-CD28 antibody.

In some instances, the anti-CD28 binding domain is not a sdAb or a VHH.

As used herein, the terms “conventional anti-CD28 antibody” refers to an antibody which binds CD28 (e.g., in a domain outside the basolateral domain) and co-stimulates T cells in a TCR-dependent mechanism.

As used herein, the terms “super agonistic anti-CD28 antibody” refers to an antibody which binds CD28 through the basolateral domain resulting in a polyclonal activation of T lymphocytes even in the absence of TCR stimulation.

Anti-CD28 antibodies suitable for use in the invention can be generated using methods well known in the art. Alternatively, art recognized anti-CD28 antibodies can be used. Examples of anti-CD28 antibodies are disclosed for example in Poirier et al. (2012) American Journal of Transplantation 12(7): 1682-1690, Cell Immunol. 2005 July-August; 236(1-2): 154-60, which are hereby incorporated by reference in their entirety.

Specific anti-CD28 antibodies that can be used according to some embodiments of the present invention include, but are not limited to TAB08 (previously known as TGN1412, produced by TheraMAB), a humanized monoclonal antibody directed against human CD28, anti-CD28 Agonist Antibody (Humanized) provided by Bioscience Catalog #: 100186 or mouse monoclonal antibody directed against human CD28 (clone CD28.2, provided for example by eBioscience #16-0289-81).

Then, in an embodiment, the second binding moiety comprises an anti-CD28 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 30, optionally with one, two or three modification(s) selected from substitution(s), addition(s), deletion(s) and any combination thereof;
   the heavy chain CDR2 (HCDR2) comprises or consists of an amino acid sequence of SEQ ID NO: 31, optionally with one, two or three modification(s) selected from substitution(s), addition(s), deletion(s) and any combination thereof;
   the heavy chain CDR3 (HCDR3) comprises or consists of an amino acid sequence of SEQ ID NO: 32;
   optionally with one, two or three modification(s) selected from substitution(s), addition(s), deletion(s) and any combination thereof;
   the light chain CDR1 (LCDR1) comprises or consists of an amino acid sequence of SEQ ID NO: 33, optionally with one, two or three modification(s) selected from substitution(s), addition(s), deletion(s) and any combination thereof;
   the light chain CDR2 (LCDR2) comprises or consists of an amino acid sequence of SEQ ID NO: 34, optionally with one, two or three modification(s) selected from substitution(s), addition(s), deletion(s) and any combination thereof; and
   the light chain CDR3 (LCDR3) comprises or consists of an amino acid sequence of SEQ ID NO: 35, optionally with one, two or three modification(s) selected from substitution(s), addition(s), deletion(s) and any combination thereof.

In another aspect, the second antigen-binding domain comprises or consists essentially of: (i) a heavy chain variable region (VH) comprising a CDR1 of SEQ ID NO: 30, a CDR2 of SEQ ID NO: 31 and a CDR3 of SEQ ID NO: 32; and (ii) a light chain variable region (VL) comprising a CDR1 of SEQ ID NO: 33 a CDR2 of SEQ ID NO: 34 and a CDR3 of SEQ ID NO: 35.

Preferably, the CDRs of such anti-CD28 binding domain have been determined by the Kabat method.

In one aspect, such anti-CD28 antigen-binding domain comprises framework regions, in particular heavy chain variable region framework regions (HFR) HFR1, HFR2, HFR3 and HFR4 and light chain variable region framework regions (LFR) LFR1, LFR2, LFR3 and LFR4. Preferably, the anti-CD28 antigen-binding domain comprises HFR1 of SEQ ID NO: 36, HFR2 of SEQ ID NO: 37, HFR3 of SEQ ID NO: 38 and HFR4 of SEQ ID NO: 39 optionally with one, two or three modification(s) selected from substitution(s), addition(s), deletion(s) and any combination thereof. In addition, the anti-CD28 antigen-binding domain may comprises LFR1 of SEQ ID NO: 40, LFR2 of SEQ ID NO: 41, LFR3 of SEQ ID NO: 42 and LFR4 of SEQ ID NO: 43 optionally with one, two or three modification(s) selected from substitution(s), addition(s), deletion(s) and any combination thereof.

The framework regions and complementary determining regions are preferably operably linked in the following order: FR1-CDR1-FR2-CDR2-FR3-CDR3-FR4 (from amino terminus to carboxy terminus). In another embodiment, the second binding moiety comprises an anti-CD28 antigen-binding domain comprising:
(i) a heavy chain variable domain comprising HCDR1, HCDR2 and HCDR3, and
(ii) a light chain variable domain comprising LCDR1, LCDR2 and LCDR3,
wherein:
   the heavy chain CDR1 (HCDR1) comprises or consists of an amino acid sequence of SEQ ID NO: 91, optionally with one, two or three modification(s) selected from substitution(s), addition(s), deletion(s) and any combination thereof;
   the heavy chain CDR2 (HCDR2) comprises or consists of an amino acid sequence of SEQ ID NO: 92, optionally with one, two or three modification(s) selected from substitution(s), addition(s), deletion(s) and any combination thereof;
   the heavy chain CDR3 (HCDR3) comprises or consists of an amino acid sequence of SEQ ID NO: 93; optionally with one, two or three modification(s) selected from substitution(s), addition(s), deletion(s) and any combination thereof;
   the light chain CDR1 (LCDR1) comprises or consists of an amino acid sequence of SEQ ID NO: 94, optionally with one, two or three modification(s) selected from substitution(s), addition(s), deletion(s) and any combination thereof;
   the light chain CDR2 (LCDR2) comprises or consists of an amino acid sequence of SEQ ID NO: 95, optionally with one, two or three modification(s) selected from substitution(s), addition(s), deletion(s) and any combination thereof; and
   the light chain CDR3 (LCDR3) comprises or consists of an amino acid sequence of SEQ ID NO: 96, optionally with one, two or three modification(s) selected from substitution(s), addition(s), deletion(s) and any combination thereof.

In another aspect, the second antigen-binding domain comprises or consists essentially of: (i) a heavy chain variable region (VH) comprising a CDR1 of SEQ ID NO: 91, a CDR2 of SEQ ID NO: 92 and a CDR3 of SEQ ID NO: 93; and (ii) a light chain variable region (VL) comprising a CDR1 of SEQ ID NO: 94, a CDR2 of SEQ ID NO: 96 and a CDR3 of SEQ ID NO: 97.

Preferably, the CDRs of such anti-CD28 binding domain have been determined by the IMGT method.

In one aspect, the anti-CD28 antigen-binding domain comprises framework regions, in particular heavy chain variable region framework regions (HFR) HFR1, HFR2, HFR3 and HFR4 and light chain variable region framework regions (LFR) LFR1, LFR2, LFR3 and LFR4. Preferably, the anti-CD28 antigen-binding domain comprises HFR1 of SEQ ID NO: 97, HFR2 of SEQ ID NO: 98, HFR3 of SEQ ID NO: 99 and HFR4 of SEQ ID NO: 100 optionally with one, two or three modification(s) selected from substitution(s), addition(s), deletion(s) and any combination thereof. In addition, the anti-CD28 antigen-binding domain may comprises LFR1 of SEQ ID NO: 101, LFR2 of SEQ ID NO: 102, LFR3 of SEQ ID NO: 103 and LFR4 of SEQ ID NO: 104 optionally with one, two or three modification(s) selected from substitution(s), addition(s), deletion(s) and any combination thereof.

These framework regions and complementary determining regions are preferably operably linked in the following order: FR1-CDR1-FR2-CDR2-FR3-CDR3-FR4 (from amino terminus to carboxy terminus).

In an aspect, the anti-CD28 antigen-binding domain comprises or consists essentially of:
(a) a heavy chain variable region (VH) comprising or consisting of an amino acid sequence SEQ ID NO: 44, optionally with one, two or three modification(s) selected from substitution(s), addition(s), deletion(s) and any combination thereof;
(b) a light chain variable region (VL) comprising or consisting of an amino acid sequence of SEQ ID NO: 45, optionally with one, two or three modification(s) selected from substitution(s), addition(s), deletion(s) and any combination thereof.

In another aspect, the anti-CD28 antigen-binding domain comprises or consists essentially of:
(a) a heavy chain variable region (VH) comprising or consisting of an amino acid sequence of SEQ ID NO: 44; and (b) a light chain variable region (VL) comprising or consisting of an amino acid sequence of SEQ ID NO: 45.

Preferably, the anti-CD28 antigen-binding domain is a scFV that comprises or consists essentially of a heavy chain variable region (VH) of SEQ ID NO: 44 and a light chain variable region (VL) of SEQ ID NO: 5, optionally linked by a peptide linker.

In an embodiment, the anti-CD28 antigen-binding domain comprises VH, VL, CH1 and CL domains, so that the antigen binding domain is a Fab.

In such embodiment, the heavy chain constant domain (CH1) preferably comprises or consists essentially of SEQ ID NO: 46, optionally with one, two or three modification(s) selected from substitution(s), addition(s), deletion(s) and any combination thereof.

Preferably, the light chain constant domain (CL) comprises or consists essentially of SEQ ID NO: 47 optionally with one, two or three modification(s) selected from substitution(s), addition(s), deletion(s) and any combination thereof.

In an embodiment, the anti-CD28 binding moiety is a Fab comprising or consisting of i) a chain comprising or consisting of a VH domain and a CH1 domain, said domains having the amino acid sequence as set forth in SEQ ID NOs: 44 and 46, respectively, optionally with one, two or three modification(s) selected from substitution(s), addition(s), deletion(s) and any combination thereof; ii) a light chain comprising or consisting of VL and CL domains, said domains having the amino acid sequence as set forth in SEQ ID NOs: 45 and 47, respectively, optionally with one, two or three modification(s) selected from substitution(s), addition(s), deletion(s) and any combination thereof.

In an embodiment, the anti-CD28 binding moiety is a Fab comprising or consisting of i) a chain comprising or consisting of an amino acid sequence as set forth in SEQ ID NO: 48 optionally with one, two or three modification(s) selected from substitution(s), addition(s), deletion(s) and any combination thereof and of ii) a chain comprising or consisting of an amino acid sequence as set forth in SEQ ID NO: 49 optionally with one, two or three modification(s) selected from substitution(s), addition(s), deletion(s) and any combination thereof.

In an embodiment, the anti-CD28 binding moiety is a CrossMAb comprising or consisting of i) a chain comprising or consisting of a VH domain and a CL domain, said domains having the amino acid sequence as set forth in SEQ ID NOs: 44 and 47, respectively, optionally with one, two or three modification(s) selected from substitution(s), addition(s), deletion(s) and any combination thereof; ii) a light chain comprising or consisting of VL and CH1 domains, said domains having the amino acid sequence as set forth in SEQ ID NOs: 45 and 46, respectively, optionally with one, two or three modification(s) selected from substitution(s), addition(s), deletion(s) and any combination thereof.

Preferably, the anti-CD28 binding moiety comprises a CrossMAb, comprising or consisting of i) a chain comprising or consisting of a VH domain and a CL domain, said domains having the amino acid sequence as set forth in SEQ ID NOs: 44 and 47, respectively, and ii) a light chain comprising or consisting of VL and CH1 domains, said domains having the amino acid sequence as set forth in SEQ ID NOs: 45 and 46, respectively.

In an embodiment, the anti-CD28 binding moiety is a Fab comprising or consisting of i) a chain comprising or consisting of an amino acid sequence as set forth in SEQ ID NO: 50 optionally with one, two or three modification(s) selected from substitution(s), addition(s), deletion(s) and any combination thereof and of ii) a chain comprising or consisting of an amino acid sequence as set forth in SEQ ID NO: 51 optionally with one, two or three modification(s) selected from substitution(s), addition(s), deletion(s) and any combination thereof.

Preferably, the one, two or three modification(s) in the VH and/or VL or CH and/or CL are outside of the CDRs (i.e., are in the framework regions) for any one the VH, VL, CH and/or CL described here above.

In some embodiments, the anti-CD28 binding domain is a variant comprising one or more mutations in the CDRs and/or framework regions compared to a reference sequence. In particular, said variant is a function conservative variant. In the context on the present invention, this means that said variant i) binds to CD28, ii) has an agonist activity on CD28 and iii) when comprised in a multifunctional molecule of the invention, is able to confer cis-activity properties to the multifunctional molecule. Methods to select suitable multifunctional molecule comprising such variant are for example disclose in the section “Method of selecting a suitable multifunctional molecule” provided below.

Particularly, the anti-CD28 binding domain comprises or consists essentially of:
(i) a heavy chain variable region (VH) comprising:
a HFR1 of SEQ ID NO: 36; optionally with 1, 2, 3, 4, 5, 6, 7, 8, 9, 10 or 11 modification(s) selected from substitution(s), addition(s), deletion(s) and any combination thereof, preferably amino acid substitutions;
a HCDR1 of SEQ ID NO: 30, optionally with 1, 2 or 3 modification(s) selected from substitution(s), addition(s), deletion(s) and any combination thereof, preferably amino acid substitutions;
a HFR2 of SEQ ID NO: 37, optionally with 1, 2 or 3 modification(s) selected from substitution(s), addition(s), deletion(s) and any combination thereof, preferably amino acid substitutions;
a HCDR2 of SEQ ID NO: 31, optionally with 1, 2 or 3 modification(s) selected from substitution(s), addition(s), deletion(s) and any combination thereof, preferably amino acid substitutions;
a HFR3 of SEQ ID NO: 38, optionally with 1, 2, 3, 4, 5, 6 or 7 modification(s) selected from substitution(s), addition(s), deletion(s) and any combination thereof, preferably amino acid substitutions;
a HCDR3 of SEQ ID NO: 32; optionally with 1, 2 or 3 modification(s) selected from substitution(s), addition(s), deletion(s) and any combination thereof, preferably amino acid substitutions; and
a HFR4 of SEQ ID NO: 39, optionally with 1, 2 or 3 modification(s) selected from substitution(s), addition(s), deletion(s) and any combination thereof preferably amino acid substitutions;
and (ii) a light chain variable region (VL) comprising:
a LFR1 of SEQ ID NO: 40, optionally with 1, 2, 3, 4, 5, 6 or 7 modification(s) selected from substitution(s), addition(s), deletion(s) and any combination thereof, preferably amino acid substitutions;
a LCDR1 of SEQ ID NO: 33, optionally with 1, 2 or 3 modification(s) selected from substitution(s), addition(s), deletion(s) and any combination thereof, preferably amino acid substitutions;
a LFR2 of SEQ ID NO: 41, optionally with 1, 2 or 3 modification(s) selected from substitution(s), addition(s), deletion(s) and any combination thereof, preferably amino acid substitutions;
a LCDR2 of SEQ ID NO: 34, optionally with 1, 2 or 3 modification(s) selected from substitution(s), addition(s), deletion(s) and any combination thereof, preferably amino acid substitutions;
a LFR3 of SEQ ID NO: 42, optionally with 1, 2, 3, 4, 5, 6, 7, or 8 modification(s) selected from substitution(s), addition(s), deletion(s) and any combination thereof, preferably amino acid substitutions;
a LCDR3 of SEQ ID NO: 35, optionally with 1, 2 or 3 modification(s) selected from substitution(s), addition(s), deletion(s) and any combination thereof, preferably amino acid substitutions; and
a LFR4 of SEQ ID NO: 43, optionally with 1, 2 or 3 modification(s) selected from substitution(s), addition(s), deletion(s) and any combination thereof, preferably amino acid substitutions.

Preferably, the anti-CD28 binding domain comprises or consists essentially of:
(i) a heavy chain variable region (VH) comprising:
a HFR1 of SEQ ID NO: 36; optionally with 1, 2, 3, 4, 5, 6, 7, 8, 9, 10 or 11 amino acid modification(s) selected from substitution(s), addition(s), deletion(s) and any combination thereof, preferably amino acid substitutions;
a HCDR1 of SEQ ID NO: 30, optionally with 1 amino acid modification selected from substitution, addition, deletion, preferably an amino acid substitution;
a HFR2 of SEQ ID NO: 37, optionally with 1 or 2 amino acid modification(s) selected from substitution(s), addition(s), deletion(s) and any combination thereof, preferably amino acid substitutions;
a HCDR2 of SEQ ID NO: 31;
a HFR3 of SEQ ID NO: 38, optionally with 1, 2, 3, 4, 5, 6 or 7 amino acid modification(s) selected from substitution(s), addition(s), deletion(s) and any combination thereof, preferably amino acid substitutions;
a HCDR3 of SEQ ID NO: 32; and
a HFR4 of SEQ ID NO: 39, optionally with 1 amino acid modification selected from substitution, addition, deletion, preferably an amino acid substitution;
and (ii) a light chain variable region (VL) comprising:
a LFR1 of SEQ ID NO: 40, optionally with 1, 2, 3, 4, 5, 6 or 7 modification(s) selected from substitution(s), addition(s), deletion(s) and any combination thereof, preferably amino acid substitutions;
a LCDR1 of SEQ ID NO: 33, optionally with 1 amino acid modification selected from substitution, addition, deletion, preferably an amino acid substitution;
a LFR2 of SEQ ID NO: 41, optionally with 1, 2 or 3 modification(s) selected from substitution(s), addition(s), deletion(s) and any combination thereof, preferably amino acid substitutions;
a LCDR2 of SEQ ID NO: 34, optionally with 1 amino acid modification selected from substitution, addition, deletion, preferably an amino acid substitution;
a LFR3 of SEQ ID NO: 42, optionally with 1, 2, 3, 4, 5, 6, 7, or 8 modification(s) selected from substitution(s), addition(s), deletion(s) and any combination thereof, preferably amino acid substitutions;
a LCDR3 of SEQ ID NO: 35; and
a LFR4 of SEQ ID NO: 43, optionally with 1 amino acid modification selected from substitution, addition, deletion, preferably an amino acid substitution.

In another embodiment, the anti-CD28 binding domain comprises or consists essentially of:
(i) a heavy chain variable region (VH) comprising:
a HFR1 of SEQ ID NO: 36; optionally with 1, 2, 3, 4, 5, 6, 7, 8, 9, 10 or 11 amino acid modification(s) selected from substitution(s), addition(s), deletion(s) and any combination thereof, preferably amino acid substitutions;
a HCDR1 of SEQ ID NO: 30, optionally with 1 amino acid modification selected from substitution, addition, deletion, preferably an amino acid substitution;
a HFR2 of SEQ ID NO: 37, optionally with 1 or 2 amino acid modification(s) selected from substitution(s), addition(s), deletion(s) and any combination thereof, preferably amino acid substitutions;
a HCDR2 of SEQ ID NO: 31;
a HFR3 of SEQ ID NO: 38, optionally with 1, 2, 3, 4, 5, 6 or 7 amino acid modification(s) selected from substitution(s), addition(s), deletion(s) and any combination thereof, preferably amino acid substitutions;
a HCDR3 of SEQ ID NO: 32; and
a HFR4 of SEQ ID NO: 39, optionally with 1 amino acid modification selected from substitution, addition, deletion, preferably an amino acid substitution;
and (ii) a light chain variable region (VL) comprising or consisting of an amino acid sequence of SEQ ID NO: 45, optionally with 1, 2 or 3 modification(s) selected from substitution(s), addition(s), deletion(s) and any combination thereof, preferably amino acid substitutions; said modification(s) being preferably in the framework regions.

In another embodiment, the anti-CD28 binding domain comprises or consists essentially of:
(i) a heavy chain variable region (VH) comprising or consisting of an amino acid sequence of SEQ ID NO: 44, optionally with 1, 2 or 3 modification(s) selected from substitution(s), addition(s), deletion(s) and any combination thereof, preferably amino acid substitutions; said modification(s) being preferably in the framework regions; and
(ii) a light chain variable region (VL) comprising:
a LFR1 of SEQ ID NO: 40, optionally with 1, 2, 3, 4, 5, 6 or 7 modification(s) selected from substitution(s), addition(s), deletion(s) and any combination thereof, preferably amino acid substitutions;
a LCDR1 of SEQ ID NO: 33, optionally with 1 amino acid modification selected from substitution, addition, deletion, preferably an amino acid substitution;
a LFR2 of SEQ ID NO: 41, optionally with 1, 2 or 3 modification(s) selected from substitution(s), addition(s), deletion(s) and any combination thereof, preferably amino acid substitutions;
a LCDR2 of SEQ ID NO: 34, optionally with 1 amino acid modification selected from substitution, addition, deletion, preferably an amino acid substitution;
a LFR3 of SEQ ID NO: 42, optionally with 1, 2, 3, 4, 5, 6, 7, or 8 modification(s) selected from substitution(s), addition(s), deletion(s) and any combination thereof, preferably amino acid substitutions;
a LCDR3 of SEQ ID NO: 35; and
a LFR4 of SEQ ID NO: 43, optionally with 1 amino acid modification selected from substitution, addition, deletion, preferably an amino acid substitution.

In another aspect, the anti-CD28 antigen-binding domain comprises or consists essentially of:
(a) a heavy chain variable region (VH) comprising or consisting of an amino acid sequence selected from the group consisting of SEQ ID NO: 44, 83, 84, 85 and 88, optionally with one, two or three amino acid modification(s) selected from substitution(s), addition(s), deletion(s) and any combination thereof, preferably substitution(s); and (b) a light chain variable region (VL) comprising or consisting of an amino acid sequence selected from the group consisting of SEQ ID NO: 45, 86, 87, 89 and 90, optionally with one, two or three amino acid modification(s) selected from substitution(s), addition(s), deletion(s) and any combination thereof, preferably substitution(s).

Preferably, the one, two or three modification(s) in the VH and/or VL are outside of the CDRs (i.e., are in the framework regions).

In another aspect, the anti-CD28 antigen-binding domain comprises or consists essentially of:
(a) a heavy chain variable region (VH) comprising or consisting of an amino acid sequence selected from the group consisting of SEQ ID NO: 44, 83, 84, 85 and 88; and (b) a light chain variable region (VL) comprising or consisting of an amino acid sequence of SEQ ID NO: 45, optionally with 1, 2 or 3 modification(s) selected from substitution(s), addition(s), deletion(s) and any combination thereof, preferably amino acid substitutions.

In another aspect, the anti-CD28 antigen-binding domain comprises or consists essentially of:
(a) a heavy chain variable region (VH) comprising or consisting of an amino acid sequence of SEQ ID NO: 44 optionally with 1, 2 or 3 modification(s) selected from substitution(s), addition(s), deletion(s) and any combination thereof, preferably amino acid substitutions; and (b) a light chain variable region (VL) comprising or consisting of an amino acid sequence selected from the group consisting of SEQ ID NO: 45, 86, 87, 89 and 90.

In another aspect, the anti-CD28 antigen-binding domain comprises or consists essentially of:
(a) a heavy chain variable region (VH) comprising or consisting of an amino acid sequence as set forth in SEQ ID NO: 44 or a sequence having 80%, 85%, 90%, 95%, 97% or 99% sequence identity thereto; and (b) a light chain variable region (VL) comprising or consisting of an amino acid sequence as set forth in SEQ ID NO: 45 or a sequence having 80%, 85%, 90%, 95%, 97% or 99% sequence identity thereto.

### Fc Domain

The Fc domain of the multifunctional molecule can form together with a part of the antigen binding domain a heavy chain of an IgG immunoglobulin. Indeed, the multifunctional molecule may particularly comprise one heavy chain, including the variable heavy chain (VH), CH1, hinge, CH2 and CH3 domains. However, the multifunctional molecule may also comprise other antigen binding domain structures such as scFv covalently linked to a Fc domain.

The Fc domain can be from a heavy chain constant domain of a human immunoglobulin heavy chain, for example, IgG1, IgG2, IgG3, IgG4, or other classes. Preferably, the multifunctional molecule comprises an IgG1 or an IgG4 heavy chain constant domain.

The multifunctional molecule may particularly comprises a CH1 domain, a CH2 domain and a CH3 domain. Suitable heavy chain constant domains (CH1, CH2 and CH3) are for example as provided in amino acid sequence SEQ ID NO 23 and 24.

The multifunctional molecule preferably comprises a Fc domain. Such Fc domain preferably comprises a first Fc chain and a second Fc chain that are complementary and are able to dimerize. In certain embodiments, the first Fc chain comprises an amino acid sequence that differs from that of the second Fc chain in one or more amino acid addition, deletion or substitution. Such Fc first and second Fc chain form a “heterodimeric Fc”. In other embodiments, the first and second Fc chains comprise the same amino acid sequence and form a “homodimeric Fc”.

Preferably, the Fc domain comprises CH2 and CH3 domains. Optionally, it can include all or a portion of the hinge region, the CH2 domain and/or the CH3 domain. In some aspects, the CH2 and/or a CH3 domains are derived from a human IgG4 or IgG1 heavy chain. Preferably, the Fc domain includes all or a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4. More preferably, the hinge region is derived from a human or humanized IgG1 or IgG4 heavy chain.

The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some aspects, the first cysteine within the human IgG1 hinge region is mutated to another amino acid, preferably serine.

The hinge region of IgG4 may form interchain disulfide bonds inefficiently. Suitable hinge region for the present invention can be derived from the IgG4 hinge region, preferably containing a mutation that enhances correct formation of disulfide bonds between heavy chain-derived moieties (for example such as described in Angal S, et al. (1993) Mol. Immunol., 30:105-8). More preferably, the hinge region is derived from a human IgG4 heavy chain.

In an embodiment, the multifunctional molecule comprises a hinge region, in particular comprising or consisting of the amino acid sequence as set forth in SEQ ID NO: 58, optionally with one, two or three modification(s) selected from substitution(s), addition(s), deletion(s) and any combination thereof.

The multifunctional molecule preferably comprises a dimeric Fc domain. Accordingly, the multifunctional molecule comprises two Fc domains, the Fc chains being able to form a dimeric Fc domain. The dimeric Fc domain can be a homodimer, each Fc monomer (or chain) being identical or essentially identical. Alternatively, the dimeric Fc domain can be a heterodimer, each Fc monomer (or chain) being different and complementary in order to promote the formation of the heterodimeric Fc domain.

More specifically, the Fc domain is a heterodimeric Fc domain. Heterodimeric Fc domains can be made by altering the amino acid sequence of each monomer (i.e., each of the Fc chain forming the Fc domain). The heterodimeric Fc domains rely on amino acid variants in the constant regions that are different on each chain to promote heterodimeric formation and/or allow for ease of purification of heterodimers over the homodimers. There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. Heterodimerization variants can include steric variants (e.g., the “knobs and holes” or “skew” variants described below and the “charge pairs” variants described below) as well as “pi variants”, which allows purification of homodimers away from heterodimers. WO2014/145806, hereby incorporated by reference in its entirety, discloses useful mechanisms for heterodimerization include “knobs and holes”, “electrostatic steering” or “charge pairs”, pi variants, and general additional Fc variants. See also, Ridgway et al., Protein Engineering 9(7):617 (1996); Atwell et al., J. Mol. Biol. 1997 270:26; U.S. Pat. No. 8,216,805, Merchant et al., Nature Biotech. 16:677 (1998), all of which are hereby incorporated by reference in their entirety. For “electrostatic steering” see Gunasekaran et al., J. Biol. Chem. 285(25): 19637 (2010), hereby incorporated by reference in its entirety. For pi variants, see US 2012/0149876 hereby incorporated by reference in its entirety.

Then, in a preferred aspect, the heterodimeric Fc domain comprises a first Fc chain and a complementary second Fc chain based on the “knobs and holes” technology. For instance, the first Fc chain is a “knob” or K chain, meaning that it comprises the substitution characterizing a knob chain, and the second Fc chain is a “hole” or H chain, meaning that it comprises the substitution characterizing a hole chain. And vice versa, the first Fc chain is a “hole” or H chain, meaning that it comprises the substitution characterizing a hole chain, and the second Fc chain is a “knob” or K chain, meaning that it comprises the substitution characterizing a knob chain.

In a preferred aspect, the Fc chain that is linked to the anti-CD28 binding domain (i.e., the second Fc chain) is a “hole” or H chain. Alternatively, the Fc chain that is linked to the anti-CD28 binding domain (i.e., the second Fc chain) is a “knob” or K chain.

Optionally, the heterodimeric Fc domain may comprise one heterodimeric Fc chain which comprises the substitutions as shown in the following table E and the other heterodimeric Fc chain comprising the substitutions as shown in the following table E.

TABLE E
(the numbering being according to EU index).

| Fc chain having the | The complementary Fc chain |
|---|---|
| following substitutions | having the following substitutions |
| (Hole chain or H chain) | (Knob chain or K chain) |
| D221E/P228E/L368E | D221R/P228R/K409R |
| C220E/P228E/368E | C220R/E224R/P228R/K409R |
| S364K/E357Q | L368D/K370S |
| L368D/K370S | S364K |
| L368E/K370S | S364K |
| T411T/E360E/Q362E | D401K |
| L368D/K370S | S364K/E357L |
| K370S | S364K/E357Q |
| T366S/L368A/Y407V | T366W |
| T366S/L368A/Y407V/Y349C | T366W/S354C |
| F368D/K370S | S364K |
| F368D/K370S | S364K/E357F |
| F368D/K370S | S364K/E357Q |
| T411E/K360E/Q362E | D401K |
| F368E/K370S | S364K |
| K370S | S364K/E357Q |
| T366S/F368A/Y407V | T366W |
| T366S/L368A/Y407V/Y349C | T366W/S354C |

In a preferred aspect, the first Fc chain is a “hole” or H chain and comprises the substitutions T366S/L368A/Y407V/Y349C and the second Fc chain is a “knob” or K chain and comprises the substitutions T366W/S354C.

Optionally, the Fc chain may further comprise additional substitutions.

In particular, for multifunctional molecules that target cell-surface molecules, especially those on immune cells, abrogating effector functions may be required. Engineering Fc regions may also be desired to either reduce or increase the effector function of the multifunctional molecules.

In certain aspects, amino acid modifications may be introduced into the Fc region to generate an Fc region variant. In certain aspects, the Fc region variant possesses some, but not all, effector functions. Such multifunctional molecules may be useful, for example, in applications in which the half-life of the antibody in vivo is important, yet certain effector functions are unnecessary or deleterious. Numerous substitutions or substitutions or deletions with altered effector function are known in the art.

In one aspect, the constant region of the Fc domain contains a mutation that reduces affinity for an Fc receptor or reduces Fc effector function. For example, the constant region can contain a mutation that eliminates the glycosylation site within the constant region of an IgG heavy chain. Preferably, the CH2 domain contains a mutation that eliminates the glycosylation site within the CH2 domain.

In a particular aspect, the Fc domain is modified to increase the binding to FcRn, thereby increasing the half-life of the multifunctional molecule. In another aspect or additional aspect, the Fc domain is modified to decrease the binding to FcγR, thereby reducing ADCC or CDC, or to increase the binding to FcγR, thereby increasing ADCC or CDC.

The alteration of amino acids near the junction of the Fc portion and the non-Fc portion can dramatically increase the serum half-life of the Fc fusion protein as shown in WO 01/58957. Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life.

In one embodiment, the constant region of the Fc domain has one of the mutations described in the Table F below, or any combination thereof.

TABLE F
Suitable human engineered Fc domain of an antibody. numbering of residues in the heavy chain
constant region is according to EU numbering (Edelman, G. M. et al., Proc. Natl. Acad. USA, 63,
78-85 (1969); www.imgt.org/IMGTScientificChart/Numbering/Hu_IGHGnber.html#refs)

| Engineered | | | | |
|---|---|---|---|---|
| Fc | Isotype | Mutations | FcR/C1q Binding | Effector Function |
| hIgG1e1-Fc | IgG1 | T250Q/M428L | Increased binding | Increased half-life |
| | | | to FcRn | |
| hIgG1e2-Fc | IgG1 | M252Y/S254T/T256E + | Increased binding | Increased half-life |
| | | H433K/N434F | to FcRn | |
| hIgG1e3-Fc | IgG1 | E233P/L234V/L235A/G236A + | Reduced binding | Reduced ADCC and |
| | | A327G/A330S/P331S | to FcγRI | CDC |
| hIgG1e4-Fc | IgG1 | E333A | Increased binding | Increased ADCC and |
| | | | to FcγRIIIa | CDC |
| hIgG1e5-Fc | IgG1 | S239D/A330L/1332E | Increased binding | Increased ADCC |
| | | | to FcγRIIIa | |
| hIgG1e6-Fc | IgG1 | P2571/Q311 | Increased binding | Unchanged half-life |
| | | | to FcRn | |
| hIgG1e7-Fc | IgG1 | K326W/E333S | Increased binding | Increased CDC |
| | | | to C1q | |
| hIgG1e9-Fc | IgG1 | S239D/1332E/G236A | Increased | Increased |
| | | | FcγRIIa/FcγRIIb | macrophage |
| | | | ratio | phagocytosis |
| hIgG1e9-Fc | IgG1 | N297A | Reduced binding | Reduced ADCC and |
| | | | to FcγRI | CDC |
| hIgG1e9-Fc | IgG1 | LALA (L234A/L235A) | Reduced binding | Reduced ADCC and |
| | | | to FcγRI | CDC |
| hIgG1e10- | IgG1 | N297A + YTE | Reduced binding | Reduced ADCC and |
| Fc | | (N298A + | to FcγRI | CDC |
| | | M252Y/S254T/T256E) | Increased binding | Increased half-life |
| | | | to FcRn | |
| hIgG1e11- | IgG1 | K322A | Reduced binding | Reduced CDC |
| Fc | | | to C1q | |
| hIgG1e12- | IgG1 | N297A + YTE | | Reduced ADCC and |
| Fc | | (N298A + | | CDC |
| | | M252Y/S254T/T256E) + | | Increased half-life |
| | | K444A | | Abolish cleavage of |
| | | | | the C-terminal lysine |
| | | | | of the antibody |
| hIgG4e1-Fc | IgG4 | S228P | — | Reduced Fab-arm |
| | | | | exchange |
| hIgG4e1-Fc | IgG4 | LALA (L234A/L235A) | Increased binding | Increased half-life |
| | | | to FcRn | |
| hIgG4e2-Fc | IgG4 | S228P + YTE (S228P + | — | Reduced Fab-arm |
| | | M252Y/S254T/T256E) | Increased binding | exchange |
| | | | to FcRn | Increased half-life |
| hIgG4e3-Fc | IgG4 | N297A + YTE | | Reduced ADCC and |
| | | (N298A + | | CDC |
| | | M252Y/S254T/T256E) + | | Increased half-life |
| | | K444A | | Abolish cleavage of |
| | | | | the C-terminal lysine |
| | | | | of the antibody |

In a particular aspect, the multifunctional molecule comprises a human IgG1 heavy chain constant domain or an IgG1 Fc domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/1332E; P2571/Q311; K326W/E333S; S239D/1332E/G236A; N297A; L234A/L235A; P329G; N297A+M252Y/S254T/T256E; K322A, K444A, K444E, K444D, K444G, K444S, M428L, L309D, Q311H, N434S, M428L+N434S and L309D+Q311H+N434S, preferably selected from the group consisting of N297A optionally in combination with M252Y/S254T/T256E, and L234A/L235A optionally with P329G.

In another aspect, the multifunctional molecule comprises a human IgG4 heavy chain constant domain or a human IgG4 Fc domain, optionally with a substitution or a combination of substitutions selected from the group consisting of S228P; L234A/L235A; L234A/L235A/P329G, P329G, S228P+M252Y/S254T/T256E, K444A K444E, K444D, K444G and K444S. Even more preferably, the multifunctional molecule, preferably the multifunctional molecule according to the invention comprises an IgG4 Fc-region with a S228P that stabilizes the IgG4.

As mentioned herein the “/” and “+” refer to mutations that are cumulative. Thus, by the mutation S228P+M252Y/S254T/T256E, it is meant the following mutations: S228P, M252Y, S254T and T256E.

Generally, all subclass of Human IgG carries a C-terminal lysine residue of the antibody heavy chain (K444) that are susceptible to be cleaved off in circulation. This cleavage in the blood may compromise or decrease the bioactivity of the multifunctional molecule by releasing the linked immune-stimulating moiety to the multifunctional molecule. To circumvent this issue, K444 amino acid in the IgG domain can be substituted by another amino acid to reduce proteolytic cleavage, a mutation commonly used for antibodies. Then, in one aspect, the multifunctional molecule comprises at least one further amino acid substitution consisting of K444A, K444E, K444D, K444G or K444S, preferentially K444A. Particularly, K444 amino acid in the IgG domain can be substituted by an alanine to reduce proteolytic cleavage, a mutation commonly used for antibodies. Then, in one aspect, the multifunctional molecule comprises at least one further amino acid substitution consisting of K444A.

Optionally, the multifunctional molecule comprises an additional cysteine residue at the C-terminal domain of the Fc domain to create an additional disulfide bond and potentially restrict the flexibility of the multifunctional molecule.

In one particular aspect, the multifunctional molecule according to the invention comprises a heterodimer of Fc domains that comprises the “knob into holes” modifications such as described above. Preferably, such Fc domains are IgG1 or IgG4 Fc domain such as described above, even more preferably an IgG1 Fc domain comprising the mutation N297A such as disclosed above.

In some embodiments, the Fc chain linked to the first binding moiety, in particular to the anti-PD1 binding domain chain may comprise or consist of the amino acid sequence as set forth in SEQ ID NO: 25 or 26, optionally with one, two or three modification(s) selected from substitution(s), addition(s), deletion(s) and any combination thereof.

In some embodiments, the Fc chain linked to the anti-CD28 bidding domain is a “hole” or H chain and comprises the substitutions T366S/L368A/Y407V/Y349C and optionally N297A and the other Fc chain is a “knob” or K chain and comprises the substitutions T366W/S354C and optionally N297A. Preferably, he Fc chain linked to the anti-CD28 bidding domain is a “hole” or H chain and comprises or consists of the amino acid sequence as set forth in SEQ ID NO: 54.

More particularly, the multifunctional molecule may comprise a Fc domain comprising or consisting of:
i) a first Fc chain comprising or consisting of the amino acid sequence as set forth in SEQ ID NO: 25 (knob) optionally with one, two or three modification(s) selected from substitution(s), addition(s), deletion(s) and any combination thereof.
ii) a second Fc chain comprising or consisting of the amino acid sequence as set forth in SEQ ID NO: 26 or 54 (hole) optionally with one, two or three modification(s) selected from substitution(s), addition(s), deletion(s) and any combination thereof.

In a first embodiment, the multifunctional molecule comprises an anti-CD28 binding domain linked to the C-terminal end of the Fc domain, said Fc domain being a hole chain.

In a second embodiment, the multifunctional molecule comprises an anti-CD28 binding domain linked to the N-terminal end of the Fc domain, said Fc domain being a hole chain.

In a third embodiment, the multifunctional molecule comprises an anti-CD28 binding domain linked to the C-terminal end of the Fc domain, said Fc domain being a knob chain.

In a fourth embodiment, the multifunctional molecule comprises an anti-CD28 binding domain linked to the N-terminal end of the Fc domain, said Fc domain being a knob chain.

### Peptide Linker

This invention includes a multifunctional molecule which may comprise one or more peptide linker. The peptide linker usually has a length and flexibility enough to ensure that the two protein elements connected with the linker in between have enough freedom in space to exert their functions and avoid influences of the formation of a-helix and β-fold on the stability of the recombinant multifunctional molecule.

As used herein, the term “peptide linker” refers to a sequence of at least one amino acid that links different domain or chain of the multifunctional molecule. Such a linker may be useful to prevent steric hindrances. The linker is usually 3-44 amino acid residues in length. Preferably, the linker has 3-30 amino acid residues. In some embodiments, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker sequence may be a naturally occurring sequence or a non-naturally occurring sequence. If used for therapeutic purposes, the linker is preferably non-immunogenic in the subject to which the multifunctional molecule is administered. One useful group of linker sequences are linkers derived from the hinge region of heavy chain antibodies as described in WO 96/34103 and WO 94/04678.

Other examples are poly-alanine linker sequences. Further preferred examples of linker sequences are Gly/Ser linkers of different length including (Gly4Ser)₄, (Gly4Ser)₃, (Gly4Ser)₂, Gly4Ser, Gly3Ser, Gly3, Gly2ser and (Gly3Ser2)₃, in particular (Gly4Ser)₃.

In one embodiment, the one or more linker comprised in the multifunctional molecule is selected in the group consisting of (Gly4Ser)₄, (Gly4Ser)₃, (Gly4Ser)₂, Gly4Ser, Gly3Ser, Gly3, Gly2ser and (Gly3Ser2)₃, preferably is (Gly4Ser)₃.

In one embodiment, the one or more linker comprised in the multifunctional molecule is selected in the group consisting of (GGGGS)₄, (GGGGS)₃, (GGGGS)₂, GGGGS, (GGGS)₃, (GGGS)₂, GGGS, GGG, GGS and GG, and is preferably (GGGGS)₃.

In an embodiment, the anti-CD28 binding moiety is a scFv and it is covalently linked to a Fc domain by a peptide linker such as described above, in particular a (GGGGS)₃ linker, in particular to the N or C terminal end of a Fc domain such as described herein.

In an embodiment, the C terminus of the VL domain of the anti-CD28 antigen binding domain can be linked to the N terminus of a Fc domain by a peptide linker such as described above, in particular a (GGGGS)₃ linker.

Alternatively, the C terminus of the VH domain of the anti-CD28 antigen binding domain is linked to the N terminus of a Fc domain by a peptide linker such as described above, in particular a (GGGGS)₃ linker.

In an embodiment, the N terminus of the VL domain of the anti-CD28 antigen binding domain can be linked to the C terminus of a Fc domain by a peptide linker such as described above, in particular a (GGGGS)₃ linker.

Alternatively, the N terminus of the VH domain of the anti-CD28 antigen binding domain is linked to the C terminus of a Fc domain by a peptide linker such as described above, in particular a (GGGGS)₃ linker.

In an embodiment, the anti-CD28 binding moiety is a scFv and is covalently linked to a CL domain of the first binding moiety by a peptide linker such as described above, in particular a (GGGGS)₃ linker.

In an embodiment, the C terminus of the VL of the anti-CD28 antigen binding domain can be linked to the N terminus of a CL domain of the first binding moiety by a peptide linker such as described above, in particular a (GGGGS)₃ linker, such as described in SEQ ID NO: 57.

Alternatively, the C terminus of the VH of the anti-CD28 antigen binding domain is linked to the N terminus of a CL domain of the first binding moiety by a peptide linker such as described above, in particular a (GGGGS)₃ linker, such as described in SEQ ID NO: 57.

In an embodiment, the N terminus of the VL of the anti-CD28 antigen binding domain can be linked to the C terminus of a CL domain of the first binding moiety by a peptide linker such as described above, in particular a (GGGGS)₃ linker, such as described in SEQ ID NO: 57.

Alternatively, the N terminus of the VH of the anti-CD28 antigen binding domain is linked to the C terminus of a CL domain of the first binding moiety by a peptide linker such as described above, in particular a (GGGGS)₃ linker, such as described in SEQ ID NO: 57.

In an embodiment, wherein the anti-CD28 antigen binding domain is an scFv, and the VL and VH domains of said scFv are preferably covalently linked to each other by a peptide linker such as described above, in particular a (GGGGS)₃ linker, such as described in SEQ ID NO: 57.

Particularly, the C terminus of the VL of the anti-CD28 antigen binding domain is linked to the N terminus of the VH of the anti-CD28 antigen binding domain by a peptide linker such as described above, in particular a (GGGGS)₃ linker, such as described in SEQ ID NO: 57.

Alternatively, the C terminus of the VH of the anti-CD28 antigen binding domain is linked to the N terminus of the VL of the anti-CD28 antigen binding domain by a peptide linker such as described above, in particular a (GGGGS)₃ linker, such as described in SEQ ID NO: 57.

Thus, in some aspects, the anti-CD28 binding domain is a scFV comprising from the N-terminus to the C-terminus: a VH domain of SEQ ID NO: 44, a peptide linker of SEQ ID NO: 57, and a VL domain of SEQ ID NO: 45. Alternatively, the anti-CD28 binding domain is a scFV comprising from the N-terminus to the C-terminus: a VL domain of SEQ ID NO: 45, a peptide linker of SEQ ID NO: 57, and a VH domain of SEQ ID NO: 44.

At the linkage junction, the C-terminal lysine residue of the VL or VH can be mutated to alanine to reduce proteolytic cleavage.

Accordingly, the multifunctional molecule comprises a first binding moiety binding to a target specifically expressed on T cells surface linked by its C-terminal end to N-terminal end of a first Fc chain, an anti-CD28 binding domain being covalently linked, optionally through a peptide linker, by its C-terminal end to N-terminal end of a second Fc chain, wherein the first and second Fc chains are complementary. Preferably, the anti-CD28 binding domain is a scFv so that the molecule may also comprise a peptide linker between the VL and VH domain of said scFv.

In another embodiment, the multifunctional molecule comprises a first binding moiety binding to a target specifically expressed on T cells surface linked by its C-terminal end to N-terminal end of a first Fc chain, an anti-CD28 binding domain being covalently linked, optionally through a peptide linker, by its N-terminal end to the C-terminal end of said first Fc chain, a complementary second Fc chain is devoid of binding moiety. Preferably, the anti-CD28 binding domain is a scFv so that the molecule may also comprise a peptide linker between the VL and VH domain of said scFv.

In another embodiment, the multifunctional molecule comprises a Fc domain linked to two first binding moiety binding to a target specifically expressed on T cells surface comprising VH and VL domains, and an anti-CD28 binding domain covalently linked, optionally through a peptide linker, by its C-terminal end to the N-terminal end of the light chain of the first binding moiety or by its N-terminal end to the C-terminal end of the Fc domain or to the C-terminal end of light chain of the first binding moiety. Preferably, the anti-CD28 binding domain is a scFv so that the molecule also comprises a peptide linker between the VL and VH domain of said scFv.

### Examples of Multifunctional Molecules

Here is provided particular examples of multifunctional molecules according to the invention, wherein the multifunctional molecule comprises or consists of:
a first binding moiety that is an anti-PD1 binding moiety, in particular an anti-PD1 binding domain selected from the group consisting of a Fab, a Fab′, a F(ab′)2, a CrossMAb, or a single chain (scFv), said anti-PD1 binding moiety having preferably the amino-acid sequence described above under the section “first binding moiety”;
a second binding moiety that is an anti-CD28 binding moiety with an agonistic activity, in particular an antigen binding domain selected from the group consisting of a Fab, a Fab′, a CrossMAb or a single chain (scFv), said anti-CD28 binding moiety having preferably the amino-acid sequence described above under the section “second binding moiety”; and
a first and a second Fc chains, said first and second Fc chains being complementary and forming together a Fc domain;
and wherein said multifunctional molecule comprises a single anti-CD28 binding.

Particular examples of multifunctional molecules are provided in FIG. 1, Format A1, B1, B2, B3, B4, C1, C2, C3, C4, C5 and C6.

Preferably, the bifunctional molecule has a B1 format, in particular such as described in FIG. 7A. Particularly, the B1 format consists essentially of i) an anti-PD-1 Fab linked to a first Fc chain and ii) an anti-CD28 scFv linked to a second Fc chain; the first and second Fc chain being complementary so as to form a Fc domain.

In other words, the multifunctional molecule binds CD28 monovalently and is unable to crosslink CD28. In all and any of the specific examples of multifunctional molecules disclosed in this section and comprising an anti-PD1 binding moiety as a first binding moiety, the anti-PD1 binding moiety can be replaced by another binding moiety binding to another target specifically expressed on T cells surface, in particular such as CTLA-4, BTLA, VISTA, TIGIT, CD160, LAG3 and TIM3.

In a first aspect, the multifunctional molecule comprises or consists of:
a first binding moiety that is an anti-PD1 Fab linked to a first Fc chain,
a second binding moiety that is an anti-CD28 agonist Fab or CrossMAb linked to a second Fc chain; and
said first and second Fc chains being complementary and forming together a Fc domain;
and said multifunctional molecule comprises a single anti-CD28 binding moiety.

More specifically, said multifunctional molecule comprises or consists of:
a first chain comprising or consisting of, from N-terminus to C-terminus, VL and CL domains of the anti-PD1 binding moiety,
a second chain comprising or consisting of, from N-terminus to C-terminus, VH and CH1 domains of the anti-PD1 binding moiety, optionally a hinge, CH2 and CH3 domains of an antibody, said CH2 and CH3 domains forming a first Fc chain,
a third chain comprising or consisting of, from N-terminus to C-terminus, (i) (Fab) VH and CH1 domains of the anti-CD28 binding moiety, optionally a hinge, CH2 and CH3 domains of an antibody, the CH2 and CH3 domains forming together the second Fc chain; or (ii) (CrossMAb) VH and CL domains of the anti-CD28 binding moiety, optionally a hinge, CH2 and CH3 domains of an antibody, the CH2 and CH3 domains forming together a second Fc chain,
a fourth chain comprising or consisting of, from N-terminus to C-terminus, (i) (Fab) VL and CL domains of the anti-CD28 binding moiety; or (ii) (CrossMAb) VL and CH1 domains of the anti-CD28 binding moiety,
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably, the multifunctional molecule comprises or consists of:
a first chain that is a light chain of an anti-PD1 antibody and comprising or consisting of, from N-terminus to C-terminus, VL and CL domains comprising or consisting of SEQ ID NOs: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that is a heavy chain of an anti-CD28 antibody and comprising or consisting of, from N-terminus to C-terminus, (i) VH and CH1 domains comprising or consisting of SEQ ID NOs: 44 and 46, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the second Fc chain comprising or consisting of SEQ ID NO: 54, optionally with one, two or three modification(s) selected from substitution(s), addition(s), deletion(s) and any combination thereof;
a fourth chain that is a light chain of an anti-CD28 antibody and comprising or consisting of, from N-terminus to C-terminus, VL and CL domains of an anti-CD28 antibody comprising or consisting of SEQ ID NO: 45 and 47, respectively, optionally with one, two or three modification(s) selected from substitution(s), addition(s), deletion(s) and any combination thereof,
   wherein the first and second Fc chains are complementary and thereby form a Fc domain.

Alternatively the multifunctional molecule comprises or consists of:
a first chain that is a light chain of an anti-PD1 antibody and comprising or consisting of, from N-terminus to C-terminus, VL and CL domains comprising or consisting of SEQ ID NOs:16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that is a heavy chain of an anti-CD28 antibody and comprising or consisting of, from N-terminus to C-terminus, (i) VH and CL domains comprising or consisting of SEQ ID NOs: 44 and 47, respectively, optionally with one, two or three modification(s) selected from substitution(s), addition(s), deletion(s) and any combination thereof; ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the second Fc chain comprising or consisting of SEQ ID NO: 54, optionally with one, two or three modification(s) selected from substitution(s), addition(s), deletion(s) and any combination thereof;
a fourth chain that is a light chain of an anti-CD28 antibody and comprising or consisting of, from N-terminus to C-terminus, VL and CH1 domains of an anti-CD28 antibody comprising or consisting of SEQ ID NOs: 45 and 46, respectively, optionally with one, two or three modification(s) selected from substitution(s), addition(s), deletion(s) and any combination thereof,
   wherein the first and second Fc chains are complementary and thereby form a Fc domain.

In a very particular embodiment, the multifunctional molecule comprises or consists of:
a first chain that comprises or consists of the sequence as set forth in SEQ ID NO: 20, optionally with one, two or three modification(s) selected from substitution(s), addition(s), deletion(s) and any combination thereof;
a second chain that comprises or consists of the sequence as set forth in SEQ ID NO: 27, optionally with one, two or three modification(s) selected from substitution(s), addition(s), deletion(s) and any combination thereof;
a third chain that comprises or consists of the sequence as set forth in SEQ ID NO: 53, optionally with one, two or three modification(s) selected from substitution(s), addition(s), deletion(s) and any combination thereof, and
a fourth chain that comprises or consists of the sequence as set forth in SEQ ID NO: 51, optionally with one, two or three modification(s) selected from substitution(s), addition(s), deletion(s) and any combination thereof.

In a second aspect, the multifunctional molecule comprises or consists of:
a first binding moiety that is an anti-PD1 Fab, Fab′ or CrossMAb;
a second binding moiety that is an agonistic anti-CD28 scFV; and
a first Fc chain and a second Fc chain, said first and second Fc chains being complementary and forming together a Fc domain.

In one embodiment, said multifunctional molecule particularly comprises or consists of:
a first chain comprising or consisting of, from N-terminus to C-terminus, VL and CL domains of an anti-PD1 antibody,
a second chain comprising or consisting of, from N-terminus to C-terminus, VH and CH1 domains, optionally a hinge, CH2 and CH3 domains of an antibody, said CH2 and CH3 domains forming together the first Fc chain,
a third chain comprising or consisting of, from N-terminus to C-terminus, VL and VH domains of an anti-CD28 antibody linked by a peptide linker, optionally a peptide linker, optionally a hinge, CH2 and CH3 domains of an antibody, said CH2 and CH3 domains forming together the second Fc chain,
wherein the first and second Fc chains are complementary and thereby form together a Fc domain.

In particular, the first Fc chain is a “knob” and the second Fc chain is a “hole”. Alternatively, the first Fc chain is a “hole” and the second Fc chain is a “knob”.

Preferably, the multifunctional molecule comprises or consists of:
a first chain that is a light chain of an anti-PD1 antibody and comprising or consisting of, from N-terminus to C-terminus, VL and CL domains comprising or consisting of SEQ ID NOs: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and a CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains, said CH2 and CH3 domains forming the first Fc chain comprising or consisting of SEQ ID NO: 25, optionally with one, two or three modification(s) selected from substitution(s), addition(s), deletion(s) and any combination thereof; and
a third chain comprising or consisting of (i) an anti-CD28 scFv comprising VL and VH domains of an anti-CD28 antibody comprising or consisting of SEQ ID NOs: 45 and 44, respectively, optionally with one, two or three modification(s) selected from substitution(s), addition(s), deletion(s) and any combination thereof, preferably linked by a peptide linker, preferably of SEQ ID NO: 57 (ii) optionally a peptide linker, (iii) optionally a hinge, preferably of SEQ ID NO: 58, optionally with one, two or three modification(s) selected from substitution(s), addition(s), deletion(s) and any combination thereof iv) CH2 and CH3 domains of an antibody, the CH2 and CH3 domains forming the second Fc chain comprising or consisting of SEQ ID NO: 26 or 54, preferably of SEQ ID NO: 54 optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 20, optionally with one, two or three modification(s) selected from substitution(s), addition(s), deletion(s) and any combination thereof
a second chain that comprises or consists of the sequence as set forth in SEQ ID NO: 27, optionally with one, two or three modification(s) selected from substitution(s), addition(s), deletion(s) and any combination thereof, and
a third chain that comprises or consists of the sequence as set forth in SEQ ID NO: 56 optionally with one, two or three modification(s) selected from substitution(s), addition(s), deletion(s) and any combination thereof.

In a preferred embodiment, the multifunctional molecule of the invention comprises or consists of:
a first chain that comprises or consists of the sequence as set forth in SEQ ID NO: 20, optionally with one, two or three modification(s) selected from substitution(s), addition(s), deletion(s) and any combination thereof; preferably amino acid substitution(s); said modifications being preferably in the framework regions;
a second chain that comprises or consists of the sequence as set forth in SEQ ID NO: 27, optionally with one, two or three modification(s) selected from substitution(s), addition(s), deletion(s) and any combination thereof, preferably amino acid substitution(s); said modifications being preferably in the framework regions; and
a third chain that comprises or consists of the sequence as set forth in SEQ ID NO: 56 optionally with one, two or three modification(s) selected from substitution(s), addition(s), deletion(s) and any combination thereof; preferably amino acid substitution(s); said modifications being preferably in the framework regions.

In one embodiment, the multifunctional molecule particularly comprises or consists of:
a first chain comprising or consisting of, from N-terminus to C-terminus, VL and CL domains of an anti-PD1 antibody;
a second chain comprising or consisting of, from N-terminus to C-terminus, VH and CH1 domains of an anti-PD1 antibody, optionally a hinge, CH2 and CH3 domains of an antibody, said CH2 and CH3 domains forming the first Fc chain; and
a third chain comprising or consisting of, from N-terminus to C-terminus, VH and VL domains of an anti-CD28 antibody, preferably linked by a peptide linker, optionally a peptide linker, optionally a hinge, CH2 and CH3 domains of an antibody, said CH2 and CH3 domains forming the second Fc chain;
wherein the first and second Fc chains are complementary and thereby form together a Fc domain.

In particular, the first Fc chain is a “knob” and the second Fc chain is a “hole”. Alternatively, the first Fc chain is a “hole” and the second Fc chain is a “knob”.

Preferably the multifunctional molecule comprises or consists of:
a first chain that is a light chain of an anti-PD1 antibody and comprising or consisting of, from N-terminus to C-terminus, VL and CL domains comprising or consisting of SEQ ID NOs: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof; and
a third chain comprising or consisting of, from N-terminus to C-terminus, (i) an anti-CD28 scFv comprising VH and VL domains of an anti-CD28 antibody comprising or consisting of SEQ ID NOs: 44 and 45, respectively, optionally with one, two or three modification(s) selected from substitution(s), addition(s), deletion(s) and any combination thereof, preferably linked by a peptide linker, preferably of SEQ ID NO: 57 (ii) optionally a peptide linker, preferably of SEQ ID NO: 57, (iii) optionally a hinge, preferably of SEQ ID NO: 58, optionally with one, two or three modification(s) selected from substitution(s), addition(s), deletion(s) and any combination thereof iv) CH2 and CH3 domains of an antibody, the CH2 and CH3 domains forming the second Fc chain comprising or consisting of SEQ ID NO: 26 or 54, preferably of SEQ ID NO: 54 optionally with one, two or three modification(s) selected from substitution(s), addition(s), deletion(s) and any combination thereof;
wherein the first and second Fc chains are complementary and thereby form a Fc domain.

In a very particular embodiment, the multifunctional molecule comprises or consists of:
a first chain that comprises or consists of the sequence as set forth in SEQ ID NO: 20, optionally with one, two or three modification(s) selected from substitution(s), addition(s), deletion(s) and any combination thereof
a second chain that comprises or consists of the sequence as set forth in SEQ ID NO: 27, optionally with one, two or three modification(s) selected from substitution(s), addition(s), deletion(s) and any combination thereof, and
a third chain that comprises or consists of the sequence as set forth in SEQ ID NO: 55, optionally with one, two or three modification(s) selected from substitution(s), addition(s), deletion(s) and any combination thereof.

Preferably, the one, two or three modification(s) are preferably outside of the CDRs (i.e., are in the framework regions).

In one embodiment the multifunctional molecule particularly comprises or consists of:
a first chain comprising or consisting of, from N-terminus to C-terminus, i) optionally a hinge, ii) CH2 and CH3 domains of an antibody, said CH2 and CH3 domains forming the first Fc chain,
a second chain comprising or consisting of, from N-terminus to C-terminus, (i) VH and CH1 domains of anti-PD1 antibody, optionally a hinge, CH2 and CH3 domains of an antibody, said CH2 and CH3 domains forming the second Fc chain, (ii) optionally a peptide linker, and (iii) an anti-CD28 scFV comprising VL and VH domains of an anti-CD28 antibody, preferably linked by a peptide linker; and
a third chain comprising or consisting of, from N-terminus to C-terminus, VL and a CL domains of an anti-PD1 antibody,
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably said multifunctional molecule comprises or consists of:
a first chain that is a Fc chain of an antibody and comprising or consisting of, from N-terminus to C-terminus, i) optionally a hinge, preferably of SEQ ID NO: 58, optionally with one, two or three modification(s) selected from substitution(s), addition(s), deletion(s) and any combination thereof ii) CH2 and a CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof;
a second chain comprising or consisting of, from N-terminus to C-terminus, a) a heavy chain of an anti-PD1 antibody comprising (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains, the CH2 and CH3 domains forming the second Fc chain comprising or consisting of SEQ ID NO: 26, optionally with one, two or three modification(s) selected from substitution(s), addition(s), deletion(s) and any combination thereof; (b) optionally a peptide linker, preferably of SEQ ID NO: 57 (c) an anti-CD28 scFv comprising VL and VH domains of an anti-CD28 antibody comprising or consisting of SEQ ID NOs: 45 and 44, respectively, optionally with one, two or three modification(s) selected from substitution(s), addition(s), deletion(s) and any combination thereof, preferably linked by a peptide linker, preferably of SEQ ID NO: 57 and
a third chain that is a light chain of an anti-PD1 antibody and comprising or consisting of, from N-terminus to C-terminus, VL and CL domains comprising or consisting of SEQ ID NOs: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 25, optionally with one, two or three modification(s) selected from substitution(s), addition(s), deletion(s) and any combination thereof
a second chain that comprises or consists of the sequence as set forth in SEQ ID NO: 62, optionally with one, two or three modification(s) selected from substitution(s), addition(s), deletion(s) and any combination thereof, and
a third chain that comprises or consists of the sequence as set forth in SEQ ID NO: 20 optionally with one, two or three modification(s) selected from substitution(s), addition(s), deletion(s) and any combination thereof.

In such aspect, the first Fc chain, in particular the knob chain, is preferably devoid of any other binding moiety or immunotherapeutic agent.

In one embodiment, the multifunctional molecule particularly comprises or consists of:
a first chain comprising or consisting of, from N-terminus to C-terminus, i) optionally a hinge, ii) CH2 and CH3 domains of an antibody, said CH2 and CH3 domains forming the first Fc chain,
a second chain comprising or consisting of, from N-terminus to C-terminus, (i) VH and CH1 domains of an anti-PD1 antibody, a hinge, CH2 and CH3 domains of an anti-PD1 antibody, said CH2 and CH3 domains forming the second Fc chain, optionally a peptide linker, and (ii) an anti-CD28 scFv comprising VH and VL domains of an anti-CD28 antibody, preferably linked by a peptide linker; and
a third chain comprising or consisting of, from N-terminus to C-terminus, VL and CL domains of an anti-PD1 antibody,
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

In such aspect, the first Fc chain, in particular the knob chain, is preferably devoid of any other binding moiety or immunotherapeutic agent.

Preferably, the multifunctional molecule comprises or consists of:
a first chain that is a first Fc chain of an antibody and comprising or consisting of: i) optionally a hinge, preferably of SEQ ID NO: 58, optionally with one, two or three modification(s) selected from substitution(s), addition(s), deletion(s) and any combination thereof ii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof; said first Fc chain being devoid of any other binding moiety or immunotherapeutic agent;
a second chain comprising or consisting of a) a heavy chain of an anti-PD1 antibody comprising (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the second Fc chain comprising or consisting of SEQ ID NO: 26, optionally with one, two or three modification(s) selected from substitution(s), addition(s), deletion(s) and any combination thereof; (b) optionally a peptide linker, preferably of SEQ ID NO: 57, and (c) an anti-CD28 scFv comprising VH and VL domains of an anti-CD28 antibody comprising or consisting of SEQ ID NOs: 44 and 45, respectively, optionally with one, two or three modification(s) selected from substitution(s), addition(s), deletion(s) and any combination thereof, preferably linked by a peptide linker, preferably of SEQ ID NO: 57, and
a third chain that is a light chain of an anti-PD1 antibody and comprising or consisting of, VL and CL domains comprising or consisting of SEQ ID NOs: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 25, optionally with one, two or three modification(s) selected from substitution(s), addition(s), deletion(s) and any combination thereof
a second chain that comprises or consists of the sequence as set forth in SEQ ID NO: 61, optionally with one, two or three modification(s) selected from substitution(s), addition(s), deletion(s) and any combination thereof, and
a third chain that comprises or consists of the sequence as set forth in SEQ ID NO: 20 optionally with one, two or three modification(s) selected from substitution(s), addition(s), deletion(s) and any combination thereof.

In a third aspect, the multifunctional molecule comprises or consists of:
a first binding moiety that is an anti-PD1 F(ab′)2;
a second binding moiety that is an agonistic anti-CD28 scFV;
a first and second Fc chains, said first and second Fc chains being complementary and forming a Fc domain,
   wherein the anti-PD1 F(ab′)2 is linked to the Fc domain.

Preferably, the F(ab′)2 comprises one Fab and one CrossMAb, one chain of each being linked to a Fc chain.

In other words, the multifunctional molecule may comprise or consists of:
a first binding moiety that is an anti-PD1 antibody; and
a second binding moiety that is an anti-CD28 scFV linked to the anti-PD1 antibody, optionally by a peptide linker.

Preferably, the anti-PD1 antibody comprises a CrossMAb, preferably one CrossMAb.

Optionally, the anti-CD28 scFV is linked to
a) the C-terminal end of a light chain, preferably one light chain;
b) the N-terminal end of a light chain, preferably one light chain; or
c) the C-terminal end of a heavy chain, preferably one heavy chain.

In one embodiment, said multifunctional molecule particularly comprises or consists of:
a first chain comprising or consisting of, from N-terminus to C-terminus, (i) VL and CH1 domain of an anti-PD1 antibody, (ii) optionally a peptide linker, and (iii) an anti-CD28 scFv comprising VH and VL domains of an anti-CD28 antibody, preferably linked by a peptide linker,
a second chain comprising or consisting of, from N-terminus to C-terminus, VH and CL domains of an anti-PD1 antibody, optionally a hinge, CH2 and CH3 domains of an antibody, said CH2 and CH3 domains forming the first Fc chain,
a third chain comprising or consisting of, from N-terminus to C-terminus, VH and CH1 domains of an anti-PD1 antibody, optionally a hinge, CH2 and CH3 domains of an antibody, said CH2 and CH3 domains forming the second Fc chain,
a fourth chain comprising or consisting of, from N-terminus to C-terminus, (i) VL and CL domains of an anti-PD1 antibody;
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably said multifunctional molecule comprises or consists of:
a first chain that comprises or consists of (i) a chain comprising, from N-terminus to C-terminus, VL and CH1 domains of an anti-PD1 antibody comprising or consisting of SEQ ID NOs: 16 and 17, respectively, optionally with one, two or three modification(s) selected from substitution(s), addition(s), deletion(s) and any combination thereof (ii) optionally a peptide linker, preferably of SEQ ID NO: 57 (iii) an anti-CD28 scFV comprising VH and VL domains of an anti-CD28 antibody comprising or consisting of SEQ ID NOs: 44 and 45, respectively, optionally with one, two or three modification(s) selected from substitution(s), addition(s), deletion(s) and any combination thereof, preferably linked by a peptide linker, preferably of SEQ ID NO: 57;
a second chain that comprises or consists of a chain comprising, from N-terminus to C-terminus, (i) VH and CL domains of an anti-PD1 antibody comprising or consisting of SEQ ID NOs: 15 and 18 respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that comprises or consists of a heavy chain of an anti-PD1 antibody and comprising (i) VH and CH1 domains comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the second Fc chain comprising or consisting of SEQ ID NO: 26, optionally with one, two or three modification(s) selected from substitution(s), addition(s), deletion(s) and any combination thereof;
a fourth chain that comprises or consists of (i) a light chain of an anti-PD1 antibody comprising: VL and CL domains comprising or consisting of SEQ ID NO: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 60, optionally with one, two or three modification(s) selected from substitution(s), addition(s), deletion(s) and any combination thereof
a second chain that comprises or consists of the sequence as set forth in SEQ ID NO: 28, optionally with one, two or three modification(s) selected from substitution(s), addition(s), deletion(s) and any combination thereof
a third chain that comprises or consists of the sequence as set forth in SEQ ID NO: 29, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

In one embodiment, said multifunctional molecule particularly comprises or consists of:
a first chain comprising or consisting of, from N-terminus to C-terminus, (i) an anti-CD28 scFv comprising VH and VL domains of an anti-CD28 antibody, preferably linked by a peptide linker (ii) optionally a peptide linker and (iii) VL and CH1 domains of an anti-PD1 antibody,
a second chain comprising or consisting of, from N-terminus to C-terminus, VH and CL domains of an anti-PD1 antibody, optionally a hinge, CH2 and CH3 domains of an antibody, said CH2 and CH3 domains forming the first Fc chain,
a third chain comprising or consisting of, from N-terminus to C-terminus, VH and CH1 domains of an anti-PD1 antibody, optionally a hinge, CH2 and CH3 domains of an antibody, said CH2 and CH3 domains forming the second Fc chain,
a fourth chain comprising or consisting of, from N-terminus to C-terminus, (i) VL and CL domains of an anti-PD1 antibody;
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably, said multifunctional molecule comprises or consists of:
a first chain that comprises or consists of (i) an anti-CD28 scFV comprising VH and VL domains comprising or consisting of SEQ ID NOs: 44 and 45, respectively, optionally with one, two or three modification(s) selected from substitution(s), addition(s), deletion(s) and any combination thereof, preferably linked by a peptide linker, preferably of SEQ ID NO: 57 (ii) optionally a peptide linker, preferably of SEQ ID NO: 57 (iii) a chain comprising, from N-terminus to C-terminus, VL and CH1 domains of an anti-PD1 antibody comprising or consisting of SEQ ID NOs: 16 and 17, respectively, optionally with one, two or three modification(s) selected from substitution(s), addition(s), deletion(s) and any combination thereof;
a second chain that comprises or consists of a chain comprising, from N-terminus to C-terminus, (i) VH and CL domains of an anti-PD1 antibody comprising or consisting of SEQ ID NOs: 15 and 18, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that comprises or consists of a heavy chain of an anti-PD1 antibody comprising, from N-terminus to C-terminus, (i) VH and CH1 domains comprising or consisting of SEQ ID NO: 15 and 17, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domain of an antibody, said CH2 and CH3 domains forming the second Fc chain comprising or consisting of SEQ ID NO: 26, optionally with one, two or three modification(s) selected from substitution(s), addition(s), deletion(s) and any combination thereof; and
a fourth chain that comprises or consists of (i) a light chain of an anti-PD1 antibody comprising, from N-terminus to C-terminus, VL and CL domains comprising or consisting of SEQ ID NO: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 59, optionally with one, two or three modification(s) selected from substitution(s), addition(s), deletion(s) and any combination thereof
a second chain that comprises or consists of the sequence as set forth in SEQ ID NO: 28, optionally with one, two or three modification(s) selected from substitution(s), addition(s), deletion(s) and any combination thereof
a third chain that comprises or consists of the sequence as set forth in SEQ ID NO: 29, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

In one embodiment, the multifunctional molecule particularly comprises or consists of:
a first chain comprising or consisting of, from N-terminus to C-terminus, (i) VL and CH1 domains of an anti-PD1 antibody, (ii) optionally a peptide linker and (iii) an anti-CD28 scFv comprising VL and VH domains of an anti-CD28 antibody, preferably linked by a peptide linker,
a second chain comprising or consisting of, from N-terminus to C-terminus, VH and CL domains of an anti-PD1 antibody, optionally a hinge, CH2 and CH3 domains of an antibody, said CH2 and CH3 domains forming the first Fc chain,
a third chain comprising or consisting of, from N-terminus to C-terminus, VH and CH1 domains of an anti-PD1 antibody, optionally a hinge, CH2 and CH3 domains of an antibody, said CH2 and CH3 domains forming the second Fc chain, and
a fourth chain comprising or consisting of, from N-terminus to C-terminus, (i) VL and CL domains of an anti-PD1 antibody;
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably, the multifunctional molecule comprises or consists of:
a first chain that comprises or consists of (i) a chain comprising, from N-terminus to C-terminus, VL and CH1 domains of an anti-PD1 antibody comprising or consisting of SEQ ID NOs: 16 and 17, respectively, optionally with one, two or three modification(s) selected from substitution(s), addition(s), deletion(s) and any combination thereof (ii) optionally a peptide linker, preferably of SEQ ID NO: 57 and (iii) an anti-CD28 scFV comprising VL and VH domains comprising or consisting of SEQ ID NOs: 45 and 44, respectively, optionally with one, two or three modification(s) selected from substitution(s), addition(s), deletion(s) and any combination thereof, preferably linked by a peptide linker, preferably of SEQ ID NO: 57;
a second chain that comprises or consists of a chain comprising, from N-terminus to C-terminus, (i) VH and CL domains of an anti-PD1 antibody comprising or consisting of SEQ ID NOs: 15 and 18, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domain of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that comprises or consists of a heavy chain and comprising, from N-terminus to C-terminus, (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof and (iii) CH2 and CH3 domains of an antibody, said CH2 and CH3 domains forming the second Fc chain comprising or consisting of SEQ ID NO: 26, optionally with one, two or three modification(s) selected from substitution(s), addition(s), deletion(s) and any combination thereof; and
a fourth chain that comprises or consists of (i) a light chain of an anti-PD1 antibody comprising, from N-terminus to C-terminus, VL and CL domain of an anti-PD1 antibody comprising or consisting of SEQ ID NO: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 64, optionally with one, two or three modification(s) selected from substitution(s), addition(s), deletion(s) and any combination thereof
a second chain that comprises or consists of the sequence as set forth in SEQ ID NO: 28, optionally with one, two or three modification(s) selected from substitution(s), addition(s), deletion(s) and any combination thereof
a third chain that comprises or consists of the sequence as set forth in SEQ ID NO: 29, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

In one embodiment, said multifunctional molecule particularly comprises or consists of:
a first chain comprising or consisting of, from N-terminus to C-terminus, (i) an anti-CD28 scFv comprising VL and VH domains of an anti-CD28 antibody, preferably linked by a peptide linker, (ii) optionally a peptide linker, (iii) VL and CH1 domains of an anti-PD1 antibody,
a second chain comprising or consisting of, from N-terminus to C-terminus, VH and CL of an anti-PD1 antibody, optionally a hinge, CH2 and CH3 domains of an antibody, said CH2 and CH3 domains forming the first Fc chain,
a third chain comprising or consisting of, from N-terminus to C-terminus, VH and CH1 of an anti-PD1 antibody, optionally a hinge, CH2 and CH3 domains of an antibody, said CH2 and CH3 domains forming the second Fc chain, and
a fourth chain comprising or consisting of, from N-terminus to C-terminus, (i) VL and CL domains of an anti-PD1 antibody;
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably said multifunctional molecule comprises or consists of:
a first chain that comprises or consists of (i) an anti-CD28 scFV comprising VL and VH domains comprising or consisting of SEQ ID NOs: 45 and 44, respectively, optionally with one, two or three modification(s) selected from substitution(s), addition(s), deletion(s) and any combination thereof, preferably linked by a peptide linker, preferably of SEQ ID NO: 57 (ii) optionally a peptide linker, preferably of SEQ ID NO: 57, and (iii) a chain comprising, from N-terminus to C-terminus, VL and CH1 domains of an anti-PD1 antibody comprising or consisting of SEQ ID NOs: 16 and 17, respectively, optionally with one, two or three modification(s) selected from substitution(s), addition(s), deletion(s) and any combination thereof;
a second chain that comprises or consists of a chain comprising, from N-terminus to C-terminus, (i) VH and CL domains of an anti-PD1 antibody comprising or consisting of SEQ ID NOs: 15 and 18, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof and (iii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain that comprises or consists of a heavy chain of an anti-PD1 antibody and comprising, from N-terminus to C-terminus, (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof and (iii) CH2 and CH3 domains of an antibody, said CH2 and CH3 domains forming the second Fc chain comprising or consisting of SEQ ID NO: 26, optionally with one, two or three modification(s) selected from substitution(s), addition(s), deletion(s) and any combination thereof; and
a fourth chain that comprises or consists of (i) a light chain of an anti-PD1 antibody comprising, from N-terminus to C-terminus, VL and CL domains of an anti-PD1 antibody, comprising or consisting of SEQ ID NOs: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 63, optionally with one, two or three modification(s) selected from substitution(s), addition(s), deletion(s) and any combination thereof
a second chain that comprises or consists of the sequence as set forth in SEQ ID NO: 28, optionally with one, two or three modification(s) selected from substitution(s), addition(s), deletion(s) and any combination thereof
a third chain that comprises or consists of the sequence as set forth in SEQ ID NO: 29, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

In another embodiment, said multifunctional molecule particularly comprises or consists of:
a first chain comprising or consisting of, from N-terminus to C-terminus, VL and CL domains of an anti-PD1 antibody;
a second chain comprising or consisting of, from N-terminus to C-terminus, VH and CH1 domains of an anti-PD1 antibody, a hinge, CH2 and CH3 domains of an antibody, the CH2 and CH3 domains forming the first Fc chain;
a third chain comprising or consisting of, from N-terminus to C-terminus, (i) VH and CH1 domains of an anti-PD1 antibody, a hinge, CH2 and CH3 domains of an antibody, the CH2 and CH3 domains forming the second Fc chain, optionally a peptide linker and (ii) VL and VH domains of an anti-CD28 antibody, preferably linked by a peptide linker; and
a fourth chain comprising or consisting of, from N-terminus to C-terminus, VL and CL domains of an anti-PD1 antibody, wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably, said multifunctional molecule comprises or consists of:
a first chain that is a light chain comprising or consisting of, from N-terminus to C-terminus, VL and CL domains of an anti-PD1 antibody comprising or consisting of SEQ ID NOs: 16 and 18, respectively, optionally with one, two or three modification(s) selected from substitution(s), addition(s), deletion(s) and any combination thereof;
a second chain that is a heavy chain comprising or consisting of, from N-terminus to C-terminus, (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof;
a third chain comprising or consisting of, from N-terminus to C-terminus, a) a heavy chain comprising: (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof and (iii) CH2 and CH3 domains of an antibody, said CH2 and CH3 domains forming the second Fc chain comprising or consisting of SEQ ID NO: 26, optionally with one, two or three modification(s) selected from substitution(s), addition(s), deletion(s) and any combination thereof; (b) optionally a peptide linker, preferably of SEQ ID NO: 57 and (c) an anti-CD28 scFv comprising VL and VH domains of an anti-CD28 antibody comprising or consisting of SEQ ID NOs: 45 and 44, respectively, optionally with one, two or three modification(s) selected from substitution(s), addition(s), deletion(s) and any combination thereof, preferably linked by a peptide linker, preferably of SEQ ID NO: 57, and
a fourth chain that is a light chain of an anti-PD1 antibody comprising or consisting of, from N-terminus to C-terminus, VL and CL domains of an anti-PD1 antibody comprising or consisting of SEQ ID NO: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 20, optionally with one, two or three modification(s) selected from substitution(s), addition(s), deletion(s) and any combination thereof
a second chain that comprises or consists of the sequence as set forth in SEQ ID NO: 27, optionally with one, two or three modification(s) selected from substitution(s), addition(s), deletion(s) and any combination thereof
a third chain that comprises or consists of the sequence as set forth in SEQ ID NO: 62, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

In another embodiment, said multifunctional molecule particularly comprises or consists of:
a first chain comprising or consisting of, from N-terminus to C-terminus, VL and CL domains of an anti-PD1 antibody
a second chain comprising or consisting of, from N-terminus to C-terminus, VH and CH1 domains of an anti-PD1 antibody, a hinge, CH2 and CH3 domains of an antibody, the CH2 and CH3 domains forming the first Fc chain,
a third chain comprising or consisting of, from N-terminus to C-terminus, (i) VH and CH1 domains of an anti-PD1 antibody, a hinge, CH2 and CH3 domains of an antibody, the CH2 and CH3 domains forming the second Fc chain, optionally a peptide linker, and (ii) VH and VL domains of an anti-CD28 antibody, preferably linked by a peptide linker;
a fourth chain comprising or consisting of, from N-terminus to C-terminus, VL and CL domains of an anti-PD1 antibody,
wherein the first and second Fc chains are complementary and thereby form a Fc domain.

In particular, the first Fc chain is a “knob” and the second Fc chain is a “hole”. Alternatively, the first Fc chain is a “hole” and the second Fc chain is a “knob”.

Preferably, said multifunctional molecule comprises or consists of:
a first chain that is a light chain of an anti-PD1 antibody and comprising or consisting of, from N-terminus to C-terminus, VL and CL domains of an anti-PD1 antibody comprising or consisting of SEQ ID NOs: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof and (iii) CH2 and CH3 domains of an antibody, said CH2 and CH3 domains forming the first Fc chain comprising or consisting of SEQ ID NO: 25, optionally with one, two or three modification(s) selected from substitution(s), addition(s), deletion(s) and any combination thereof;
a third chain comprising or consisting of a) a heavy chain comprising, from N-terminus to C-terminus, (i) VH and CH1 domains of an anti-PD1 antibody comprising or consisting of SEQ ID NOs: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, said CH2 and CH3 domains forming the second Fc chain comprising or consisting of SEQ ID NO: 26, optionally with one, two or three modification(s) selected from substitution(s), addition(s), deletion(s) and any combination thereof; (b) optionally a peptide linker, preferably of SEQ ID NO: 57 and (c) an anti-CD28 scFv comprising VH and VL domains of an anti-CD28 antibody comprising or consisting of SEQ ID NOs: 44 and 45, respectively, optionally with one, two or three modification(s) selected from substitution(s), addition(s), deletion(s) and any combination thereof, preferably linked by a peptide linker, preferably of SEQ ID NO: 57,
a fourth chain that is a light chain of an anti-PD1 antibody and comprising or consisting of, from N-terminus to C-terminus, VL and CL domains of an anti-PD1 antibody comprising or consisting of SEQ ID NOs: 16 and 18, respectively, optionally with one, two or three modification(s) selected from substitution(s), addition(s), deletion(s) and any combination thereof;
wherein the first and second Fc chains are complementary and thereby form a Fc domain.

In a very particular embodiment, said multifunctional molecule comprises or consists of:
a first chain that comprises or consists of the sequence as set forth in SEQ ID NO: 20, optionally with one, two or three modification(s) selected from substitution(s), addition(s), deletion(s) and any combination thereof
a second chain that comprises or consists of the sequence as set forth in SEQ ID NO: 27, optionally with one, two or three modification(s) selected from substitution(s), addition(s), deletion(s) and any combination thereof
a third chain that comprises or consists of the sequence as set forth in SEQ ID NO: 61, optionally with one, two or three modification(s) selected from substitution(s), addition(s), deletion(s) and any combination thereof, and
a fourth chain that comprises or consists of the sequence as set forth in SEQ ID NO: 20, optionally with one, two or three modification(s) selected from substitution(s), addition(s), deletion(s) and any combination thereof.

Each of the above-described molecule comprises a single (one) anti-CD28 binding moiety.

Preferably, when mutations are in the VL or VH, the one, two or three modification(s) are preferably outside of the CDRs (i.e., are in the framework regions).

In some embodiments, the anti-CD28 binding domain is a variant comprising one or more mutations in the CDRs and/or framework region. Said anti-CD28 variant are particularly described above in the section “Second binding moiety”. Any one of said variants can be incorporated in the above exemplified multifunctional molecule.

In some aspects, the multifunctional molecule comprises or consists of:
a first chain that is a light chain of an anti-PD1 antibody and comprising or consisting of, from N-terminus to C-terminus, VL and CL domains comprising or consisting of SEQ ID Nos: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID Nos: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof; and
a third chain comprising or consisting of, from N-terminus to C-terminus, (i) an anti-CD28 scFv comprising:
(i) (a) a heavy chain variable region (VH) comprising or consisting of an amino acid sequence selected from the group consisting of SEQ ID NO: 44, 83, 84, 85 and 88; optionally with one, two or three modification(s) selected from substitution(s), addition(s), deletion(s) and any combination thereof; and
(b) a light chain variable region (VL) comprising or consisting of an amino acid sequence selected from the group consisting of SEQ ID NO: 45, 86, 87, 89 and 90, optionally with one, two or three modification(s) selected from substitution(s), addition(s), deletion(s) and any combination thereof; wherein the VH and VL are preferably linked by a peptide linker, preferably of SEQ ID NO: 57;
(ii) optionally a peptide linker, preferably of SEQ ID NO: 57, (iii) optionally a hinge, preferably of SEQ ID NO: 58, optionally with one, two or three modification(s) selected from substitution(s), addition(s), deletion(s) and any combination thereof iv) CH2 and CH3 domains of an antibody, the CH2 and CH3 domains forming the second Fc chain comprising or consisting of SEQ ID NO: 26 or 54, preferably of SEQ ID NO: 54 optionally with one, two or three modification(s) selected from substitution(s), addition(s), deletion(s) and any combination thereof;
wherein the first and second Fc chains are complementary and thereby form a Fc domain.

In some aspects, the multifunctional molecule comprises or consists of:
a first chain that is a light chain of an anti-PD1 antibody and comprising or consisting of, from N-terminus to C-terminus, VL and CL domains comprising or consisting of SEQ ID Nos: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID Nos: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof; and
a third chain comprising or consisting of, from N-terminus to C-terminus, (i) an anti-CD28 scFv comprising:
(a) a heavy chain variable region (VH) comprising or consisting of an amino acid sequence selected from the group consisting of SEQ ID NO: 44, 83, 84, 85 and 88; and
(b) a light chain variable region (VL) comprising or consisting of an amino acid sequence of SEQ ID NO: 45, optionally with 1, 2 or 3 modification(s) selected from substitution(s), addition(s), deletion(s) and any combination thereof, preferably amino acid substitutions, wherein the VH and VL are preferably linked by a peptide linker, preferably of SEQ ID NO: 57;
(ii) optionally a peptide linker, preferably of SEQ ID NO: 57, (iii) optionally a hinge, preferably of SEQ ID NO: 58, optionally with one, two or three modification(s) selected from substitution(s), addition(s), deletion(s) and any combination thereof iv) CH2 and CH3 domains of an antibody, the CH2 and CH3 domains forming the second Fc chain comprising or consisting of SEQ ID NO: 26 or 54, preferably of SEQ ID NO: 54 optionally with one, two or three modification(s) selected from substitution(s), addition(s), deletion(s) and any combination thereof;
wherein the first and second Fc chains are complementary and thereby form a Fc domain.

In some aspects, the multifunctional molecule comprises or consists of:
a first chain that is a light chain of an anti-PD1 antibody and comprising or consisting of, from N-terminus to C-terminus, VL and CL domains comprising or consisting of SEQ ID Nos: 16 and 18, respectively, optionally with one, two or three modification(s) selected from substitution(s), addition(s), deletion(s) and any combination thereof;
a second chain that is a heavy chain of an anti-PD1 antibody and comprising or consisting of, from N-terminus to C-terminus, (i) VH and CH1 domains comprising or consisting of SEQ ID Nos: 15 and 17, respectively, optionally with one, two or three modification(s) selected from substitution(s), addition(s), deletion(s) and any combination thereof; (ii) optionally a hinge, preferably of SEQ ID NO: 58 optionally with one, two or three modification(s) selected from substitution(s), addition(s), deletion(s) and any combination thereof (iii) CH2 and CH3 domains of an antibody, the CH2 and CH3 domains forming the first Fc chain comprising or consisting of SEQ ID NO: 25, optionally with one, two or three modification(s) selected from substitution(s), addition(s), deletion(s) and any combination thereof; and
a third chain comprising or consisting of, from N-terminus to C-terminus, (i) an anti-CD28 scFv comprising:
(a) a heavy chain variable region (VH) comprising or consisting of an amino acid sequence of SEQ ID NO: 44 optionally with 1, 2 or 3 modification(s) selected from substitution(s), addition(s), deletion(s) and any combination thereof, preferably amino acid substitutions; and (b) a light chain variable region (VL) comprising or consisting of an amino acid sequence selected from the group consisting of SEQ ID NO: 45, 86, 87, 89 and 90, wherein the VH and VL are preferably linked by a peptide linker, preferably of SEQ ID NO: 57;
(ii) optionally a peptide linker, preferably of SEQ ID NO: 57, (iii) optionally a hinge, preferably of SEQ ID NO: 58, optionally with one, two or three modification(s) selected from substitution(s), addition(s), deletion(s) and any combination thereof iv) CH2 and CH3 domains of an antibody, the CH2 and CH3 domains forming the second Fc chain comprising or consisting of SEQ ID NO: 26 or 54, preferably of SEQ ID NO: 54 optionally with one, two or three modification(s) selected from substitution(s), addition(s), deletion(s) and any combination thereof;
wherein the first and second Fc chains are complementary and thereby form a Fc domain.

### Preparation of Multifunctional Molecule—Nucleic Acid Molecules Encoding the Multifunctional Molecules of the Present Invention, Recombinant Expression Vectors and Host Cells Comprising Such

To produce a multifunctional molecule according to the invention, in particular by mammalian cells, nucleic acid sequences or group of nucleic acid sequences coding for the multifunctional molecule are subcloned into one or more expression vectors. Such vectors are generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:
(1) transfecting or transforming appropriate host cells with the polynucleotide(s) encoding the recombinant multifunctional molecule of the invention or the vector containing the polynucleotide(s);
(2) culturing the host cells in an appropriate medium; and
(3) optionally isolating and/or purifying the multifunctional molecule from the medium or host cells.

The invention further relates to a nucleic acid encoding a multifunctional molecule as disclosed above, a vector, preferably an expression vector, comprising the nucleic acid of the invention, a genetically engineered host cell transformed with the vector of the invention or directly with the sequence encoding the recombinant multifunctional molecule, and a method for producing the multifunctional molecule of the invention by recombinant techniques.

The nucleic acid, the vector and the host cells are more particularly described hereafter.

### Nucleic Acid Sequence

The invention also relates to a nucleic acid molecule encoding the multifunctional molecule as defined above or to a group of nucleic acid molecules encoding the multifunctional molecule as defined above. Nucleic acid encoding the multifunctional molecule disclosed herein can be amplified by any techniques known in the art, such as PCR. Such nucleic acid may be readily isolated and sequenced using conventional procedures know by the man of the art.

Preferably, the invention relates to an isolated nucleic acid molecule encoding the multifunctional molecule as defined above or to a group of isolated nucleic acid molecules encoding the multifunctional molecule as defined above.

In a particular aspect, the nucleic acid molecules encoding the multifunctional molecule as defined herein comprises or consists of nucleic acid molecule or group of nucleic acid molecules encoding the different chains of the multifunctional molecules described herein, in particular under the section “Examples of multifunctional molecules”.

In one embodiment, the nucleic acid molecule is an isolated, particularly non-natural, nucleic acid molecule.

### Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The nucleic acid molecule or group of nucleic acid molecules encoding the multifunctional molecule can be cloned into a vector by those skilled in the art, and then transformed into host cells. These methods include in vitro recombinant DNA techniques, DNA synthesis techniques, in vivo recombinant techniques, etc. The methods known to the artisans in the art can be used to construct an expression vector containing the nucleic acid sequence of the multifunctional molecule, variant described herein and appropriate regulatory components for transcription/translation.

Accordingly, the present invention also provides a recombinant vector, which comprises a nucleic acid molecule or group of nucleic acid molecules encoding the multifunctional molecule according to the present invention. In one preferred aspect, the expression vector further comprises a promoter and a nucleic acid sequence encoding a secretion signal peptide, and optionally at least one drug-resistance gene for screening. The expression vector may further comprise a ribosome-binding site for initiating the translation, transcription terminator and the like.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants.

### Host Cells

In another aspect, the invention relates to a host cell comprising a vector or a nucleic acid molecule or group of nucleic acid molecules as defined above, for example for multifunctional molecule production purposes.

As used herein, the term “host cell” is intended to include any individual cell or cell culture that can be or has been recipient of vectors, exogenous nucleic acid molecules, and polynucleotides encoding the multifunctional molecule according to the present invention. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

Suitable hosts cells are especially eukaryotic hosts cells which provide suitable post-translational modifications such as glycosylation. Preferably, such suitable eukaryotic host cell may be fungi such as Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells.

Preferably, the host cell of the present invention is selected from the group consisting of CHO cell, COS cell, NSO cell, and HEK cell.

Then host cells stably or transiently express the multifunctional molecule according to the present invention. Such expression methods are known by the man skilled in the art.

A method of production of the multifunctional molecule is also disclosed herein. The method comprises culturing a host cell comprising a nucleic acid or a group of nucleic acids encoding the multifunctional molecule as provided above, under conditions suitable for its expression, and optionally recovering the multifunctional molecule from the host cell (or host cell culture medium). Particularly, for recombinant production of a multifunctional molecule, nucleic acids or group of nucleic acids encoding a multifunctional molecule, e.g., as described above, are isolated and inserted into one or more vectors for further cloning and/or expression in a host cell. The multifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. Multifunctional molecule isolation techniques may particularly include affinity chromatography with Protein-A Sepharose, size-exclusion chromatography and ion exchange chromatography. Protein A preferably is used to isolate the multifunctional molecules of the invention.

### Method of Selecting a Suitable Multifunctional Molecule

In an aspect, the invention relates to a method of selecting a multifunctional molecule of the invention, comprising or consisting of at least one of the following steps:
a. selecting the first binding moiety that binds to a target specifically expressed on immune cells surface; and wherein the target specifically expressed on immune cells surface is preferably selected from the group consisting of PD-1, CTLA-4, BTLA, TIGIT, CD160, CD40L, ICOS, CD27, OX40, 4-1BB, GITR, HVEM, Tim-1, LFA-1, TIM3, CD39, CD30, NKG2D, NKG2A, LAG3, 2B4, DR3, CD101, CD44, CD38, CXCR3, CXCR5, CD4, CD8, CD25, CRTAM, CD96, CD226, CD112R, CD103, CEACAM and CD122, preferably from the group consisting of PD-1, VISTA, CTLA-4, BTLA, TIGIT, CD160, LAG3 and TIM3 and is preferably PD-1
b. selecting the structure of the multifunctional molecule;
c. constructing/cloning the multifunctional molecule comprising the anti-CD28 binding domain and the first binding moiety;
d. testing the ability of the multifunctional molecule to bind to PD-1, CTLA-4, BTLA, TIGIT, CD160, CD40L, ICOS, CD27, OX40, 4-1BB, GITR, HVEM, Tim-1, LFA-1, TIM3, CD39, CD30, NKG2D, NKG2A, LAG3, 2B4, DR3, CD101, CD44, CD38, CXCR3, CXCR5, CD4, CD8, CD25, CRTAM, CD96, CD226, CD112R, CD103, CEACAM and CD122, preferably from the group consisting of PD-1, VISTA, CTLA-4, BTLA, TIGIT, CD160, LAG3 and TIM3, more preferably to PD-1 and optionally the ability of the multifunctional molecule to inhibit the binding of human PD-L1 and/or PD-L2 to human PD-1;
e. testing the ability of the multifunctional molecule to bind CD28 and the ability of the multifunctional to have an agonistic activity on CD28;
f. testing:
   ii) the ability of a multifunctional molecule of cis-binding to an immune cell, in particular a T cell; and/or
   iii) the ability of a multifunctional molecule to not exhibit trans-activity, i.e., co-targeting tumor cells and T cells;
      and optionally comprising the following step:
g. selecting a multifunctional molecule which specifically binds to the target specifically expressed on immune cells surface is preferably selected from the group consisting of PD-1, CTLA-4, BTLA, TIGIT, CD160, CD40L, ICOS, CD27, OX40, 4-1BB, GITR, HVEM, Tim-1, LFA-1, TIM3, CD39, CD30, NKG2D, NKG2A, LAG3, 2B4, DR3, CD101, CD44, CD38, CXCR3, CXCR5, CD4, CD8, CD25, CRTAM, CD96, CD226, CD112R, CD103, CEACAM and CD122, preferably from the group consisting of PD-1, VISTA, CTLA-4, BTLA, TIGIT, CD160, LAG3 and TIM3, more preferably of PD1; and to CD28 and is able of cis-binding to an immune cell, in particular a T cell, and/or does not exhibit trans-activity.

The method of selecting a multifunctional molecule of the invention can advantageously be performed further to the method of manufacturing a multifunctional molecule according to the invention as described hereabove.

### Pharmaceutical Composition and Method of Administration Thereof

The present invention also relates to a pharmaceutical composition comprising a multifunctional molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers, excipients, salts, anti-oxidant and/or stabilizers which do not deleteriously interact with the multifunctional molecule of the invention, nucleic acid, vector and/or host cell of the invention and does not impart any undesired toxicological effects. Optionally, the pharmaceutical composition may further comprise an additional therapeutic agent.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the multifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

The pharmaceutical composition may be prepared by mixing a multifunctional molecule having the desired degree of purity with optional pharmaceutically acceptable carriers, excipients, anti-oxidant, and/or stabilizers in the form of lyophilized formulations or aqueous solutions. Such suitable carriers, excipients, anti-oxidant, and/or stabilizers are well known in the art and have been for example described in Remington's Pharmaceutical Sciences 16th edition, Osol, A. Ed. (1980).

To facilitate delivery, any of the multifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polypeptide.

Pharmaceutical compositions according to the invention may be formulated to release the active ingredients (e.g., the multifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Preferably, pharmaceutical compositions described herein will generally be administered in such amounts and for such a time as is necessary or sufficient to induce an immune response.

### Use in the Treatment of a Disease

The multifunctional molecules, nucleic acids, vectors, host cells, compositions and methods of the present invention have numerous in vitro and in vivo utilities and applications. Particularly, any of multifunctional molecules, nucleic acid molecules, group of nucleic acid molecules, vectors, host cells or pharmaceutical composition provided herein may be used in therapeutic methods and/or for therapeutic purposes.

The present invention also relates to a multifunctional molecule, a nucleic acid or a vector encoding such, or a pharmaceutical composition comprising such for use in the treatment of a disorder and/or disease in a subject and/or for use as a medicament or vaccine. It also relates to the use of a multifunctional molecule as described herein; a nucleic acid or a vector encoding such, or a pharmaceutical composition comprising such for treating a disease and/or disorder in a subject. It also relates to the use of a multifunctional molecule as described herein; a nucleic acid or a vector encoding such, or a pharmaceutical composition comprising in the manufacture of a medicament for the treatment of a disease and/or disorder in a subject. Finally, it relates to a method for treating a disease or a disorder in a subject comprising administering a therapeutically effective amount of a pharmaceutical composition or a multifunctional molecule to the subject, or a nucleic acid or a vector encoding such.

The use of the multifunctional molecule, nucleic acid, group of nucleic acids, vector encoding such, or pharmaceutical composition comprising such according to the invention has applications in all T lymphocyte-dependent pathological conditions.

Such pathological conditions can for example be cancer or infectious diseases.

In one aspect, the invention relates to a method of treatment of a disease and/or disorder selected from the group consisting of a cancer and an infectious disease, in a subject in need thereof comprising administering to said subject an effective amount of a multifunctional molecule or pharmaceutical composition as defined above. Examples of such diseases are more particularly described hereafter.

In one aspect, the treatment method comprises: (a) identifying a patient in need of treatment; and (b) administering to the patient a therapeutically effective amount of a multifunctional molecule, nucleic acid, group of nucleic acids, vector or pharmaceutical composition as described herein.

A subject in need of a treatment may be a human having, at risk for, or suspected of having a disease. Such a patient can be identified by routine medical examination.

In another aspect, the multifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a multifunctional molecule, nucleic acid, vector or pharmaceutical composition of the invention such that the immune response in the subject is modified. Preferably, the immune response is enhanced, increased, stimulated or up-regulated.

### Cancer

In another aspect, the invention provides the use of a multifunctional molecule or pharmaceutical composition as disclosed herein in the manufacture of a medicament for treating a cancer, for instance for inhibiting growth of tumor cells in a subject.

The invention also provides a multifunctional molecule, a combination, or a pharmaceutical composition for use in the treatment of a subject having a cancer.

The invention also provides a method for treating cancer, in a subject comprising administering a therapeutically effective amount of a pharmaceutical composition, a combination or a multifunctional molecule such as disclosed herein.

The term “cancer” as used herein is defined as disease characterized by the rapid and uncontrolled growth of aberrant cells. Cancer cells can spread locally or through the bloodstream and lymphatic system to other parts of the body.

Accordingly, in one aspect, the invention provides a method of treating a cancer, for instance for inhibiting growth of tumor cells, in a subject, comprising administering to the subject a therapeutically effective amount of multifunctional molecule or pharmaceutical composition according to the invention. Particularly, the present invention relates to the treatment of a subject using a multifunctional molecule such that growth of cancerous cells is inhibited.

Any suitable cancer may be treated with the provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. Additionally, the invention includes refractory or recurrent malignancies. Preferably, the cancer to be treated or prevented is selected from the group consisting of metastatic or not metastatic, Melanoma, malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

In a particular aspect, the cancer is a hematologic malignancy or a solid tumor. Such a cancer can be selected from the group consisting of hematolymphoid neoplasms, angioimmunoblastic T cell lymphoma, myelodysplastic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyngeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferred cancers for treatment include cancers typically responsive to immunotherapy. Alternatively, preferred cancers for treatment are cancers non-responsive to immunotherapy.

### Infectious Disease

The multifunctional molecule, nucleic acid, group of nucleic acid, vector, host cells or pharmaceutical compositions of the invention can be used to treat patients that have been exposed to particular toxins or pathogens. Accordingly, an aspect of the invention provides a method of treating an infectious disease in a subject comprising administering to the subject a multifunctional molecule according to the present invention, or a pharmaceutical composition comprising such, preferably such that the subject is treated for the infectious disease.

Any suitable infection may be treated with a multifunctional molecule, nucleic acid, group of nucleic acid, vector, host cells or pharmaceutical composition as provided herein.

Some examples of pathogenic viruses causing infections treatable by methods of the invention include Human immunodeficiency virus (HIV), hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, Human T-lymphotropic virus (HTLV), dengue virus, papillomavirus, molluscum virus, poliovirus, rabies virus, JC virus and arboviral encephalitis virus.

Some examples of pathogenic bacteria causing infections treatable by methods of the invention include chlamydia, rickettsia bacteria, mycobacteria, staphylococci, streptococci, pneumococci, meningococci and gonococci, klebsiella, proteus, serratia, pseudomonas, legionella, diphtheria, salmonella, bacilli, cholera, tetanus, botulism, anthrax, plague, leptospirosis, and Lyme's disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis.

### Combination or Combined Therapy

In an aspect, the invention refers to a combination of a) a multifunctional molecule of the invention and b) a distinct therapeutic agent, preferably an anti-cancer or anti-infectious agent. Such combination may particularly be comprised in a pharmaceutical composition such as described herein.

The multifunctional molecule according to the invention can be combined with some other potential strategies for overcoming immune evasion mechanisms with agents in clinical development or already on the market (see for example table 1 from Antonia et al. Immuno-oncology combinations: a review of clinical experience and future prospects. Clin. Cancer Res. Off. J. Am. Assoc. Cancer Res. 20, 6258-6268, 2014). Such combination with the multifunctional molecule according to the invention may be useful notably for:
1—Reversing the inhibition of adaptive immunity (blocking T-cell checkpoint pathways);
2—Switching on adaptive immunity (promoting T-cell costimulatory receptor signaling using agonist molecules, in particular antibodies),
3—Improving the function of innate immune cells;
4—Activating the immune system (potentiating immune-cell effector function), for example through vaccine-based strategies.

Accordingly, also provided herein are combined therapies with any of the multifunctional molecule or pharmaceutical composition comprising such, as described herein and a suitable second agent, for the treatment of a disease or disorder. In an aspect, the multifunctional molecule and the second agent can be present in a unique (or single) pharmaceutical composition as described above. Alternatively, the terms “combination therapy” or “combined therapy”, as used herein, embrace administration of these two agents (e.g., a multifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a multifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In an aspect, the additional therapeutic agent can be selected in the non-exhaustive list comprising alkylating agents, angiogenesis inhibitors, antibodies, antimetabolites, antimitotic, antiproliferative, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, leukemia viral oncogene homolog (ErbB2) receptor inhibitors, growth factor inhibitors, heat shock protein (HSP)-90 inhibitors, histone deacetylase (HDAC) inhibitors, hormonal therapies, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids, plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

In one aspect, the invention relates to a combined therapy as defined above, wherein the second therapeutic agent is particularly selected from the group consisting of therapeutic vaccines, immune checkpoint blockers or activators, in particular of adaptive immune cells (T and B lymphocytes) and antibody-drug conjugates. Preferably, suitable agents for co-use with any of the multifunctional molecule or with the pharmaceutical composition according to the invention include an antibody binding to a co-stimulatory receptor (e.g., OX40, CD40, ICOS, CD27, HVEM or GITR), an agent that induces immunogenic cell death (e.g., a chemotherapeutic agent, a radio-therapeutic agent, an anti-angiogenic agent, or an agent for targeted therapies), an agent that inhibits a checkpoint molecule (e.g., CTLA4, LAG3, TIM3, BTLA, or TIGIT), a cancer vaccine, an agent that modifies an immunosuppressive enzyme (e.g., IDO1 or iNOS), an agent that targets Treg cells, an agent for adoptive cell therapy, or an agent that modulates myeloid cells.

In an aspect, the invention relates to a combined therapy as defined above, wherein the second therapeutic agent is an immune checkpoint blocker or activator of adaptive immune cells (T and B lymphocytes) selected from the group consisting of anti-CTLA4, anti-CD2, anti-CD40, anti-HVEM, anti-BTLA, anti-CD160, anti-TIGIT, anti-TIM-1/3, anti-LAG-3, anti-2B4, and anti-OX40, anti-CD40 agonist, CD40-L, TLR agonists, anti-ICOS, ICOS-L and B-cell receptor agonists.

The present invention also relates to a method for treating a disease in a subject comprising administering to said subject a therapeutically effective amount of the multifunctional molecule or the pharmaceutical composition described herein and a therapeutically effective amount of an additional or second therapeutic agent.

Specific examples of additional or second therapeutic agents are provided in WO 2018/053106, pages 36-43.

In a preferred aspect, the second therapeutic agent is selected from the group consisting of chemotherapeutic agents, radiotherapy agents, immunotherapeutic agents, cell therapy agents (such as CAR-T cells), antibiotics and probiotics.

Combination therapy could also rely on the combination of the administration of the multifunctional molecule according to the invention or the pharmaceutical composition comprising such with surgery.

Particularly, the multifunctional molecule according to the invention is for use in combination with a second multifunctional molecule comprising at least one antigen binding domain that binds to a target specifically expressed on immune cells surface different from the first multifunctional molecule and/or multifunctional molecule comprising at least one immuno-stimulating cytokine.

The multifunctional molecule of the invention and the second multifunctional molecule are administered either simultaneously or sequentially. In an example of a sequential administration, the multifunctional molecule of the invention is administrated before the administration of the second multifunctional molecule. In another example, the multifunctional molecule of the invention is administrated after the administration of the second multifunctional molecule.

### Subject, Regimen and Administration

The present invention relates to a multifunctional molecule as disclosed herein, a nucleic acid, a group of nucleic acids or a vector encoding such, a host cell or a pharmaceutical composition for use as a medicament or for use in the treatment of a disease or for administration in a subject. It also relates to a method for treating a disease or a disorder in a subject comprising administering a therapeutically effective amount of a pharmaceutical composition or a multifunctional molecule to a subject.

The subject to treat may be a human, particularly a human at the prenatal stage, a new-born, a child, an infant, an adolescent or an adult, in particular an adult of at least 20 years old, 30 years old, 40 years old, preferably an adult of at least 50 years old, still more preferably an adult of at least 60 years old.

In a particular aspect, the subject can be immunosuppressed or immunocompromised. An immunocompromised subject is a subject who is incapable of developing or unlikely to develop a robust immune response, usually as a result of disease, malnutrition, or immunosuppressive therapy. Those who can be considered to be immunosuppressed or immunocompromised include, but are not limited to, subjects who have been treated with or is a candidate for treatment with an immunosuppressant, subjects with AIDS (or HIV positive), subjects with severe combined immunodeficiency, diabetics, subjects who have had transplants and who are taking immunosuppressants, and those who are receiving chemotherapy for cancer. Immunocompromised individuals also include subjects with most forms of cancer (other than skin cancer), sickle cell anemia, cystic fibrosis, those who do not have a spleen, subjects with end stage kidney disease (dialysis), and those who have been taking corticosteroids or other immune suppressing therapy on a frequent basis within the last year.

In some particular embodiments, the subject is affected with a disease that involve the PD-1/PDL-1 pathway, particularly wherein, at least one of the ligands of PD-1 (e.g., PDL-1 and/or PDL-2) or PD-1 is/are expressed, especially overexpressed. Preferably, the subject is suffering from cancer, even more preferably from a PD1, PD-L1 and/or PD-L2 positive cancer or a PD-1 positive cancer.

In a particular embodiment, the subject has already received at least one line of treatment, preferably several lines of treatment, prior to the administration of the multifunctional molecule according to the invention or of a pharmaceutical composition according to the invention.

In some embodiments, the patient has a resistance against the treatment, particularly the anti-cancer treatment said treatment being particularly an immune checkpoint inhibitor such as an anti-PD-1 immunotherapy.

Conventional methods, known to those of ordinary skill in the art of medicine, can be used to administer the multifunctional molecule or the pharmaceutical composition disclosed herein to a subject, depending upon the type of diseases to be treated or the site of the disease e.g., administered orally, parenterally, enterally, by inhalation spray, topically, rectally, nasally, buccally, vaginally or via an implanted reservoir. Preferably, the multifunctional molecule or the pharmaceutical composition is administered via subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques.

The form of the pharmaceutical compositions, the route of administration and the dose of administration of the pharmaceutical composition or the multifunctional molecule according to the invention can be adjusted by the man skilled in the art according to the type and severity of the infection, and to the patient, in particular its age, weight, size, sex, and/or general physical condition. The compositions of the present invention may be administered in a number of ways depending upon whether local or systemic treatment is desired.

### Kits

Any of the multifunctional molecules or compositions described herein may be included in a kit provided by the present invention. The present disclosure particularly provides kits for use in treating diseases or disorders (e.g., cancer and/or infection) In the context of the present invention, the term “kit” means two or more components (one of which corresponding to the multifunctional molecule, the nucleic acid molecule, the vector or the cell of the invention) packaged in a container, recipient or otherwise. A kit can hence be described as a set of products and/or utensils that are sufficient to achieve a certain goal, which can be marketed as a single unit. The kits of this invention are in suitable packaging.

Particularly, a kit according to the invention may comprise:
a multifunctional molecule as defined above,
a pharmaceutical composition comprising said multifunctional molecule,
a nucleic acid molecule or a group of nucleic acid molecules encoding said multifunctional molecule,
a vector comprising said nucleic acid molecule or group of nucleic acid molecules, and/or
a cell comprising said vector or nucleic acid molecule or group of nucleic acid molecules.

The kit may include, in suitable container means, the pharmaceutical composition or multifunctional molecules, and/or host cells of the present invention, and/or vectors encoding the nucleic acids of the present invention.

The components comprised in the kit according to the invention, especially the pharmaceutical composition may particularly be formulated into a syringe compatible composition.

In some embodiments, the kit further includes an additional agent for treating cancer or an infectious disease, and the additional agent may be combined with the pharmaceutical composition, fusion proteins or multifunctional molecules, and/or host cells of the present invention, and/or vectors encoding the nucleic acid molecules of the present invention, and/or nucleic acid molecules, or other components of the kit of the present invention or may be provided separately in the kit. Particularly, the kit described herein may include one or more additional therapeutic agents such as those described in the “Combined Therapy” described hereabove. The kit(s) may be tailored to a particular cancer for an individual and comprise respective second cancer therapies for the individual as described hereabove.

The instructions related to the use of the multifunctional molecule or pharmaceutical composition described herein generally include information as to dosage, dosing schedule, route of administration for the intended treatment, means for reconstituting the multifunctional molecule and/or means for diluting the multifunctional molecule of the invention.

Instructions supplied in the kits of the invention are typically written instructions on a label or package insert (e.g., a paper sheet included in the kit in the form of a leaflet or instruction manual).

### BRIEF DESCRIPTION OF THE DRAWINGS

FIG. 1: Examples of anti PD-1/Anti CD28 multifunctional molecule format designed for optimal cis-efficacy.

FIG. 2: Anti PD-1*1/anti CD28*1 Format B1 demonstrates efficiently bind to human PD-1 and antagonist activity by blocking PD-L1/PD-1 interaction. (A) PD-1 binding ELISA assay. Human recombinant PD-1 (rPD1) protein was immobilized, and Anti PD-1*1/anti CD28*1 Format B1 was added at different concentrations. Revelation was performed with an anti-human Fc antibody coupled to peroxidase. Colorimetry was determined at 450 nm using TMB substrate. (B) PD-1 binding cytometry assay. Anti PD-1*1/anti CD28*1 Format B1 was incubated with CHO transduced with human PD-1 at 100 nM. Revelation was performed with an PE-labelled anti-human Fc antibody. Binding was measured by cytometry by measurement of MFI (mean fluorescent Intensity) of PE signal (C) Antagonist PD-1/PD-L1 ELISA assay. Human recombinant PD-L1 (rPDL1) protein was immobilized, and Anti PD-1*1/anti CD28*1 Format B1 was mixed at different concentrations with biotinylated human PD-1 at fixed concentration and added to immobilized PD-L1. Revelation was performed with streptavidin coupled to peroxidase. Colorimetry was determined at 450 nm using TMB substrate

FIG. 3: Anti PD-1*1/anti CD28*1 Format B1 efficiently binds to human CD-28. CD28 binding MSD assay. Biotinylated human recombinant CD28 protein was immobilized on streptavidin plate for MSD, and Anti PD-1*1/anti CD28*1 scFv Format B1 was added at different concentrations. Revelation was performed with an anti-human Fc antibody coupled to SulfoTag. Electric signal was determined using MSD system.

FIG. 4: Cis-activity of the Anti PD-1*1/anti CD28 Format B1 specifically reactivates PD-1+CD28+ T cells and spares PD-neg T cells. (A) schematic representation of Cis-activation of anti PD-1/CD28 bispecific antibody. (B) IL-2 secretion bioassay of PD-1+CD28+ or PD-1-CD28+ Jurkat cell after treatment with anti PD-1/anti CD28 multifunctional molecule. Briefly, PD-1+CD28+ Jurkat cell lines were incubated on CD3 coated plate (2.5 ug/mL) in the presence of PD-1*1/anti CD28*1 scFv Format B1 (100 nM), anti PD-1*1, isotype control or anti CD28 (Bivalent anti CD28 positive control of non-specific PD-1 independent activation). IL-2 secretion was quantified in the culture supernatant after 48 hours of stimulation. N=5 independent experiments. IL-2 secretion was normalized to No antibody control condition (C) IL-2 Dose response Cis activation of PD-1+CD28+ Jurkat Tcells treated with Anti PD-1*1 CD28*1 Format B1(●) or negative controls: isotype control (□) or anti PD-1*1 (◯). One representative experiment of 5 independent experiments

FIG. 5: Absence of trans-activity of the Anti PD-1*1/anti CD28 Format B1 on PD-1 negative CD28+ T cells. (A) schematic representation of the experimental settings, PD-1 recombinant protein was coated and PD-1− CD28+ jurkat T cells were incubated with 100 nM of PD-1*1/anti CD28*1 scFv Format B1 (100 nM), anti PD-1*1, isotype control or anti CD28 (Bivalent anti CD28 positive control of non-specific PD-1 independent activation). IL-2 secretion was quantified in the supernatant after 48 of culture using ELISA assay as illustrated in (B).

FIG. 6: Multiple formats of anti-PD-1/AntiCD28 specifically activate PD1+CD28+. (A) PD-1 binding ELISA assay. Human recombinant PD-1 protein was immobilized, and anti-PD-1/anti-CD28 multifunctional molecules Format A1, B1, B2, C1 or C2 were added at different concentrations. Revelation was performed using an anti-human Fc antibody coupled to peroxidase. Colorimetry was determined at 450 nm using TMB substrate. Data were normalized by the condition anti-PD-1*2 or anti-PD-1*1 at 20 nM; considered as 100% of binding for bivalent and monovalent figure respectively. N=2 independent experiments per condition. (B) Antagonist PD-1/PDL1 ELISA assay. Human recombinant PD-L1 protein was immobilized, and anti-PD-1/anti-CD28 multifunctional molecules Format A1, B1, B3, C1, C2, C5 were mixed at different concentrations with biotinylated human PD-1 at a fixed concentration and added to PD-L1 immobilized onto ELISA plates. Revelation was performed with a streptavidin coupled to peroxidase. Colorimetry was determined at 450 nm using TMB substrate. Percentage of antagonist activity was determined by the formula 100-((DO450 nM*100)/DO450 nM No Ab). N=2 independent experiments. (C) CD28 binding MSD assay. Biotinylated human recombinant CD28 protein was immobilized on streptavidin coated plate and anti-PD-1*1/anti-CD28*1 multifunctional molecules Format A1, B1 or B2 were added at different concentrations. Bivalent anti-CD28 binding domain and monovalent anti-PD-1 binding domain were loaded as positive and negative controls, respectively. Revelation was performed with an anti-human Fc antibody coupled to SulfoTag. Electric signal was determined using MSD system. (D) Cis-activity Jurkat T cell-based bioassay. IL-2 secretion bioassay of PD-1+CD28+ or PD-1-CD28+ Jurkat cell after treatment with anti-PD1/anti-CD28 multifunctional molecule. Briefly, PD-1+CD28+ Jurkat cell lines were incubated on CD3 coated plate (2.5-3 μg/mL) in the presence of PD-1/anti-CD28 multifunctional molecules Format A1, B1, B2, B3, B4, C1, C2, C5 and C6 (100 nM), anti-PD-1 or anti-CD28 antibodies (Bivalent anti-CD28 positive control of non-specific PD-1 independent activation). IL-2 secretion was quantified in the culture supernatant after 48 hours of stimulation and data were normalized by the condition without antibody. N=2-7 independent experiments. IL-2 secretion was normalized to no antibody control condition. Statistical analysis was performed using the Mann-Whitney test: ** indicates p-value≤0.001 and ns indicates non-significant. (E) Cis-activity Jurkat T cell-based bioassay with anti-CD28 pre-blocking. IL-2 secretion bioassay of PD-1+CD28+ Jurkat cell after treatment with anti-CD28 antagonist and anti-PD1/anti-CD28 multifunctional molecule. Briefly, PD-1+CD28+ Jurkat cell lines were treated 20 minutes with 1 μg/mL anti-CD28 antagonist, then incubated on CD3 coated plate (3 μg/mL) in the presence of Format B1 PD-1*1/anti-CD28*1 scFv clone 1 (100 nM). IL-2 secretion was quantified in the culture supernatant after 48 hours of stimulation and data were normalized by the condition without antibody. N=3 independent experiments and 2 additional experiments for conditions without anti-CD28 antagonist. Statistical analysis was performed using the Mann-Whitney test: * indicates p-value≤0.05. (F) Trans-activity Jurkat T cell-based assay. PD-1 recombinant protein was coated and PD-1− CD28+ Jurkat T cells were incubated with 100 nM of PD-1/anti-CD28 multifunctional molecules Format A1, B1, B2, B4, C1, C2 and C6 (100 nM), anti-PD-1 or anti-CD28 antibodies (Bivalent anti-CD28 positive control of nonspecific PD-1 independent activation). IL-2 secretion was quantified in the supernatant after 48 hours of culture using ELISA assay and data were normalized by the condition without antibody. N=1-2 independent experiments.

FIG. 7: Other anti-CD28 clones from prior art do not induces cis-activation of PD-1+CD28+ T cells but activate CD28+PD1 negative T cells in trans-manner (A) Schematic representation of Format B1 anti-PD-1*1/anti-CD28*1 scFv antibody (B) Cis-activity Jurkat T cell-based bioassay. IL-2 secretion bioassay of PD-1+CD28+ or PD-1-CD28+ Jurkat cell after treatment with anti-PD1/anti-CD28 multifunctional molecule. Briefly, PD-1+CD28+ Jurkat cell lines were incubated on CD3 coated plate (2.5-3 μg/mL) in the presence of Format B1 anti-PD-1*1/anti-CD28*1 scFv clone 1, clone 2 or clone 3 (100 nM). IL-2 secretion was quantified in the culture supernatant after 48 hours of stimulation and data were normalized by the condition without antibody. N=2-6 independent experiments. (C) Trans-activity Jurkat T cell-based assay. Anti-CD3 and PD-1 recombinant proteins were coated and PD-1-CD28+ Jurkat T cells were incubated with 100 nM of Format B1 anti-PD-1*1/anti-CD28*1 scFv clone 1, clone 2 or clone 3 (100 nM). IL-2 secretion was quantified in the supernatant after 48 hour of culture using ELISA assay and data were normalized by the condition without antibody. N=1-2 independent experiments. (D) Cis-activity Jurkat T cell-based bioassay. IL-2 secretion bioassay of PD-1+CD28+ or PD-1-CD28+ Jurkat cell after treatment with anti-PD1/anti-CD28 unmutated or mutated clone 1. Briefly, PD-1+CD28+ Jurkat cell lines were incubated on CD3 coated plate (2.5-3 μg/mL) in the presence of Format B1 anti-PD-1*1/anti-CD28*1 scFv clone 1 or mutated sequence mutVH_FR1, mutVH_FR2, mutVH_FR3, mutVL_FR1, mutVL_F2, mutVH_CDR1, mutVL_CDR1, mutVL_CDR2 (300 nM). IL-2 secretion was quantified in culture supernatant after 48 hours of stimulation and data were normalized by the condition without antibody and foldchange of PD1+ and PD-1− cells normalized IL-2 secretion were done. N=2 independent experiments.

FIG. 8: anti-CD28/anti-PD-1 constructed with other anti-PD-1 clone and anti-CD28 clone 1 allows cis activation of PD-1+CD28+ T cells. Cis-activity Jurkat T cell-based bioassay. IL-2 secretion bioassay of PD-1+CD28+ or PD-1-CD28+ Jurkat cell after treatment with anti-PD1/anti-CD28 multifunctional molecules. Briefly, PD-1+CD28+ Jurkat cell lines were incubated on CD3 coated plate (3 μg/mL) in the presence of anti-PD-1*1/anti-CD28*1 scFv multifunctional molecules Format B1 with monovalent anti-PD-1 clone 1 sequence or monovalent Pembrolizumab sequence (100 nM), monovalent anti-PD-1 (clone 1 sequence) or anti-CD28 antibodies (bivalent anti-CD28 positive control of nonspecific PD-1 independent activation). IL-2 secretion was quantified in the culture supernatant after 48 hours of stimulation and data were normalized by the condition without antibody. N=4 independent experiments.

FIG. 9: Format B1 anti-PD-1*1/anti-CD28*1 scFv reinvigorates human tumor-infiltrating lymphocytes. IFNy secretion by TILS. Freshly isolated lung tumors (n=2) et ovarian ascites (n=1) were processed to isolate and culture tumoroids. Tumoroids were cultured in triplicate during 48 h in presence of 70 nM of isotype control, anti-PD-1*2 or anti-PD-1*1/anti-CD28*1 scFv (multifunctional molecule Format B1 clone 1) at 37° C. 5% C02. After incubation, supernatants were collected and IFNy secreted by TILS were quantified by MSD or ELISA. Statistical analysis was performed using the Wilcoxon test: * indicates p-value≤0.05.

FIG. 10: Multiple formats of anti-PD-1/anti-CD28 multifunctional molecule have a good pharmacokinetic in mice. C57BL/6Jrj mice received an intravenous single dose with 4 mg/kg or 5 mg/kg of anti-PD-1/anti-CD28 clone 1 multifunctional molecule (Format A1, B1, B3, C2 and C6) or bivalent anti-PD-1 respectively. Blood was sampled at 0.5, 1, 4, 24, 48, 100 and 144 hours. Antibodies concentrations were dosed by ELISA in sera using a mouse anti-human kappa IgG antibody and revelation was performed with a donkey anti-human IgG-peroxidase and TMB substrate.

### EXAMPLES

Further aspects and advantages of the present invention will be described in the following examples, which should be regarded as illustrative and not limiting.

### Material and Methods

### Multifunctional Molecules

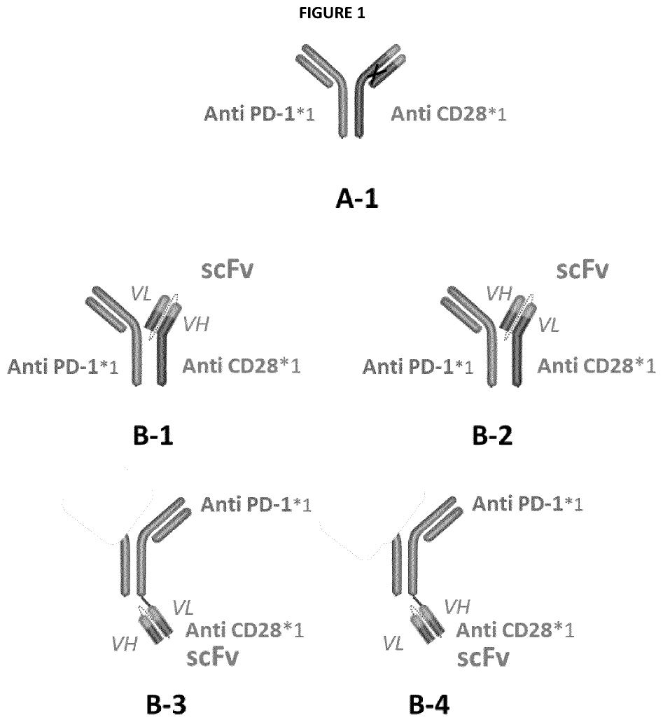

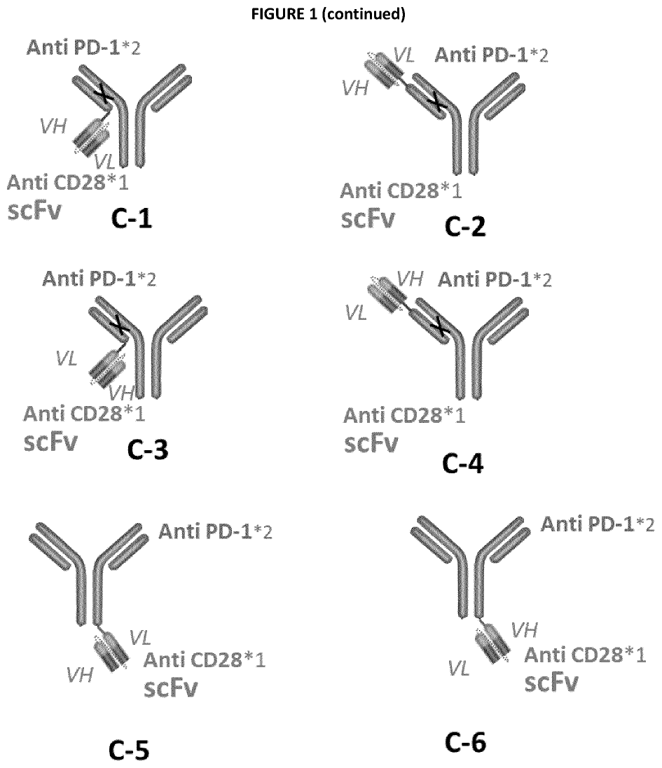

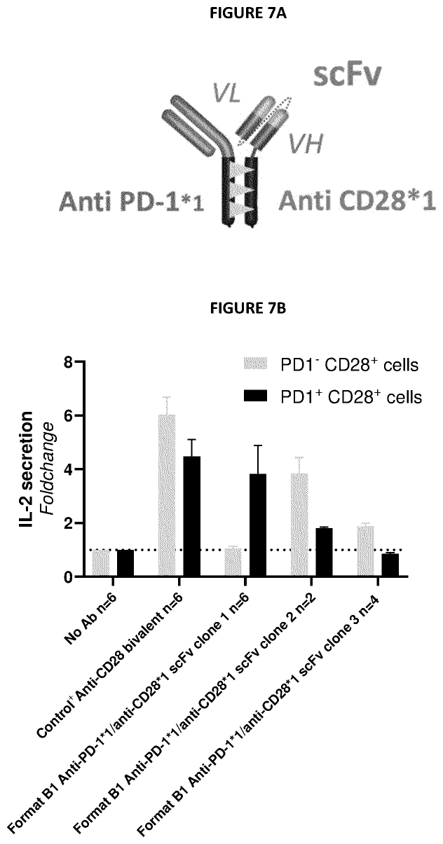

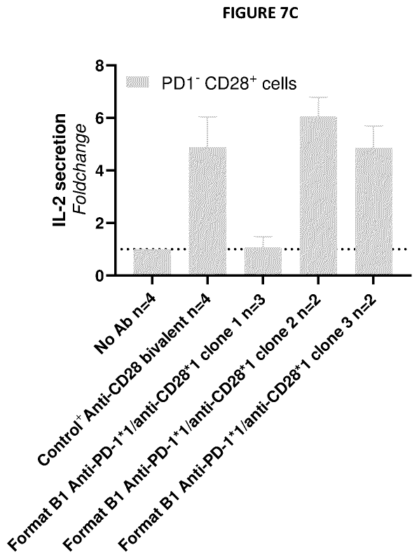

Examples of structures of the multifunctional molecules according to the invention are provided in FIG. 1 and FIG. 7A.

Anti-PD-1 clone 1 is derived from OSE-279 antibody disclosed in patent application WO2020/127366. Such anti-PD-1 binding domain comprises a variable heavy chain such as described under SEQ ID NO: 15 and a variable light chain such as described under SEQ ID NO: 16.

Pembrolizumab is disclosed in patent application WO2008156712. The anti-PD-1 binding domain derived from Pembrolizumab comprises a variable heavy chain such as described under SEQ ID NO: 79 and a variable light chain such as described under SEQ ID NO: 80.

Anti-CD28 clone 1 particularly comprises a variable heavy chain such as described under SEQ ID NO: 44 and a variable light chain such as described under SEQ ID NO: 45.

Anti-CD28 clone 2 is derived from patent application WO2006050949. A multifunctional molecule comprising anti-CD28 clone 2 is particularly described under i) SEQ ID NO: 81 (anti-CD28 arm) and ii) SEQ ID NO: 25 and SEQ ID NO: 16 (anti-PD-1 arm). Such multifunctional molecule is in the B1 format.

Anti-CD28 clone 3 is derived from patent application WO2021260064. A multifunctional molecule comprising anti-CD28 clone 3 is particularly described under i) SEQ ID NO: 82 (anti-CD28 arm) and ii) SEQ ID NO: 25 and SEQ ID NO: 16 (anti-PD-1 arm). Such multifunctional molecule is in the B1 format.

### Mutants of Anti-CD28 Clone 1

Mutants of anti-CD28 clone 1 have been generated. Mutated amino acids are in bold and underlined in the mutant's sequences provided in Table 2 below in comparison to the unmutated sequences. For each mutant, if the VH sequence comprises mutations (e.g., such as described in SEQ ID NO: 83, 84, 85 or 88), the VL sequence is unmutated (i.e., such as described in SEQ ID NO: 45). Conversely, if the VL sequence comprises mutations (e.g., such as described in SEQ ID NO: 86, 87, 89 or 90), the VH sequence is unmutated (i.e., such as described in SEQ ID NO: 44).

| Sequence ID | Name | SEQ ID NO |
|---|---|---|
| QVKLQESGPGLVAPSQSLSITCTVSGFSLTSYGVYWVRQPPGK | scFV anti-CD28 Clone 1 | 44 |
| GLEWLGVIWAGGSTNYNSALMSRLSISKDNSKSQVFLKMNSL | VH (unmutated) | |
| QTDDTAMYYCARDHYGSYGDYYAMDYWGQGTTVTVSS | | |
| DIVLTQSPASLAVSLGQRATISCRASESVEYYDTSLMQWYQQKP | scFV anti-CD28 Clone 1 | 45 |
| GQPPKLLIYAASSVESGVPARFSGSGSGTDFSLNIHPVEEDDIA | VL (unmutated) | |
| MYFCQQSRKVPWTFGGGTKLEIK | | |
| QVTLKESGPGLVKPTETLTITCTVSGFSLTSYGVYWVRQPPGKA | scFV anti-CD28 Clone 1 | 83 |
| LEWLGVIWAGGSTNYNSALMSRLTISKDNSKSQVFLTMTSMD | mutVH_FRs1 | |
| PVDTAMYYCARDHYGSYGDYYAMDYWGQGTTVTVSS | | |
| QVQLVESGGGVVQPGRSLRLSCAVSGFSLTSYGVYWVRQAPG | scFV anti-CD28 Clone 1 | 84 |
| KGLEWLGVIWAGGSTNYNSALMSRLTISKDNSKSTVFLQMTSL | mutVH_FRs2 | |
| RAEDTAMYYCARDHYGSYGDYYAMDYWGQGTLVTVSS | | |
| QVQLQESGPGLVKPSETLSITCTVSGFSLTSYGVYWVRQPPGK | scFV anti-CD28 Clone 1 | 85 |
| GLEWLGVIWAGGSTNYNSALMSRLTISKDNSKSQVFLKMSSVT | mutVH_FRs3 | |
| AADTAMYYCARDHYGSYGDYYAMDYWGQGTLVTVSS | | |
| DIQLTQSPSSLSASVGDRVTISCRASESVEYYDTSLMQWYQQK | scFV anti-CD28 Clone 1 | 86 |
| PGKAPKLLIYAASSVESGVPSRFSGSGSGTDFSLTISSLQPEDIA | mutVL_FRs1 | |
| MYFCQQSRKVPWTFGGGTKVEIK | | |
| DIVLTQSPDSLAVSLGERATISCRASESVEYYDTSLMQWYQQKP | scFV anti-CD28 Clone 1 | 87 |
| GQPPKLLIYAASSVESGVPDRFSGSGSGTDFSLTISSVQAEDVA | mutVL_FRs2 | |
| MYFCQQSRKVPWTFGGGTKVEIK | | |
| QVKLQESGPGLVAPSQSLSITCTVSGFSLSSYGVYWVRQPPGK | scFV anti-CD28 Clone 1 | 88 |
| GLEWLGVIWAGGSTNYNSALMSRLSISKDNSKSQVFLKMNSL | mutVH_CDRs1 | |
| QTDDTAMYYCARDHYGSYGDYYAMDYWGQGTTVTVSS | | |
| DIVLTQSPASLAVSLGQRATISCRASQSVEYYDTSLMQWYQQK | scFV anti-CD28 Clone 1 | 89 |
| PGQPPKLLIYAASSVESGVPARFSGSGSGTDFSLNIHPVEEDDIA | mutVL_CDRs1 | |
| MYFCQQSRKVPWTFGGGTKLEIK | | |
| DIVLTQSPASLAVSLGQRATISCRASESVEYYDTSLMQWYQQKP | scFV anti-CD28 Clone 1 | 90 |
| GQPPKLLIYAASSRESGVPARFSGSGSGTDFSLNIHPVEEDDIA | mutVL_CDRs2 | |
| MYFCQQSRKVPWTFGGGTKLEIK | | |

Table 2. hCD28 binding of anti-CD28/anti-PD-1 multifunctional molecule (Format B1) and anti-CD28 bivalent using Biacore Technology. Different constructions of Format B1 anti-PD-1*1/anti-CD28*1 scFV were generated with anti-CD28 scFv clone 1 (unmutated), 3 different framework regions in VH domain (mutVH_FRs1, mutVH_FRs2, mutVH_FRs3), 2 different framework regions in VL domain (mutVL_FRs1, mutVL_FRs2), 1 CDR mutations in VH domain (mutVH_CDRs1) or 2 CDR mutations in VL domains (mutVL_CDRs1, mutVL_CDRs2).

In particular, when the mutations are in the framework regions of the anti-CD28 binding domain, mutVH_FRs1 comprises amino acid substitutions in the heavy chain framework region HFR1, HFR2 and HFR3; mutVH_FRs2 comprises amino acid substitutions in HFR1, HFR2, HFR3 and HFR4; mutVH_FRs3 comprises amino acid substitutions in HFR1, HFR3 and HFR4; mutVL_FRs1 comprises amino acid substitutions in the light chain framework regions LFR1, LFR2, LFR3 and LFR4; mutVL_FRs2 comprises amino acid substitutions in LFR1, LFR3 and LFR4, when compared to the anti-CD28 Clone 1 original sequences.

When the mutations are in the CDRs of the anti-CD28 binding domain, mutVH_CDRs1 comprises an amino acid substitution in the heavy chain CDR1 (the sequence of HCDR1 being determined according to the IGMT method); mutVL_CDRs1 comprises an amino acid substitution in the light chain CDR1 (the sequence of LCDR1 being determined according to the IGMT or Kabat method); mutVL_CDRs2 comprises an amino acid substitution in the light chain CDR2 (the sequence of LCDR1 being determined according to the IGMT or Kabat method), when compared to the anti-CD28 Clone 1 original sequences.

### PD-1 ELISA Binding

Recombinant hPD1 (Sino Biologicals, Beijing, China; reference 10377-H08H) was immobilized on 96well. Plate at 0.5 μg/ml in carbonate buffer (pH9.2) and purified antibodies were added to measure binding. After incubation and washing, peroxidase-labeled donkey anti-human IgG (Jackson Immunoresearch; USA; reference 709-035-149) was added and revealed by conventional methods.

### Binding Analysis to PD1+ Expressing CHO Cells

CHO was transduced with human PD1 gene and selected to express PD1 receptor. Purified antibodies were added at 100 nM on CHO PD1+. After incubation and washing, Phycoerythrin-labeled anti-human IgG (Biolegend; reference 409303) was added and revealed by conventional methods on cytometry.

### PD-1/PD-L1 Antagonist ELISA Assay

Competitive ELISA assay was performed by PD-1: PD-L1 Inhibitor Screening ELISA Assay Pair (AcroBiosystems; USA; reference EP-101). In this assay, recombinant human PDL1 was immobilized on plastic at 2 μg/ml in PBS pH7.4 buffer. Purified antibodies (at different concentrations) were mixed with 0.66 μg/ml final (fix concentration) of biotinylated Human PD1 (AcroBiosystems; USA; reference EP-101) to measure competitive binding for 2 h at 37° C. After incubation and washing, peroxidase-labeled streptavidin (Vector laboratoring; USA; reference SA-5004) was added to detect Biotin-PD-1Fc binding and revealed by conventional methods.

### CD28 Binding Measurement Using MSD Technology

For activity ELISA assay, recombinant human CD28 (bio-techne; reference 342-CD-200) was immobilized on plastic at 5 μg/ml in PBS 1× (pH7.4) and purified antibodies and multifunctional molecules were added to measure binding. After incubation and washing, sulfoTag-labeled monoclonal mouse anti-human kappa antibody (clone NaM76-5F3, labeled with MSD GOLD SULFO-TAG NHS-Ester reagent (MSD; reference #R91AO-1)) was added and revealed by MSD system.

### Jurkat Phenotyping

Jurkat T cell lymphoma were phenotyping using flow cytometry. Jurkat were incubated with 1× yellow live dead (#L34968 Thermofisher) during 20 minutes on ice. Cells were wash three times in PBS1×2 mM EDTA 2% FBS. Jurkat were incubated with anti-CD28-FITC (1/20; #555728 BD), during 30 minutes on ice. Cells were wash three times in PBS1×2 mM EDTA 2% FBS. Jurkats were analyzed on classical flow cytometer Cytoflex (Beckman). CD28 expression were studied in viable (LIVE DEAD negative) single events using unstained and/or FMO (fluorescent minus one) controls.

### Cis Activity Bioassay

Jurkat T cell lymphoma expressing CD28 were transduced with human PD-1 allowing a stable expression of human PD-1 on cell surface. For bioassay, Jurkat CD28+PD-1− or Jurkat CD28+PD-1+ were cultivated on CD3 coated plate (2.5-3 ug/mL OKT3) in the presence of anti-PD-1/anti-CD28 multifunctional molecules. After 48 hours of incubation, IL-2 secreted by Jurkat cells was quantified in the supernatant using ELISA (Duoset R&D kit #DY202-05 or OptEIA™ BD Kit #555190).

### Trans Activity Bioassay

Jurkat CD28+PD-1− cells were cultivated on CD3 (2.5-3 ug/mL OKT3)+human PD-1 coated plate in the presence of anti-PD-1/anti-CD28 multifunctional molecules. After 48 hours of incubation, IL-2 secreted by Jurkat cells was quantified in the supernatant using ELISA (Duoset R&D kit #DY202-05 or OptEIA™ BD Kit #555190).

### Biacore

hCD28 recombinant protein (bio-techne, reference 342-CD-200) was immobilized to the CM5 sensorship at 20 ug/mL in Na Acetate pH5 in the Biacore T200. Binding was measured by flowing anti-CD28 bivalent antibody or anti-CD28/anti-PD-1 (Format B1) antibody in HBS EP buffer (provided by CYTIVA) at escalating doses (0, 187, 375, 750, 1500 and 3000 nM) at a flow rate of 40 uL/min. The antigen-antibody association kinetics was followed for 2 minutes, and the dissociation kinetics was followed for 10 minutes. The association and dissociation curves were fit to a bivalent model.

### Pharmacokinetics in Mice

A single dose of anti-PD-1/anti-CD28 (4 mg/kg) or anti-PD-1*2 (5 mg/kg) were intravenously injected in female 9 weeks old C57BL6RJ wild-type mice. Following administration, blood was collected at multiple time point (0.5, 1, 4, 24, 48, 72, 100 and 144 hours), diluted 50× in PBS, centrifugated and supernatant (sera) containing antibodies were stored at −20° C. (short-term) or −80° C. (long-term) until quantification analysis.

Drug concentration in the sera was determined by ELISA using an immobilized mouse anti-human kappa IgG (clone NaM76-5F3) diluted at 1 μg/ml in borate buffer 1×. Detection was performed with a peroxidase-labeled donkey anti-human IgG (Jackson Immunoresearch; USA; reference 709-035-149) and revealed by conventional methods.

### TILS-IFNy Secretion Quantification in Human Tumoroid

Tumors were freshly collected after resection surgery and stored at 2-8° C. in MACS tissue storage (Miltenyi) until processing (24 h after surgery maximal). Two sample of lung cancer were collected for this experiment. Ovarian ascites was freshly collected after punction; one ovarian ascites was collected for this experiment. Sample was collected from patient who received chemotherapy or radiotherapy treatment, none of them were treated with Immune Checkpoint blockade therapy.

Tumors were minced in 50% RPMI 1640 (Gibco) 50% TexMacs (Miltenyi) supplemented with 5% FBS 100 U/mL penicillin and 0.1 mg/mL Streptomycin/Primocin in fragment of 2-3 mm³. Fragments were plated homogenously in Ultra Low Attachment plate. 70 nM of antibodies (Isotype control, anti-PD-1*2 and anti-PD-1*1/anti-CD28*1 scFv format B1 clone 1) were added to tumoroids (triplicate) and plates were incubated at 37° C. 5% C02. After 48 h of incubation, culture supernatants were collected and stored at −80° C. until MSD IFNy quantification.

Ascites was centrifuged, pellets was washed with PBS 5 mM EDTA. After a second centrifugation, red-blood cells were depleted with RBC Lysis Buffer. Remaining cells were resuspended in 50% RPMI 1640 (Gibco) 50% TexMacs (Miltenyi) supplemented with 5% FBS 100 U/mL penicillin and 0.1 mg/mL Streptomycin/Primocin and plated in Ultra Low Attachment plate. 70 nM of antibodies (Isotype control, anti-PD-1*2 and anti-PD-1*1/AntiCD28*1 scFv format B1 clone 1) were added to cells (triplicate) and plates were incubated at 37° C. 5% C02. After 48 h, culture supernatants were collected and stored at −80° C. until MSD quantification. IFNy secreted by TILS in tumoroids were quantified by S-plex Human IFN-γ kit (#K151X9S-2 MSD).

### Results

### Example 1. Anti PD-1/AntiCD28 Multifunctional Molecule Efficiently Constructed with a Monovalent Anti PD-1 Efficiently Binds and Antagonizes Human PD-1 Inhibitory Signaling

Binding of the anti PD-1/anti CD28 (Format B1) to human PD-1 receptor was assessed by ELISA. Briefly, recombinant human PD-1 receptor was coated on the plate, multifunctional molecules were added at serial dilutions, then revealed with a secondary anti human IgG antibody coupled to peroxidase +TMB. Binding to PD-1 expressing cells was also evaluated after incubation of human PD-1 transduced CHO cells with anti PD-1/anti CD28+an anti-human IgG-PE antibody. Antagonist activity of the multifunctional molecules against PD-1 were assessed by competitive ELISA assay (Acrobiosystem).

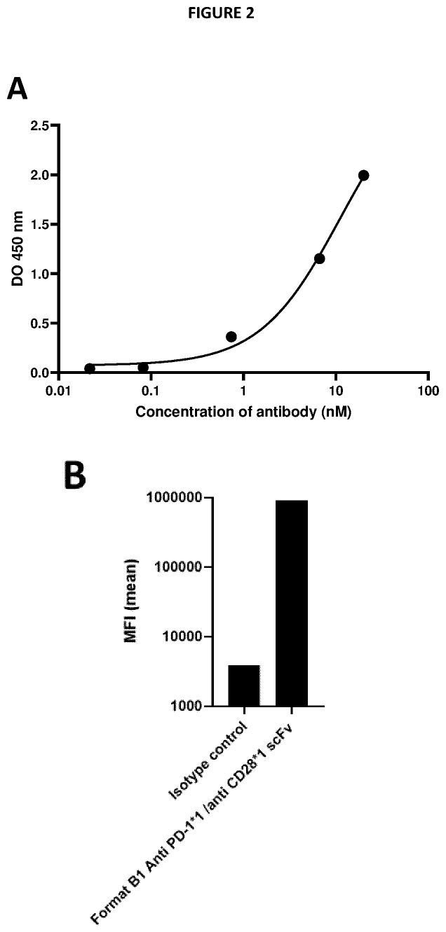

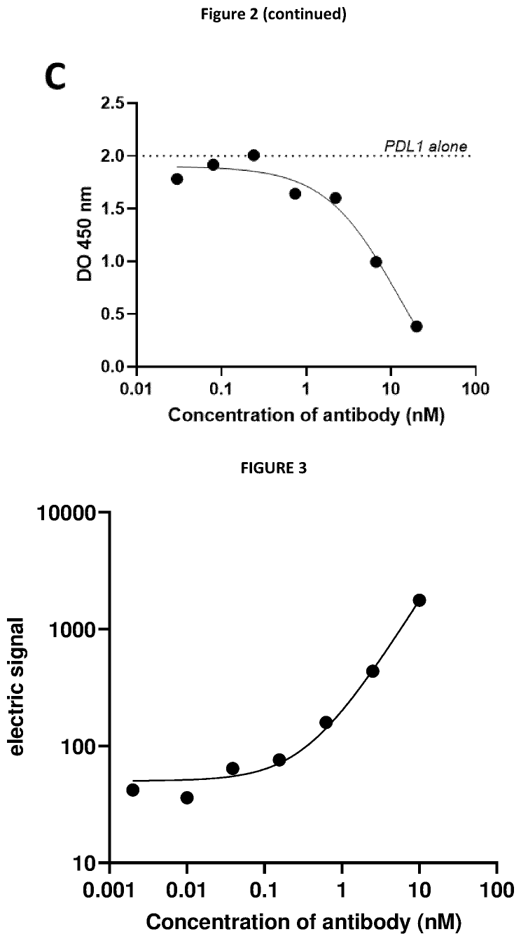

As shown in FIG. 2, format B1 constructed with a monovalent anti PD-1 binds to human PD-1 receptor, and efficiently antagonizes PD-L1 interaction, demonstrating that the anti-PD-1 domain of the multifunctional molecule even in monovalent format can target PD-1+ exhausted T cells and block PD-L1/PD-1 engagement to consequently antagonize PD-1 mediated inhibitory signaling and promote reinvigoration of PD-1+ exhausted T cell reinvigoration (enhance effector functions).

### Example 2. Anti PD-1/AntiCD28 Multifunctional Molecule Efficiently Constructed with a Monovalent Anti CD28 can Bind to Human CD28

FIG. 3 shows that the anti PD-1/anti CD28 (Format B1) binds to human CD28 as assessed by ELISA using MSD technology. Briefly, biotinylated human recombinant CD28 protein was immobilized on streptavidin plate for MSD, and Anti PD-1*1/anti CD28*1 scFv Format B1 was added at different concentrations. Revelation was performed with an anti-human Fc antibody coupled to SulfoTag.

Electric signal was quantified using MSD system.

### Example 3. Anti PD-1/AntiCD28 scFv multifunctional molecules (Format B1) Cis target and specifically activate PD-1+CD28+ T cells while sparing PD-1 negative CD28+ T cells

Cis efficacy of the anti PD-1/anti CD28 multifunctional molecules were evaluated in cell-based bioassay using Jurkat T cells expressing CD28 receptor only or co-expressing CD28 and PD-1 receptors. After treatment with the anti PD-1/anti CD28 (Format B1) on anti CD3 coated plate (2.5 ug/mL), IL-2 secretion was quantified in the supernatant 48 hours after incubation using ELISA assay.

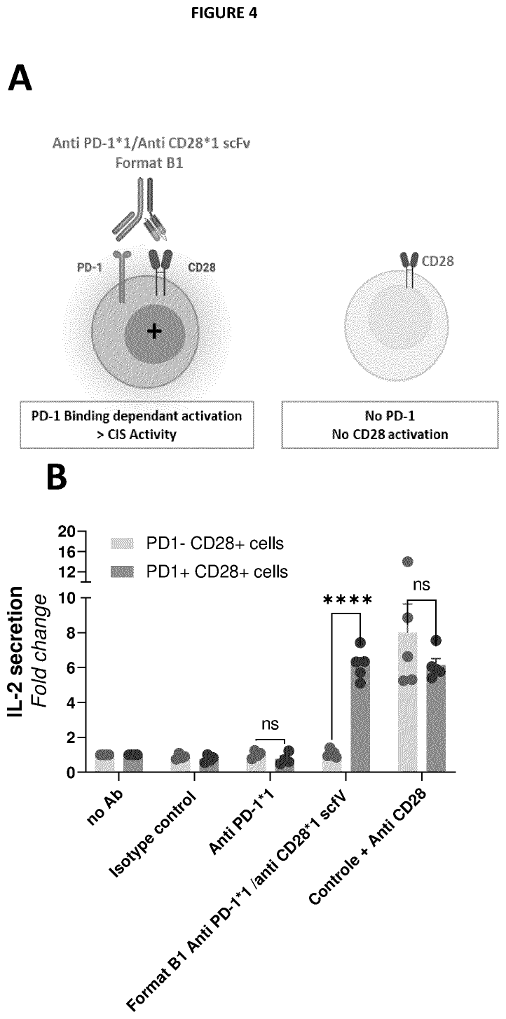

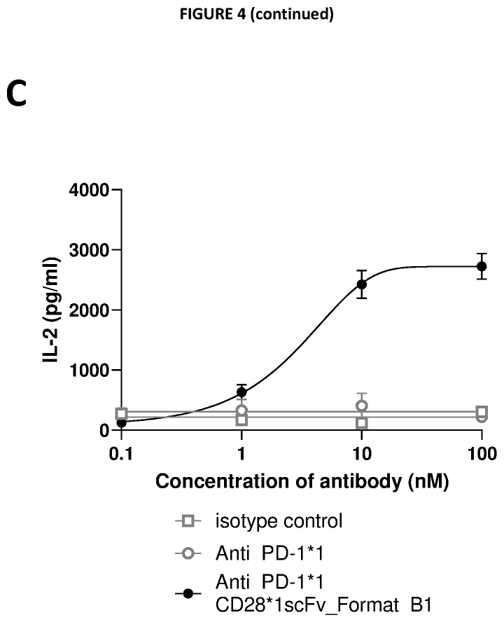

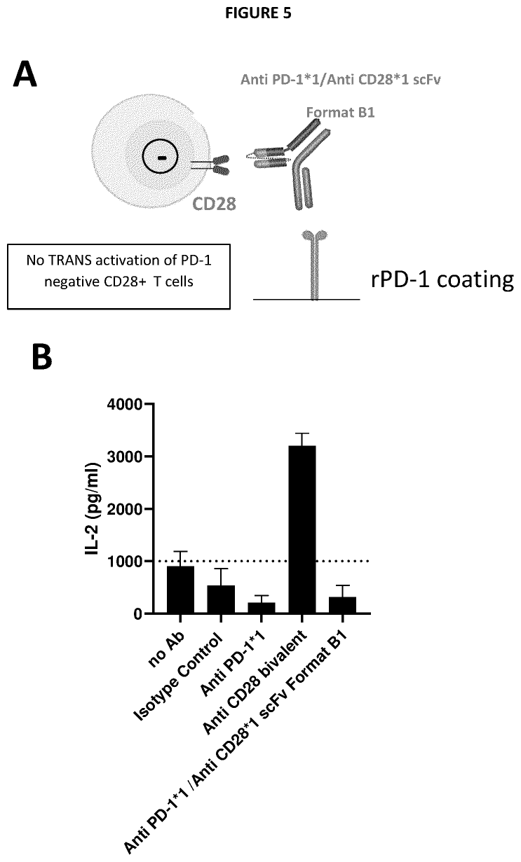

FIG. 4 A, B and C demonstrate that the anti PD-1/anti CD28 (Format B1). Multifunctional molecules can promote T cell activation (IL-2 secretion) in PD-1+CD28+ T cells, while the drug has no effect on PD-1 negative T cells demonstrating the cis specific activity (i.e. on the same cells) of the molecule to trigger the CD28 signaling only into PD-1+ T cells. This effect is dose dependent as demonstrated in Figure C. In parallel, the inventors also shows that anti PD-1 bivalent antibodies do not induce activation of T cells whereas bivalent Anti-CD28 promotes activation in all T cell population irrespectively of PD-1 expression. As CD28 is express on all T cells, a bivalent anti CD28 antibody will trigger activation of all T cells inducing high immunotoxicity and cytokine storm as described in previous clinical trials (Suntharalingam, et al., NEJM 2016). In contrast the anti PD-1/Anti CD28 multifunctional molecule described by the inventors demonstrate selective activation of PD-1+ T cells (tumor specific exhausted T cells) while sparing activation of PD-1 negative T cells (naïve CD28+ non-specific T cells) which will prevent non-specific toxicity induced by bivalent anti CD28 antibodies.

To confirm that the effect induced by the anti PD1/anti CD28 multifunctional molecule is only mediated in cis-manner and that the drug has no trans activity, the inventors studied the effect of the drug on PD-1 coated plate to force the binding of the Anti PD-1 domain of the Bispecific as illustrated on FIG. 5A. Jurkat T cells expressing only CD28 receptor was then added to the plate and IL-2 secretion was dosed by ELISA in the supernatant 48 h after co-incubation FIG. 5B demonstrated that the anti PD-1/anti CD28 (Format B1) multifunctional molecule does not induce secretion of IL-2 when PD-1 is coated on the plate, suggesting that the multifunctional molecule has no trans activity effect on PD-1 negative CD28+ T cells. These data confirm that the anti PD-1/anti CD28 described by the inventors will not activate PD-1 negative cells (naïve T cells) and has only a cis-dependent properties by triggering CD28 signal into the same cells expressing PD-1 receptor.

Altogether, the Anti PD-1/Anti CD28 multifunctional molecules described by the inventors will act in cis-dependent manner, by triggering the CD28 costimulatory signal but also by blocking PD-1 inhibitory signaling to reinvigorate PD-1+CD28+ exhausted T cell effector functions without activating non-specific T cells. On the opposite bivalent anti-CD28 antibodies or other anti CD28 bispecific antibodies described by others, having a monovalent CD28 and a monovalent anti-TAA domain will activate in trans all T cells PD-1 tumor specific T cells as well as PD-1 negative non-specific T cells.

### Example 4. Multiple Formats of Anti-PD-1/Anti-CD28 Multifunctional Molecule Efficiently and Specifically Activate PD1+ CD28+ Cells in Cis-Manner while Sparing PD1− CD28+ Cells

Other formats of the anti-PD-1/anti-CD28 multifunctional than described in the Example 2 were constructed using a bivalent anti-PD-1 antibody fused to an anti-CD28 scFv (formats C1 and C2), a monovalent anti-PD-1 antibody fused to an anti-CD28 scFV (formats B1 and B2) or an monovalent anti-PD-1 Fab fused to an monovalent anti-CD28 Fab CrossMAb (Format A1) as described in FIG. 1. These different constructions were generated with the anti-CD28 clone 1. Antibodies capacity to cis-activate PD-1+CD28+ T cells was evaluated using PD1+CD28+ Jurkat T cells bioassay. Antibodies capacity to bind to human CD28 (MSD technology) or to human PD-1 (ELISA assay) was studied as detailed in the Examples 2 and 3. Binding of anti-PD-1/anti-CD28 multifunctional molecules to human PD-1 were assessed by ELISA after coating of human PD-1 recombinant protein on ELISA plate.

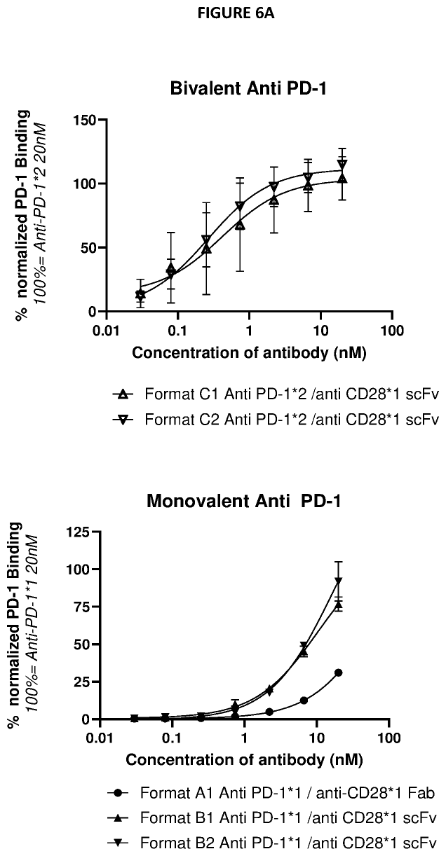

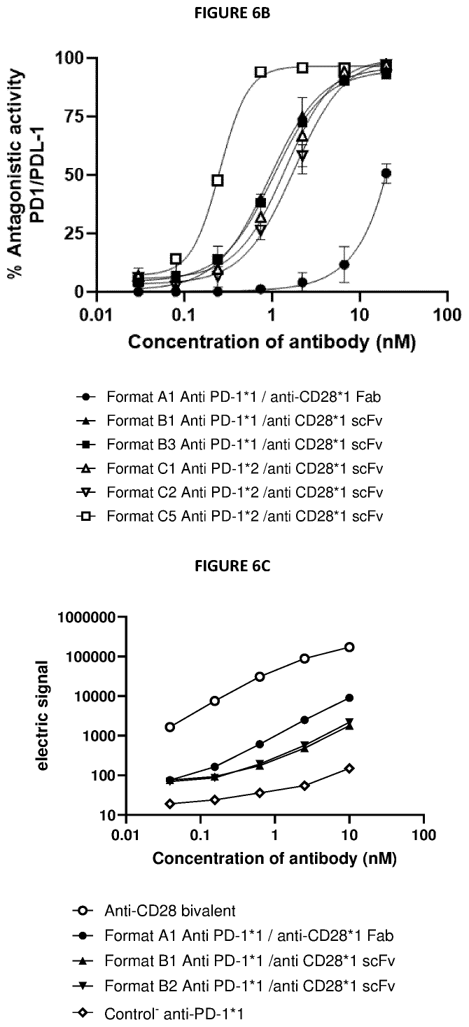

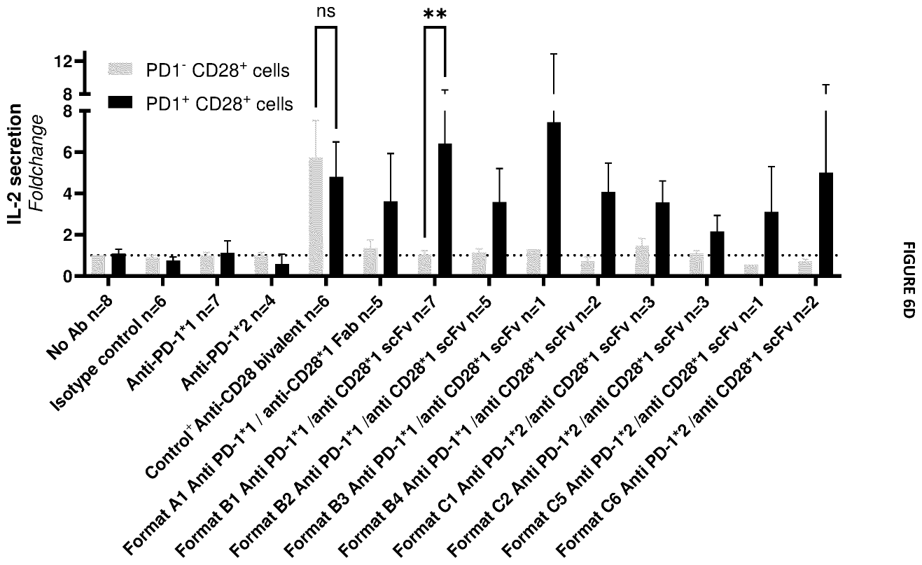

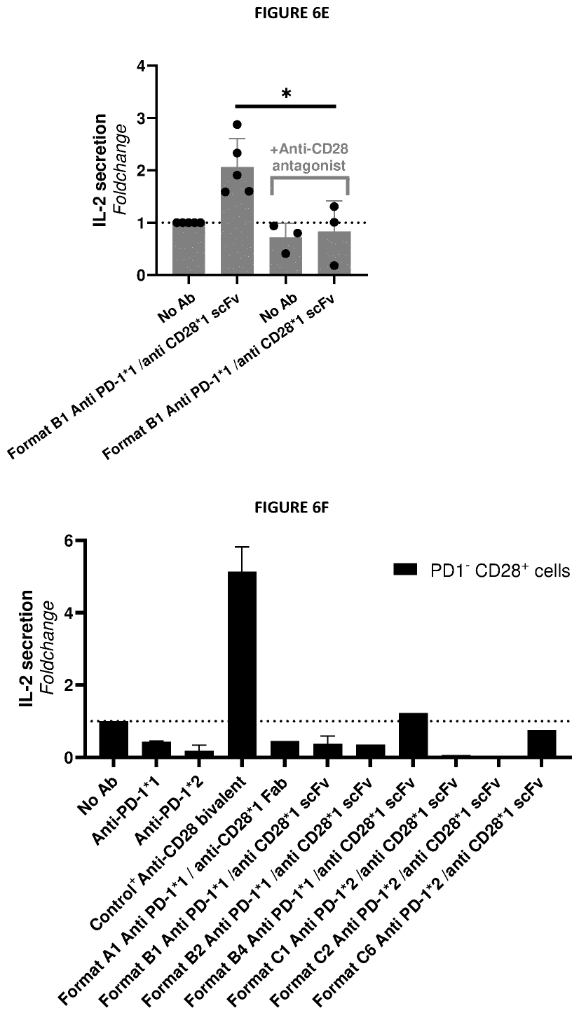

FIG. 6A demonstrates that the different formats of bispecific antibodies with bivalent (Format C1 and C2) or monovalent (Format B1 and B2) anti-PD-1 domains efficiently bind to human PD1 receptor. The antibody (Format A1) that uses CrossMAb technology for the anti-CD28 domain binds to PD-1 but with lower potency. The fusion of scFv anti-CD28 domain to the C-Terminal or N-Terminal domain of the anti-PD-1 binding domain does not modify the capacity to bind PD-1 receptor since similar binding activity is observed between Format C1 and C2. Antagonist activity of anti-PD-1/anti-CD28 multifunctional molecules on PD-L1/PD-1 axis was studied using a competitive ELISA assay (Accrobiosystems) in comparison with two other formats scFv anti-CD28 fused to C-terminal of bivalent anti-PD1 (format C5) or monovalent anti-PD1 (format B3).

FIG. 6B demonstrates that all formats have an effective antagonist anti-PD-1 activity. Format C5 demonstrated a high antagonist activity and anti-PD-1 monovalent formats (Bi and B3) showed similar antagonist activity to bivalent anti-PD-1 construction (C1 and C2).

Next, the inventors assessed the property of the different constructions to bind to human CD28. Anti-PD-1*1 construction was used as negative control sample for this assay.

FIG. 6C validates that all different formats of bispecific molecules comprising an anti-CD28 scFv format (Formats B1 and B2) or with an anti-CD28 Fab format (Format A1) bind to human CD28. The orientation of VH and VL domains does not affect the binding to human CD28, as B1 and B2 constructions have an equivalent binding capacity. All anti-PD-1/anti-CD28 constructions comprising a monovalent anti-CD28 targeting domain have a lower binding potency to human CD28 than the anti-CD28 bivalent clone 1 antibody with no anti-PD-1 valency (positive control of the experiment) (FIG. 6C and Table 1 below). An affinity equal to 75.4 nM was observed for bivalent anti-CD28 antibody and a KD up to 5000 nM was estimated for the anti-PD-1/anti-CD28 antibody (Format B1). Bivalent anti-CD28 antibodies can induce non-specific T cell activation (independently of TCR triggering) and highly toxic effect in human patients leading to unspecific activation of naïve-T cells and high cytokine release. In order to specifically activate PD-1+ T cells and avoid triggering/clustering of CD28 receptor on naïve T cells independently of PD-1, the inventor designed anti-PD-1/anti-CD28 multifunctional molecule with an anti-CD28 monovalent format having low CD28 binding properties to favor targeting of the multifunctional molecules to PD-1 receptor and strictly reactivate CD28 signaling into PD-1+ tumor specific T cells.

| | ka (1/Ms) | kd (1/s) | KD (M) |
|---|---|---|---|
| anti-CD28 bivalent | 1.12E+04 | 8.42E−04 | 7.5E−8 |
| anti-PD-1/anti-CD28 | 4616 | 0.02492 | 5.4E−6 |
| Format B1 | | | |

Table 1. hCD28 binding of anti-CD28/anti-PD-1 multifunctional molecule (Format B1) and anti-CD28 bivalent using Biacore Technology. hCD28 recombinant protein (bio-techne, reference 342-CD-200) was immobilized to the CM5 sensorship then anti-CD28 bivalent antibody or anti-CD28/anti-PD-1 (Format B1) multifunctional molecule was added at escalating doses and kd and ka were measured.

Using a cell-based bioassay as described in Example 3, the cis-activity of the different constructions was evaluated by quantifying secretion of IL-2 by PD-1+CD28+ cells or by PD-1 negative CD28+ Jurkat cells. FIG. 6D demonstrates that all the tested anti-PD-1/anti-CD28 multifunctional molecules do not activate PD-1 negative CD28+ T cells (Grey bars) while activating PD1+CD28+ T cells (2 to 7-fold IL-2 secretion increased versus a condition without antibodies was observed (Black bars). Bivalent anti-CD28 (positive control) activated both PD1+ and PD1− CD28+ T cell population with similar efficacy, confirming that both PD-1+ and PD1− cell in the bioassay can be activated with anti-CD28 antibodies. Unexpectedly, despite the low affinity/avidity of the anti-CD28 binding domain in a single arm format compared to bivalent anti-CD28 (control+), an equivalent activation of PD-1+CD28+ T cells is observed for some constructions, confirming the high potency of the multifunctional molecules to co-target PD-1 and CD28 on the same T cells.

Bivalent or monovalent anti-PD-1 constructions without anti-CD28 arm (negative control) do not induce IL-2 secretion, demonstrating that CD28 signaling engagement is required to activate PD-1+ cells with multifunctional molecules. Surprisingly, even if the binding of the different anti-PD-1/anti-CD28 multifunctional molecule to PD-1 receptor is reduced with a single-arm anti-PD-1 antibody (monovalent anti-PD-1) compared to other constructions with 2 arms anti-PD-1 antibody (as evidenced on FIG. 6A), all monovalent constructions have similar efficacy than constructions with bivalent anti-PD-1 arm to cis-activate PD-1+CD28+ T cells. Altogether, these data underline that all formats of the anti-PD-1/anti-CD28 multifunctional molecules comprising CD28 clone 1 can promote a strict cis-activation signaling into PD-1+CD28+ while sparing PD-1 negative CD28+ T cells.

To confirm that activation of T cells observed in the bioassay is induced by the engagement of CD28 signaling, the inventors performed the same bioassay with an anti-CD28 blocking antibody preincubated on PD-1+CD28+ cells before incubation of anti-PD-1*1/anti-CD28*1 scFv multifunctional molecule (Format B1). FIG. 6E shows that IL-2 secretion is significantly abrogated in PD1+ cells in the presence of anti-CD28 antagonist treatment compared to no pre-blocking condition. This data suggests that IL-2 secretion induced by anti-PD-1/anti-CD28 multifunctional molecules is dependent on anti-CD28 agonist activity. On the other hand, the anti-PD-1 domain allows the specific PD-1 targeting and the cross-linking of CD28 molecule which consequently triggers CD28 costimulatory cascade signaling pathway into PD-1+ T cells.

Finally, the inventors studied trans-activity of the different anti-PD1/anti-CD28 multifunctional molecules formats on PD-1 coated ELISA plate to force the binding of the anti-PD-1 domain and trans-presentation of anti-CD28 domain, as illustrated on FIG. 5A and described in Example 3. This assay mimics trans-presentation by PD-1+ cells of anti-CD28 domain and allows to evaluate the potential non-specific effect of the molecules to trigger CD28 signaling into PD-1 negative T cells. FIG. 6F demonstrated that no anti-PD-1/anti-CD28 multifunctional molecules formats induce the secretion of IL-2 by PD1− CD28+ cells, even in the condition with human PD-1 coating, confirming that the anti-PD-1/anti-CD28 multifunctional molecule constructed with CD28 clone 1 has no trans activity effect on PD-1− CD28+ T cells, regardless of the design (i.e., format) of the multifunctional molecule.

Altogether, these data confirm that multiple formats of anti-PD-1/anti-CD28 multifunctional molecules comprising anti-CD28 clone 1 will not activate PD-1 negative cells (e.g., naive T cells) but can specifically (1) block PD-1/PD-L1 inhibitory signaling (2) induce CD28 activating-signaling into PD-1+ cells to promote reinvigoration of exhausted PD1+ T cells and anti-tumor efficacy.

### Example 5. Cis-Activity of Anti-PD-1/Anti-CD28 Multifunctional Molecule is Specific to Anti-CD28 Clone 1 Epitope Binding Domain

To evaluate whether the anti-CD28 epitope binding domain is critical for the cis-activity property of the multifunctional molecule, the inventors next constructed additional multifunctional molecules in Format B1 (FIG. 7A) with other anti-CD28 sequences (clone 2 and 3) including the superagonist TGN1412 (clone 2) to trigger CD28 signaling after targeting in trans-manner tumor cell antigen. Agonist properties of the different anti-CD28 clones were first confirmed using human PBMCs incubated on anti-CD3+anti-PD-1/anti-CD3 construction coated plate to induce crosslinking of CD28 with coated ELISA plate) (Data not shown). Then, cis-activity properties of the different anti-PD-1/anti-CD28 molecules (soluble form) were assessed using PD-1+ versus PD-1− T cell-based assay described in Example 3. FIG. 7B demonstrated that Clone 2 induces a high activation of PD-1 negative T cells (4-fold). Anti-PD-1/anti-CD28 clone 3 also induces non-specific activation of PD-1 negative cells (2-fold). Interestingly, anti-PD-1/anti-CD28 clone 2 and clone 3 induces high IL-2 secretion when PD-1 recombinant protein is coated on ELISA plate, suggesting that these molecules can activate non-PD-1 T cells in a trans-manner (FIG. 7C), unlike clone 1. Finally, data demonstrate that construction comprising the anti-CD28 clone 1 sequence has a particular property to allow T cells cis activation while other clones failed to provide such cis-targeting effect.

Different mutations in framework regions (FRs) or complementary determining regions (CDRs) of the anti-CD28 clone 1 were generated (see Material and Method and Table 2). The different anti-CD28 mutants have been included as scFv into multifunctional molecules in the B1 format with a monovalent anti-PD-1 binding domain (Fab). The cis-activity properties of such multifunctional molecules were tested on PD1+ and PD1− Jurkat T cells and FIG. 7D represents the ratio of IL2 secretion between PD1+ and PD1. FIG. 7D shows that all the different constructions (i.e., comprising different anti-CD28 variants) maintain the activation of PD-1+ CD28+ cells and not on PD1− CD28+ cells. Similarly, cis-activity with the mutants of the anti-CD28 clone 1 comprising different Frameworks or CDRs mutations was observed as compared to the anti-PD1/anti-CD28 unmutated clone 1 (used in previous examples) used in this assay as a positive control of cis-activity. Altogether, these data show that the clone 1 cis-activity is maintained for the multifunctional molecule event with mutation(s) in the anti-CD28 scFv domain.

### Example 6. Anti-PD-1/Anti-CD28 Multifunctional Molecule can be Constructed with Different Anti-PD-1 Antibody and Maintain Cis-Activity Property

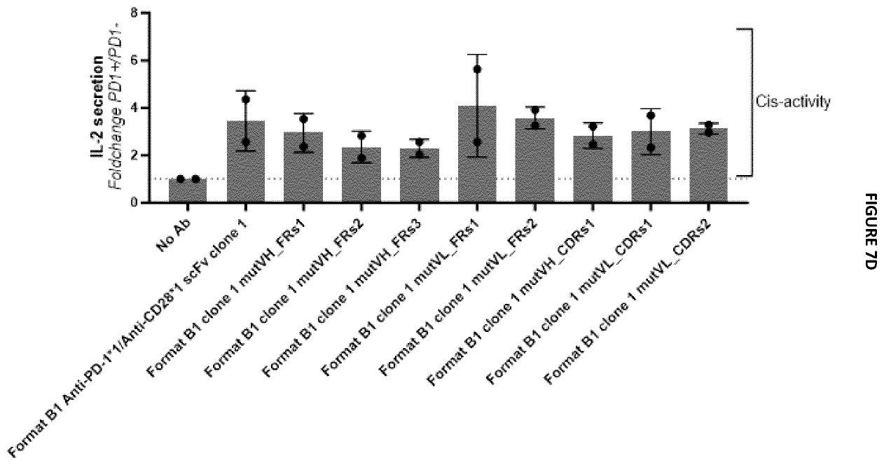

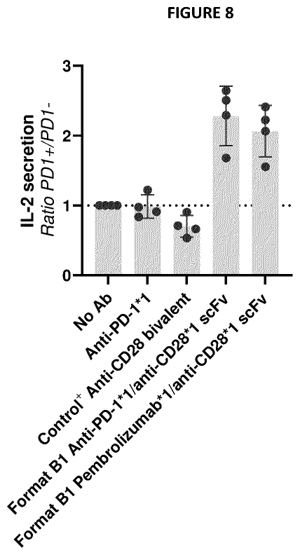

Next, the inventors evaluate whether the anti-PD-1 binding domain impacts the cis-activity capacity of the multifunctional molecule. The cis-activities of two anti-PD-1 clones were assessed: anti-PD-1 clone 1 (as described in Example 1 to 5) and Pembrolizumab (FDA approved anti-PD-1 antibody). Cis-activity of multifunctional molecules in format B1 comprising Pembrolizumab or anti-PD-1 clone 1 fused to anti-CD28 clone 1 were compared in T cell-based assay detailed in Example 3. FIG. 8 demonstrates that both bispecific antibodies have cis-activity properties with a specific targeting of PD-1+ cells and similar ratio of targeting PD-1+ versus PD-1 negative cells. This confirms that the cis-activation capacity of the multifunctional molecule could be maintained with any anti-PD-1 binding domain.

### Example 7. Anti-PD-1/AntiCD28 Multifunctional Molecule can Reinvigorate Human Tumor-Infiltrating Lymphocytes

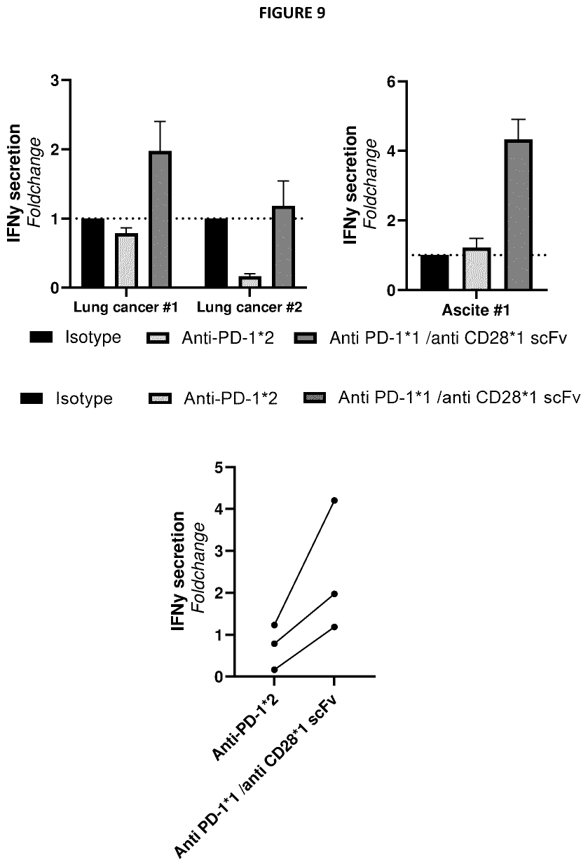

Biological activity of the anti-PD-1/anti-CD28 multifunctional molecule was next evaluated ex vivo on TILs isolated from fresh human tumor and ascites. TILs (Tumor-infiltrating lymphocytes) highly express PD-1 due to chronic tumor antigen stimulation occurring into the Tumor microenvironment. High expression of inhibitory receptor induces exhaustion of T cells that limit their effector functions and capacity to eliminate tumor cells. While anti-PD-1 therapeutic antibodies allow to reinvigorate T cells, their effect is generally not durable. The absence of co-stimulation is one limiting factor of the full reactivation of TILs. To evaluate whether the anti-PD-1/anti-CD28 multifunctional molecule can reinvigorate exhausted T cells, an ex vivo culture of fresh tumor/ascites in the presence of anti-PD-1/anti-CD28 multifunctional molecules was performed. Cells were cultivated in organoid system to maintain structural architecture of the tumor and microenvironment and IFNg secretion in the culture supernatant was quantified by MSD, 48 hours following treatment. As shown in FIG. 9, anti-PD-1/anti-CD28 multifunctional molecule (Format B1) enhance secretion of IFNg by TILs, confirming bioactivity of the multifunctional molecules to reinvigorate exhausted TILs.

### Example 8. Multiple Formats of Anti-PD-1/Anti-CD28 Multifunctional Molecules have a Good Pharmacokinetic in Mice

Clinical development of Bispecific drug is often limited by their bad pharmacokinetic profile associated with high clearance that can be governed by molecular weight, physicochemical properties, interaction with Fc receptors (Datta-Mannan et al., 2022, Chen et al., 2017). Pharmacokinetic of the present invention anti-PD-1/AntiCD28 antibodies in mice were investigated in C57BL/6JrJ wild-type mice that do not express human PD-1 nor human CD28 receptor. Mice received an intravenous single dose of anti-PD-1/anti-CD28 (4 mg/kg; 23 to 30 nM/kg) or anti-PD-1*2 (5 mg/kg; 35 nM/kg). Blood was sampled at different timing and antibodies concentrations were dosed by ELISA in sera using an anti-human Kappa and an anti-human Fc antibody for revelation.

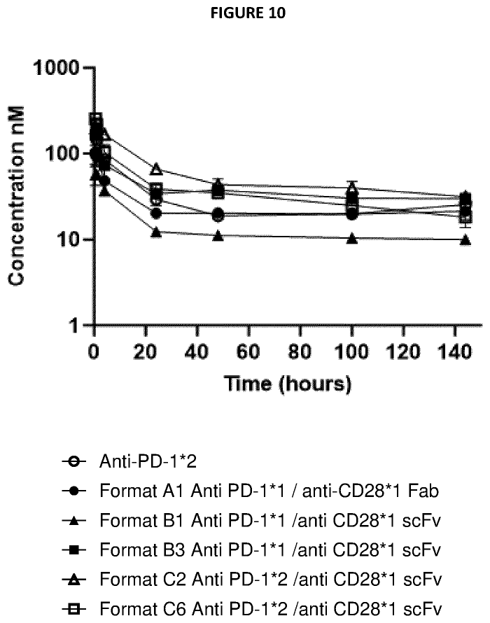

FIG. 10 shows that all formats of anti-PD-1/anti-CD28 antibodies present a similar pharmacokinetic than a conventional anti-PD-1 IgG4 antibody.

1-29. (canceled)

30. A multifunctional molecule comprising:
(a) a first binding moiety, said first binding moiety binding to a target specifically expressed on T cells surface selected from the group consisting of PD-1, VISTA, CTLA-4, BTLA, TIGIT, CD160, LAG3 and TIM3; and
(b) a second binding moiety, said second binding moiety having an agonistic effect on CD28 and being an anti-CD28 antigen binding domain comprising a heavy chain variable domain (VH) and a light chain variable domain (VL) and optionally a heavy chain constant domain (CH1) and a light chain constant domain (CL), wherein:
(i) the heavy chain variable domain (VH) comprises complementary determining regions (CDRs) HCDR1, HCDR2 and HCDR3, and
(ii) the light chain variable domain (VL) comprises complementary determining regions (CDRs) LCDR1, LCDR2 and LCDR3,
wherein:
the heavy chain CDR1 (HCDR1) comprises or consists of an amino acid sequence of SEQ ID NO: 30, optionally with one, two or three modification(s) selected from substitution(s), addition(s), deletion(s) and any combination thereof;
the heavy chain CDR2 (HCDR2) comprises or consists of an amino acid sequence of SEQ ID NO: 31, optionally with one, two or three modification(s) selected from substitution(s), addition(s), deletion(s) and any combination thereof;
the heavy chain CDR3 (HCDR3) comprises or consists of an amino acid sequence of SEQ ID NO: 32; optionally with one, two or three modification(s) selected from substitution(s), addition(s), deletion(s) and any combination thereof;
the light chain CDR1 (LCDR1) comprises or consists of an amino acid sequence of SEQ ID NO: 33, optionally with one, two or three modification(s) selected from substitution(s), addition(s), deletion(s) and any combination thereof;
the light chain CDR2 (LCDR2) comprises or consists of an amino acid sequence of SEQ ID NO: 34, optionally with one, two or three modification(s) selected from substitution(s), addition(s), deletion(s) and any combination thereof; and
the light chain CDR3 (LCDR3) comprises or consists of an amino acid sequence of SEQ ID NO: 35, optionally with one, two or three modification(s) selected from substitution(s), addition(s), deletion(s) and any combination thereof;
wherein said multifunctional molecule comprises a single anti-CD28 antigen binding domain.

31. The multifunctional molecule of claim 30, wherein the target specifically expressed on T cells surface is PD-1.

32. The multifunctional molecule of claim 30, wherein the first binding moiety is a first antigen binding domain comprising a heavy chain variable domain (VH) and a light chain variable domain (VL) and optionally a light chain constant domain (CL) and a first heavy chain constant domain (CH1), and wherein said first antigen binding domain is linked to the N-terminal end of a first Fc chain and the molecule further comprises a second Fc chain forming with the first Fc chain a Fc domain.

33. The multifunctional molecule of claim 30, wherein the anti-CD28 antigen binding domain is a Fab, a CrossMAb or a scFv.

34. The multifunctional molecule of claim 30, wherein the anti-CD28 antigen binding domain is a scFv and comprises:
from the N-terminal end to the C-terminal end, a VH linked to a VL, optionally by a peptide linker; or
from the N-terminal end to the C-terminal end, a VL linked to a VH, optionally by a peptide linker.

35. The multifunctional molecule of claim 30, wherein:
(i) the heavy chain variable domain (VH) further comprises heavy chain variable region framework regions (HFR) HFR1, HFR2, HFR3 and HFR4 comprising or consisting of an amino acid sequence of SEQ ID NOs: 36, 37, 38 and 39, respectively, optionally with one, two or three modification(s) selected from substitution(s), addition(s), deletion(s) and any combination thereof, and
(ii) the light chain variable domain (VL) further comprises region framework regions (LFR) LFR1, LFR2, LFR3 and LFR4, comprising or consisting of an amino acid sequence of SEQ ID NOs: 40, 41, 42 and 43, respectively, optionally with one, two or three modification(s) selected from substitution(s), addition(s), deletion(s) and any combination thereof.

36. The multifunctional molecule of claim 30, wherein the anti-CD28 antigen binding domain comprises: (a) a heavy chain variable region (VH) comprising or consisting of an amino acid sequence selected from the group consisting of SEQ ID NOs: 44, 83, 84, 85 and 88, optionally with one, two or three amino acid modification(s) selected from substitution(s), addition(s), deletion(s) and any combination thereof; and (b) a light chain variable region (VL) comprising or consisting of an amino acid sequence selected from the group consisting of SEQ ID NOs: 45, 86, 87, 89 and 90, optionally with one, two or three amino acid modification(s) selected from substitution(s), addition(s), deletion(s) and any combination thereof.

37. The multifunctional molecule of claim 30, wherein the anti-CD28 antigen binding domain comprises: (a) a VH comprising or consisting of an amino acid sequence of SEQ ID NO: 44, optionally with one, two or three modification(s) selected from substitution(s), addition(s), deletion(s) and any combination thereof in the framework regions; and (b) a VL comprising or consisting of an amino acid sequence of SEQ ID NO: 45, optionally with one, two or three modification(s) selected from substitution(s), addition(s), deletion(s) and any combination thereof in the framework regions.

38. The multifunctional molecule of claim 32, wherein the anti-CD28 antigen binding domain is covalently linked to: (i) the C-terminal end of a CL of the first binding moiety, (ii) the C-terminal end of a CH1 of the first binding moiety, (iii) the N-terminal end of a VH of the first binding moiety, or (iii) the N-terminal end of a VL of the first binding moiety, optionally through a peptide linker.

39. The multifunctional molecule of claim 32, wherein the molecule comprises a first Fc chain and a second Fc chain forming together a Fc domain and the anti-CD28 antigen binding domain is covalently linked to the C-terminal end of one of the first Fc chain which is covalently linked at its N-terminal end to the first antigen binding domain, optionally through a peptide linker.

40. The multifunctional molecule of claim 30, wherein the molecule comprises a first Fc chain and a second Fc chain forming together a Fc domain, the anti-CD28 antigen binding domain is covalently linked by its C-terminal end to N-terminal end of the second Fc chain and the first antigen binding domain is covalently linked by its C-terminal end to N-terminal end of the first Fc chain, optionally through a peptide linker.

41. The multifunctional molecule of claim 30, wherein the first binding moiety is or is from an anti-PD-1 antibody selected from the group consisting of Pembrolizumab, Nivolumab, Pidilizumab, Cemiplimab, Camrelizumab, AUNP12, AMP-224, AGEN-2034, Tisleizumab, PDR001, MK-3477, PF-06801591, JNJ-63723283, genolimzumab, LZM-009, BCD-100, SHR-1201, BAT-1306, AK-103, MEDI-0680, JS001, BI-754091, CBT-501, INCSHR1210, TSR-042, GLS-010, AM-0001, STI-1110, MGA012, and IBI308.

42. The multifunctional molecule of claim 30, wherein the first binding moiety is an anti-PD-1 antigen binding domain comprising:
(i) a VH comprising HCDR1, HCDR2 and HCDR3, and (ii) a VL comprising LCDR1, LCDR2 and LCDR3,
wherein:
the heavy chain CDR1 (HCDR1) comprises or consists of an amino acid sequence of SEQ ID NO: 1;
the heavy chain CDR2 (HCDR2) comprises or consists of an amino acid sequence of SEQ ID NO: 2;
the heavy chain CDR3 (HCDR3) comprises or consists of an amino acid sequence of SEQ ID NO: 3;
the light chain CDR1 (LCDR1) comprises or consists of an amino acid sequence of SEQ ID NO: 4;
the light chain CDR2 (LCDR2) comprises or consists of an amino acid sequence of SEQ ID NO: 5, and
the light chain CDR3 (LCDR3) comprises or consists of an amino acid sequence of SEQ ID NO: 6.

43. The multifunctional molecule of claim 30, wherein the first binding moiety is an anti-PD-1 antigen binding domain comprising: (a) a VH comprising or consisting of an amino acid sequence of SEQ ID NO: 15, optionally with one, two or three modification(s) selected from substitution(s), addition(s), deletion(s) and any combination thereof in the framework regions; and (b) a VL comprising or consisting of an amino acid sequence of SEQ ID NO: 16, optionally with one, two or three modification(s) selected from substitution(s), addition(s), deletion(s) and any combination thereof in the framework regions.

44. The multifunctional molecule of claim 30, wherein the first binding moiety is an anti-PD-1 antigen binding domain comprising:
a) (i) a VH comprising HCDR1, HCDR2 and HCDR3, and (ii) a VL comprising LCDR1, LCDR2 and LCDR3, wherein: the heavy chain CDR1 (HCDR1) comprises or consists of an amino acid sequence of SEQ ID NO: 65; the heavy chain CDR2 (HCDR2) comprises or consists of an amino acid sequence of SEQ ID NO: 66; the heavy chain CDR3 (HCDR3) comprises or consists of an amino acid sequence of SEQ ID NO: 67; the light chain CDR1 (LCDR1) comprises or consists of an amino acid sequence of SEQ ID NO: 68; the light chain CDR2 (LCDR2) comprises or consists of an amino acid sequence of SEQ ID NO: 69, and the light chain CDR3 (LCDR3) comprises or consists of an amino acid sequence of SEQ ID NO: 70; or
b) i) a VH comprising or consisting of an amino acid sequence of SEQ ID NO: 71; and ii) a VL comprising or consisting of an amino acid sequence of SEQ ID NO: 72; or
c) (i) a VH comprising HCDR1, HCDR2 and HCDR3, and (ii) a VL comprising LCDR1, LCDR2 and LCDR3, wherein: the heavy chain CDR1 (HCDR1) comprises or consists of an amino acid sequence of SEQ ID NO: 73; the heavy chain CDR2 (HCDR2) comprises or consists of an amino acid sequence of SEQ ID NO: 74; the heavy chain CDR3 (HCDR3) comprises or consists of an amino acid sequence of SEQ ID NO: 75; the light chain CDR1 (LCDR1) comprises or consists of an amino acid sequence of SEQ ID NO: 76; the light chain CDR2 (LCDR2) comprises or consists of an amino acid sequence of SEQ ID NO: 77, and the light chain CDR3 (LCDR3) comprises or consists of an amino acid sequence of SEQ ID NO: 78; or
d) i) a VH comprising or consisting of an amino acid sequence of SEQ ID NO: 79; and ii) a VL comprising or consisting of an amino acid sequence of SEQ ID NO: 80.

45. The multifunctional molecule of claim 30, wherein the anti-PD-1 antigen binding domain is a F(ab′)2, a Fab or a CrossMAb.

46. An isolated nucleic acid sequence or a group of isolated nucleic acid sequences encoding the multifunctional molecule of claim 30 or a vector comprising said nucleic acid sequence or group of isolated nucleic acid sequences.

47. A host cell, comprising the isolated nucleic acid, group of nucleic acid molecules, or vector of claim 46.

48. A method for producing the multifunctional molecule comprising a step of culturing a host cell of claim 47 and a step of isolating the multifunctional molecule.

49. A pharmaceutical composition comprising the multifunctional molecule of claim 30 and a pharmaceutically acceptable carrier.

50. A method for treating a cancer or an infectious disease in a subject in need thereof, comprising the administration of a multifunctional molecule of claim 30 or a pharmaceutical composition comprising said multifunctional molecule and a pharmaceutically acceptable carrier.
